(19) United States
(12) Patent Application Publication
(10) Pub. No.: US 2010/0124756 A1
(43) Pub. Date: May 20, 2010

(54) COLLECTION OF BIOMARKERS FOR DIAGNOSIS AND MONITORING OF ALZHEIMER'S DISEASE IN BODY FLUIDS

(76) Inventors: Sandip Ray, San Francisco, CA (US); Anton Wyss-Coray, Stanford, CA (US)

Correspondence Address: MORRISON & FOERSTER LLP, 755 PAGE MILL RD, PALO ALTO, CA, 94304-1018, US

(21) Appl. No.: 12/577,082

(22) Filed: Oct. 9, 2009

Related U.S. Application Data
(60) Provisional application No. 61/195,776, filed on Oct. 10, 2008.

(51) Int. Cl. G01N 33/53; C12M 1/34

(52) U.S. Cl. 435/7.94; 435/287.9

(57) ABSTRACT

The inventors have discovered sets of proteinaceous biomarkers (“AD biomarkers”) which can be measured in peripheral biological fluid samples to aid in the diagnosis of neurodegenerative disorders, particularly Alzheimer's disease. The invention further provides methods of identifying candidate agents for the treatment of Alzheimer's disease by testing prospective agents for activity in modulating the levels of the AD biomarkers.

### CROSS-REFERENCE TO RELATED APPLICATIONS

This application claims the benefit under 35 U.S.C. §119(e) of U.S. Provisional Application Ser. No. 61/195,776, filed Oct. 10, 2008, the contents of which are incorporated herein by reference in their entirety.

### STATEMENT REGARDING FEDERALLY SPONSORED RESEARCH

This study was supported by the John Douglas French Alzheimer's Foundation, the NIH (AG20603), an Alzheimer Center Grant (NIA-AG08017), and the Veterans Administration Geriatric Research, Education and Clinical Center. We also acknowledge the support from the Veterans Administration Mental Illness Research, Education and Clinical Center and the various Alzheimer's Centers sponsored by the National Institute of Aging. The Federal Government may have certain rights in this invention.

### BACKGROUND OF THE INVENTION

An estimated 4.5 million Americans have Alzheimer's Disease (“AD”). By 2050, the estimated range of AD prevalence will be 11.3 million to 16 million. Currently, the societal cost of AD to the U.S. is $100 billion per year, including $61 billion borne by U.S. businesses. Neither Medicare nor most private health insurance covers the long-term care most patients need.

Alzheimer's Disease is a neurodegenerative disease of the central nervous system associated with progressive memory loss resulting in dementia. Two pathological characteristics are observed in AD patients at autopsy: extracellular plaques and intracellular tangles in the hippocampus, cerebral cortex, and other areas of the brain essential for cognitive function. Plaques are formed mostly from the deposition of amyloid beta (“Aβ”), a peptide derived from amyloid precursor protein (“APP”). Filamentous tangles are formed from paired helical filaments composed of neurofilament and hyperphosphorylated tau protein, a microtubule-associated protein. It is not clear, however, whether these two pathological changes are only associated with the disease or truly involved in the degenerative process. Late-onset/sporadic AD has virtually identical pathology to inherited early-onset/familial AD (FAD), thus suggesting common pathogenic pathways for both forms of AD. To date, genetic studies have identified three genes that cause autosomal dominant, early-onset AD, amyloid precursor protein (“APP”), presenilin 1 (“PS1”), and presenilin 2 (“PS2”). A fourth gene, apolipoprotein E (“ApoE”), is the strongest and most common genetic risk factor for AD, but does not necessarily cause it. All mutations associated with APP and PS proteins can lead to an increase in the production of Aβ peptides, specifically the more amyloidogenic form, Aβ₄₂. In addition to genetic influences on amyloid plaque and intracellular tangle formation, environmental factors (e.g., cytokines, neurotoxins, etc.) may also play important roles in the development and progression of AD.

The main clinical feature of AD is a progressive cognitive decline leading to memory loss. The memory dysfunction involves impairment of learning new information which is often characterized as short-term memory loss. In the early (mild) and moderate stages of the illness, recall of remote well-learned material may appear to be preserved, but new information cannot be adequately incorporated into memory. Disorientation to time is closely related to memory disturbance.

Language impairments are also a prominent part of AD. These are often manifest first as word finding difficulty in spontaneous speech. The language of the AD patient is often vague, lacking in specifics and may have increased automatic phrases and clichés. Difficulty in naming everyday objects is often prominent. Complex deficits in visual function are present in many AD patients, as are other focal cognitive deficits such as apraxia, acalculia and left-right disorientation. Impairments of judgment and problems solving are frequently seen.

Non-cognitive or behavioral symptoms are also common in AD and may account for an event larger proportion of caregiver burden or stress than the cognitive dysfunction. Personality changes are commonly reported and range from progressive passivity to marked agitation. Patients may exhibit changes such as decreased expressions of affection. Depressive symptoms are present in up to 40%. A similar rate for anxiety has also been recognized. Psychosis occurs in 25%. In some cases, personality changes may predate cognitive abnormality.

Currently, the primary method of diagnosing AD in living patients involves taking detailed patient histories, administering memory and psychological tests, and ruling out other explanations for memory loss, including temporary (e.g., depression or vitamin B₁₂ deficiency) or permanent (e.g., stroke) conditions. These clinical diagnostic methods, however, are not foolproof.

One obstacle to diagnosis is pinpointing the type of dementia; AD is only one of seventy conditions that produce dementia. Because of this, AD cannot be diagnosed with complete accuracy until after death, when autopsy reveals the disease's characteristic amyloid plaques and neurofibrillary tangles in a patient's brain. In addition, clinical diagnostic procedures are only helpful after patients have begun displaying significant, abnormal memory loss or personality changes. By then, a patient has likely had AD for years.

Given the magnitude of the public health problem posed by AD, considerable research efforts have been undertaken to elucidate the etiology of AD as well as to identify biomarkers (secreted proteins or metabolites) that can be used to diagnose and/or predict whether a person is likely to develop AD. Because AD the CNS is relatively isolated from the other organs and systems of the body, most research (in regards to both disease etiology and biomarkers) has focused on events, gene expression, biomarkers, etc. within the central nervous system. With regards to biomarkers, the proteins amyloid beta and tau are probably the most well characterized. Research has shown that cerebrospinal fluid (“CSF”) samples from AD patients contain higher than normal amounts of tau, which is released as neurons degenerate, and lower than normal amounts of beta amyloid, presumably because it is trapped in the brain in the form of amyloid plaques. Because these biomarkers are released into CSF, a lumbar puncture (or “spinal tap”) is required to obtain a sample for testing.

A number of U.S. patents and applications have been published relating to methods for diagnosing AD, including U.S. Pat. Nos. 4,728,605, 5,874,312, 6,027,896, 6,114,133, 6,130,048, 6, 210, 895, 6,358,681, 6,451,547, 6,461,831, 6,465,195, 6,475,161, and 6,495,335, and U.S. patent application Ser. Nos. 11/580,405, 11/148,595, and 10/993,813. Additionally, a number of reports in the scientific literature relate to certain biochemical markers and their correlation/association with AD, including Fahnestock et al., 2002, J. Neural. Transm. Suppl. 2002(62):241-52; Masliah et al., 1195, Neurobiol. Aging 16(4):549-56; Power et al., 2001, Dement. Geriatr. Cogn. Disord. 12(2):167-70; and Burbach et al., 2004, J. Neurosci. 24(10):2421-30. Additionally, Li et al. (2002, Neuroscience 113(3):607-15) and Sanna et al. (2003, J. Clin. Invest. 111(2):241-50) have investigated Leptin in relation to memory and multiple sclerosis, respectively.

All patents and publications cited herein are incorporated by reference in their entirety.

### BRIEF SUMMARY OF THE INVENTION

The inventors have discovered sets or groups of biochemical markers, present in the blood of individuals, including from the serum or plasma of individuals, which are altered in individuals with Alzheimer's Disease (“AD”). Accordingly, these sets of biomarkers (“AD diagnosis biomarkers”) may be used to diagnose or aid in the diagnosis of AD and/or to measure progression of AD in AD patients. The invention provides methods for the diagnosis of AD or aiding the diagnosis of AD in an individual by measuring the amount of each AD diagnosis biomarker in the set in a biological fluid sample, such as a peripheral biological fluid sample from the individual and comparing the measured amount with a reference value for each AD diagnosis biomarker measured. The information thus obtained may be used to aid in the diagnosis or to diagnose AD in the individual.

Various embodiments are described with reference to certain aspects of the invention. However, it is to be understood that the various embodiments described herein may be used in other aspects of the invention as described herein, as will be apparent to one of ordinary skill in the art.

Accordingly, the present invention provides a method aiding diagnosis of Alzheimer's disease (“AD”), comprising comparing a measured level of at least sixteen AD diagnosis biomarkers in a biological fluid sample from an individual seeking a diagnosis for AD to a reference level for each biomarker, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha). In some embodiments, the method comprises comparing the measured value to a reference value for each AD diagnosis biomarker measured comprises calculating the fold difference between the measured value and the reference value. In some embodiments, the method comprises comparing the fold difference for each AD diagnosis biomarker measured with a minimum fold difference value. In some embodiments, said biological fluid sample is a peripheral biological fluid sample. In some embodiments, said peripheral biological fluid sample is blood, serum or plasma. In some embodiments, said peripheral biological fluid sample is serum or plasma. In some embodiments, the method comprises obtaining a measured level of said AD biomarker in said biological fluid sample. In some embodiments, the individual is a human. In some embodiments, the measured levels are normalized. In some embodiments, the reference levels are obtained from measured values of the at least sixteen biomarkers from samples in the blood of human individuals without AD. In some embodiments, the reference levels are obtained from measured values of the at least sixteen biomarkers from samples in the blood of human individuals with AD. In some embodiments, the reference levels are normalized. In some embodiments, the method comprises comparing the measured level of the at least sixteen AD diagnosis biomarkers to two reference levels for each biomarker. In some embodiments, the two reference levels for each biomarker comprise: (a) a reference level obtained from measured values of the at least sixteen biomarkers from samples in the blood of human individuals without AD; and (b) a reference level obtained from measured values of the at least sixteen biomarkers from samples in the blood of human individuals with AD. In some embodiments, the group of individuals without AD is a control population selected from an age-matched population, a degenerative control population, a non-AD neurodegenerative control population, a healthy age-matched control population, or a mixed population thereof. In some embodiments, comparing comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier. In some embodiments, comparing comprises a method comprising predication analysis of microarray (PAM). In some embodiments, the method comprises formulating a decision tree; and using the decision tree for classification of the sample from the individual seeking AD diagnosis, wherein the classification aids the diagnosis of AD. In some embodiments, using the decision tree for classification of the sample is implemented by a computer. In some embodiments, the diagnosis of AD is aided by determining a difference between the measured levels of the at least sixteen AD diagnosis biomarkers to the reference levels of the at least sixteen biomarkers wherein the difference meets or exceeds a statistically significant difference between normalized measured values of the at least sixteen AD diagnosis biomarkers in the blood samples from individuals without AD and individuals with AD, wherein the statistically significant difference indicates a diagnosis of AD, wherein the measured levels are normalized, and wherein the references levels are normalized. In some embodiments, the method comprises: formulating a decision tree comprising statistically significant differences in normalized measured values of AD diagnosis biomarkers wherein the statistically significant differences are determined from normalized measured values of the plurality of AD diagnosis biomarkers in the blood samples in the group of individuals with AD and the group of individuals without AD; and using the decision tree for classification of the blood sample from the individual seeking AD diagnosis, wherein the classification aids the diagnosis of AD. In some embodiments, the method is useful for early detection of conversion of MCI to AD. In some embodiments, the at least sixteen AD diagnosis biomarkers further comprise: TRAIL R4, and IGFBP-6. In some embodiments, the method comprises the step of obtaining a value for each comparison of the measured level to the reference level.

In some embodiments, the method for aiding diagnosis of Alzheimer's disease (“AD”), comprises: comparing normalized measured levels of at least sixteen AD diagnosis biomarkers in a blood sample from a human individual seeking a diagnosis for AD to reference levels for the at least sixteen biomarkers in the blood sample, wherein the reference levels are obtained from normalized measured values of the at least sixteen biomarkers from samples in the blood of human individuals without AD, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha), whereby the diagnosis of AD is aided by determining a difference between the normalized measured levels of the at least sixteen AD diagnosis biomarkers to the reference levels of the at least sixteen biomarkers from non-AD samples wherein the difference meets or exceeds a statistically significant difference between normalized measured values of the at least sixteen AD diagnosis biomarkers in the blood samples from individuals without AD and individuals with AD, wherein the statistically significant difference indicates a diagnosis of AD. In some embodiments, the blood sample is serum or plasma. In some embodiments, the reference levels for the at least sixteen biomarkers are obtained by a method comprising: determining normalized measured levels of the at least sixteen biomarkers in normal individuals with a Mini Mental State Examination (MMSE) score greater than 25, having a statistically significant difference from normalized measured levels of the at least sixteen biomarkers in AD subjects with MMSE score of 25 and below. In some embodiments, the reference levels for the at least sixteen biomarkers are obtained by a method comprising: determining normalized measured levels of the at least sixteen biomarkers in normal individuals, having a statistically significant difference from normalized measured levels of the at least sixteen biomarkers in AD individuals, wherein the individuals are classified as normal or AD by clinical diagnosis. In some embodiments, the statistically significant difference in normalized measured levels of the at least sixteen AD diagnosis biomarkers in samples from individuals with AD relative to samples from individuals without AD is determined by a method comprising Significance Analysis of Microarrays (SAM). In some embodiments, the statistically significant difference in normalized measured levels of the at least sixteen AD diagnosis biomarkers determined by SAM has a q-value range from about 0 to about 0.05. In some embodiments, the statistically significant difference in normalized measured levels of the at least sixteen AD diagnosis biomarkers in samples from individuals with AD relative to samples from individuals without AD is determined by a method comprising at test. In some embodiments, the statistically significant difference is measured in terms of a p-value. In some embodiments, comparing the measured values comprises a method selected from the group consisting of Significance Analysis of Microarrays, Tree Harvesting, CART, MARS, Self Organizing Maps, Frequent Item Set, Bayesian networks, Prediction Analysis of Microarray (PAM), SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier. In some embodiments, the aiding the diagnosis of AD further comprises one or more clinical diagnostic methods comprising taking patient histories, administering memory tests, attributing a MMSE score, administering psychological tests, or ruling out temporary or permanent conditions that may explain memory loss. In some embodiments, determining the statistically significant difference associated with a diagnosis of AD comprises: determining a mean, median, or shrunken centroid value of normalized measured values of the at least sixteen AD diagnosis biomarkers in the blood samples from a group of individuals with AD; determining a mean, median, or shrunken centroid value of normalized measured values of the at least sixteen AD diagnosis biomarkers in the blood samples from a group of individuals without AD; and finding a statistically significant difference between the mean, median, or shrunken centroid values of the normalized measured values of the at least sixteen AD diagnosis biomarkers in the blood samples between the two groups. In some embodiments, parameters for the statistically significant difference comprise one or more of: a correlation of greater than 90% (r=0.9 to r=0.99); a p-value between 0 and 0.05; a fold change in levels of greater than 20%; and a score (d) of greater than 1 for markers whose levels increase and less than 1 for markers whose levels decrease. In some embodiments, the group of individuals without AD is a control population selected from an age-matched population, a degenerative control population, a non-AD neurodegenerative control population, a healthy age-matched control population, or a mixed population thereof. In some embodiments, the method comprises formulating a decision tree comprising statistically significant differences in normalized measured values of AD diagnosis biomarkers wherein the statistically significant differences are determined from normalized measured values of the plurality of AD diagnosis biomarkers in the blood samples in the group of individuals with AD and the group of individuals without AD; and using the decision tree for classification of the blood sample from the individual seeking AD diagnosis, wherein the classification aids the diagnosis of AD. In some embodiments, the normalized measured values of the plurality of AD diagnosis biomarkers in the blood samples from the group of individuals with AD and the group of individuals without AD form statistically significant differences in normalized measured values for learning samples. In some embodiments, the method comprises comparing the statistically significant differences in normalized measured levels of AD diagnosis biomarkers in the blood sample from the individual seeking AD diagnosis, with the statistically significant differences in normalized measured values for learning samples. In some embodiments, using the decision tree for classification of the blood sample is implemented by a computer. In some embodiments, determining the reference level further comprises: selecting biomarkers with p-values less than or equal to 5%; and using cluster analysis to classify samples from individuals with AD. In some embodiments, the at least sixteen AD diagnosis biomarkers further comprise: TRAIL R4 (TNF-related apoptosis-inducing ligand receptor 4) and IGFBP-6 (insulin-like growth factor binding protein 6).

In another aspect of the invention is a method for monitoring progression of Alzheimer's disease (AD) in an AD patient, comprising: comparing a measured level of at least sixteen AD diagnosis biomarkers in a biological fluid sample from an individual seeking a diagnosis for AD to a reference level for each biomarker, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11(interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha). In some embodiments, the method comprises comparing the measured value to a reference value for each AD diagnosis biomarker measured comprises calculating the fold difference between the measured value and the reference value. In some embodiments, the method comprises comparing the fold difference for each AD diagnosis biomarker measured with a minimum fold difference value. In some embodiments, said biological fluid sample is a peripheral biological fluid sample. In some embodiments, said peripheral biological fluid sample is blood, serum or plasma. In some embodiments, said peripheral biological fluid sample is serum or plasma. In some embodiments, the method comprises obtaining a measured level of said AD biomarker in said biological fluid sample. In some embodiments, the individual is a human. In some embodiments, the measured levels are normalized. In some embodiments, said reference level is a level obtained from a biological fluid sample from the same AD patient at an earlier point in time. In some embodiments, the reference levels are obtained from measured values of the at least sixteen biomarkers from samples in the blood of human individuals without AD. In some embodiments, the reference levels are obtained from measured values of the at least sixteen biomarkers from samples in the blood of human individuals with AD. In some embodiments, the reference levels are normalized. In some embodiments, the method comprises comparing the measured level of the at least sixteen AD diagnosis biomarkers to two reference levels for each biomarker. In some embodiments, the two reference levels for each biomarker comprise: (a) a reference level obtained from measured values of the at least sixteen biomarkers from samples in the blood of human individuals without AD; and (b) a reference level obtained from measured values of the at least sixteen biomarkers from samples in the blood of human individuals with AD. In some embodiments, the group of individuals without AD is a control population selected from an age-matched population, a degenerative control population, a non-AD neurodegenerative control population, a healthy age-matched control population, or a mixed population thereof. In some embodiments, comparing comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier. In some embodiments, comparing comprises a method comprising predication analysis of microarray (PAM). In some embodiments, the method comprises formulating a decision tree; and using the decision tree for classification of the sample from the individual seeking AD diagnosis, wherein the classification aids the diagnosis of AD. In some embodiments, using the decision tree for classification of the sample is implemented by a computer. In some embodiments, the diagnosis of AD is aided by determining a difference between the measured levels of the at least sixteen AD diagnosis biomarkers to the reference levels of the at least sixteen biomarkers wherein the difference meets or exceeds a statistically significant difference between normalized measured values of the at least sixteen AD diagnosis biomarkers in the blood samples from individuals without AD and individuals with AD, wherein the statistically significant difference indicates a diagnosis of AD, wherein the measured levels are normalized, and wherein the references levels are normalized. In some embodiments, the method comprises formulating a decision tree comprising statistically significant differences in normalized measured values of AD diagnosis biomarkers wherein the statistically significant differences are determined from normalized measured values of the plurality of AD diagnosis biomarkers in the blood samples in the group of individuals with AD and the group of individuals without AD; and using the decision tree for classification of the blood sample from the individual seeking AD diagnosis, wherein the classification aids the diagnosis of AD. In some embodiments, the method is useful for early detection of conversion of MCI to AD. In some embodiments, the at least sixteen AD diagnosis biomarkers further comprise: TRAIL R4, and IGFBP-6. In some embodiments, the method comprises the step of obtaining a value for each comparison of the measured level to the reference level.

In some embodiments, the method for monitoring progression of Alzheimer's disease (AD) in an AD patient, comprises comparing normalized measured levels of at least sixteen AD diagnosis biomarkers in a blood sample from a human individual with AD to reference levels for the at least sixteen biomarkers in the blood sample, wherein the reference levels are obtained from normalized measured values of the at least sixteen biomarkers from samples in the blood of human individuals without AD, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha), whereby the progression of AD is monitored by determining a difference between the normalized measured levels of the at least sixteen biomarkers to the reference levels of the at least sixteen biomarkers from non-AD samples wherein the difference meets or exceeds a statistically significant difference between normalized measured values of the at least sixteen biomarkers in the blood samples from individuals without AD and individuals with AD, wherein the statistically significant difference indicates a progression of AD. In some embodiments, the blood sample is serum or plasma. In some embodiments, the individual with AD is an individual with questionable AD and scored or would achieve a score of 25-28 upon MMSE testing. In some embodiments, the individual with AD is an individual with mild AD and scored or would achieve a score of 22-27 upon MMSE testing. In some embodiments, the individual with AD is an individual with moderate AD and scored or would achieve a score of 16-21 upon MMSE testing. In some embodiments, the individual with AD is an individual with severe AD and scored or would achieve a score of less than 12-15 upon MMSE testing. In some embodiments, the reference levels for the at least sixteen biomarkers are obtained by a method comprising: determining normalized measured levels of the at least sixteen biomarkers in normal individuals with a Mini Mental State Examination (MMSE) score greater than 25, having a statistically significant difference from normalized measured levels of the at least sixteen biomarkers in AD subjects with MMSE score of 25 and below. In some embodiments, the reference levels for the at least sixteen biomarkers are obtained by a method comprising: determining normalized measured levels of the at least sixteen biomarkers in normal individuals, having a statistically significant difference from normalized measured levels of the at least sixteen biomarkers in AD individuals, wherein the individuals are classified as normal or AD by clinical diagnosis. In some embodiments, the statistically significant difference in normalized measured levels of the at least sixteen AD diagnosis biomarkers in samples from individuals with AD relative to samples from individuals without AD is determined by a method comprising Significance Analysis of Microarrays (SAM). In some embodiments, the statistically significant difference in normalized measured levels of the at least sixteen AD diagnosis biomarkers determined by SAM comprises a q-value range from about 0 to about 0.05. In some embodiments, the statistically significant difference in normalized measured levels of the at least sixteen AD diagnosis biomarkers in samples from individuals with AD relative to samples from individuals without AD is determined by a method comprising at test. In some embodiments, the statistically significant difference is measured in terms of a p-value. In some embodiments, the p-value is between about 0 to about 0.05. In some embodiments, the normalized measured level is normalized relative to a median value determined contemporaneously using a pool of samples from individuals with AD and individuals without AD which includes the sample from the individual. In some embodiments, comparing the measured values comprises a method selected from the group consisting of Significance Analysis of Microarrays, Tree Harvesting, CART, MARS, Self Organizing Maps, Frequent Item Set, Bayesian networks, Prediction Analysis of Microarray (PAM), SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier. In some embodiments, determining the statistically significant difference associated with monitoring progression of AD comprises: determining a mean, median, or shrunken centroid value of normalized measured values of the at least sixteen AD diagnosis biomarkers in the blood samples from a group of individuals with AD; determining a mean, median, or shrunken centroid value of normalized measured values of the at least sixteen AD diagnosis biomarkers in the blood samples from a group of individuals without AD; and finding a statistically significant difference between the mean, median, or shrunken centroid values of the normalized measured values of the at least sixteen AD diagnosis biomarkers in the blood samples between the two groups. In some embodiments, parameters for the statistically significant difference comprise one or more of: a correlation of greater than 90% (r=0.9 to r=0.99); a p-value between 0 and 0.05; a fold change in levels of greater than 20%; and a score (d) of greater than 1 for markers whose levels increase and less than 1 for markers whose levels decrease. In some embodiments, the group of individuals without AD is a control population selected from an age-matched population, a degenerative control population, a non-AD neurodegenerative control population, a healthy age-matched control population, or a mixed population thereof. In some embodiments, the method comprises: formulating a decision tree comprising statistically significant differences in normalized measured values of AD diagnosis biomarkers selected from the group listed in claim A19 wherein the statistically significant differences are determined from normalized measured values of the plurality of AD diagnosis biomarkers in the blood samples in the group of individuals with AD and the group of individuals without AD; and using the decision tree for classification of the blood sample from the individual seeking AD diagnosis, wherein the classification monitors the progression of AD. In some embodiments, the normalized measured values of the plurality of AD diagnosis biomarkers selected from the group listed in claim A19 in the blood samples from the group of individuals with AD and the group of individuals without AD form statistically significant differences in normalized measured values for learning samples. In some embodiments, the method comprises comparing the statistically significant differences in normalized measured levels of AD diagnosis biomarkers in the blood sample from the individual seeking AD diagnosis, with the statistically significant differences in normalized measured values for learning samples. In some embodiments, using the decision tree for classification of the blood sample is implemented by a computer. In some embodiments, determining the reference level further comprises: selecting biomarkers with p-values less than or equal to 5%; and using cluster analysis to classify samples from individuals with AD. In some embodiments, the at least sixteen AD diagnosis biomarkers further comprise: TRAIL R4 (TNF-related apoptosis-inducing ligand receptor 4) and IGFBP-6 (insulin-like growth factor binding protein 6).

In another aspect of the invention is a method of identifying a candidate agent for treatment of Alzheimer's Disease, comprising: assaying a prospective candidate agent for activity in modulating at least sixteen AD biomarkers, said AD biomarkers comprising: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha). In some embodiments, the at least sixteen AD diagnosis biomarkers further comprise: TRAIL R4, and IGFBP-6. In some embodiments, said assaying is performed in vivo.

The invention is also useful for detecting conversion from mild cognitive deficit (MCI) to AD, as well as predicting conversion from MCI to AD. MCI is a clinically recognized disorder considered distinct from AD in which cognition and memory are mildly deficient. Accordingly, the invention further provides a method for predicting or detecting conversion from MCI to AD, comprising: comparing a measured level of at least sixteen AD diagnosis biomarkers in a biological fluid sample from an individual seeking a diagnosis for AD to a reference level for each biomarker, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha). In some embodiments, the at least sixteen AD biomarkers further comprise TRAIL R4 and IGFBP-6. In some embodiments, the method is used to predict conversion from MCI to AD. In some embodiments, the method is used to detect conversion from MCI to AD.

In another aspect of the invention is a kit comprising: at least one reagent specific for each of at least sixteen AD diagnosis biomarkers, said at least sixteen AD diagnosis biomarkers comprising: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha; and instructions for carrying out a method as described herein. In some embodiments, the kit comprises at least one reagent specific for each of TRAIL R4 and IGFBP-6. In some embodiments, the reagents specific for the AD diagnosis biomarkers are antibodies, or fragments thereof, that are specific for said AD diagnosis biomarkers. In some embodiments, the kit comprises at least one reagent specific for a biomarker that measures sample characteristics. In some embodiments, the reagents are useful for a sandwich antibody array assay. The kits may be for use in any of the methods described herein, for example, aiding diagnosis of AD, monitoring progression of Alzheimer's disease (AD), and identifying candidate agents for treatment of Alzheimer's Disease. In some embodiments, the kits include at least one reagent specific for each AD diagnosis marker, where the AD diagnosis biomarkers comprise: MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6, and instructions for carrying out the method as described herein. Additionally, provided herein are sets of reference values for a set of AD diagnosis biomarkers comprising: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a, and a set of reagents specific for the set of AD diagnosis biomarkers comprising MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. Additionally, provided herein are sets of reference values for a set of AD diagnosis biomarkers comprising: MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6, and a set of reagents specific for the set of AD diagnosis biomarkers comprising MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6. In further examples of kits for use in the methods as disclosed herein, the reagents specific for the AD diagnosis biomarkers are antibodies, or fragments thereof, that are specific for said AD diagnosis biomarkers. In further examples, kits for use in the methods disclosed herein further comprise at least one reagent specific for a biomarker that measures sample characteristics. In further examples, the kit detects common variants of the biomarkers, wherein a common variant indicates a protein that is expressed in at least 5 percent or more of the population in industrialized nations. In further examples, a kit for use in the methods disclosed herein further comprises a biomarker for normalizing data. In some examples, the biomarker for normalizing data is selected from the group consisting of TGF-beta and TGF-beta3.

In another aspect of the invention is a surface comprising attached thereto, at least one reagent specific for each of at least sixteen AD diagnosis biomarkers, said at least sixteen AD diagnosis biomarkers comprising: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha. In some embodiments, the surface comprises at least one reagent specific for each of TRAIL R4 and IGFBP-6. In some embodiments, the kit comprises: at least one reagent specific for a biomarker that measures sample characteristics. In some embodiments, said reagents specific for said AD diagnosis biomarkers are antibodies, or fragments thereof, that are specific for said AD diagnosis biomarkers. In some embodiments, the surface is useful in a sandwich antibody array assay. Provided herein are surfaces comprising attached thereto, at least one reagent specific for each AD diagnosis biomarker in a set of AD diagnosis biomarkers, wherein said set of AD diagnosis biomarkers comprises MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a or the set of AD biomarkers comprises MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6; and at least one reagent specific for a biomarker that measures sample characteristics. In further examples, provided herein are surfaces wherein said reagents specific for said AD diagnosis biomarkers are antibodies, or fragments thereof, that are specific for said AD diagnosis biomarkers. The surfaces may be used in any of the methods described herein.

In another aspect of the invention is a combination comprising a surface as described herein and a peripheral biological fluid sample from an individual. In some embodiments, said individual is at least 60, 65, 70, 75, 80, or 85 years of age.

In another aspect of the invention is a computer readable format comprising the values and/or reference levels as obtained by a method described herein. Provided herein are methods for obtaining values for the comparison of the measured level to the reference level of biological fluid samples.

In any of the above methods, in some embodiments, the comparison of the measured value and the reference value includes calculating a fold difference between the measured value and the reference value. In some embodiments the measured value is obtained by measuring the level of the AD diagnosis biomarker(s) in the sample, while in other embodiments the measured value is obtained from a third party. In some embodiments, the peripheral biological fluid sample is a blood sample. In certain embodiments, the peripheral biological fluid sample is a plasma sample. In other embodiments, the peripheral biological fluid sample is a serum sample.

It is understood that aspect and embodiments of the invention described herein include “comprising”, “consisting of” and/or “consisting essentially of” aspects and embodiments.

### BRIEF DESCRIPTION OF THE DRAWINGS

FIG. 1 shows an example of a study outline for generating diagnostic test statistics.

FIG. 2 shows a “heat map” generated with an unsupervised cluster algorithm for 19 proteins with highly significant differences in expression (q-value <3.4%) between AD and NDC groups. Samples are arranged in columns, proteins in rows.

FIG. 3 shows predictor discovery by PAM, performed with normalized array measurements of 120 signaling proteins in the training set.

FIGS. 4A-4C show classification and prediction of clinical Alzheimer's diagnosis in subjects with Alzheimer's disease or MCI and functional analysis of the 18 predictive plasma signaling proteins. The 18 predictors identified with PAM were used for Alzheimer's (AD) and non-Alzheimer's class prediction in the training set (FIG. 4A), the blinded test set ‘AD’ (FIG. 4B) and the test set ‘MCI’ (FIG. 4C).

FIGS. 5A and 5B show functional profiling of the 18 predictors using a Direct Acyclic Graph (DAG) generated in WebGestalt (FIG. 5A) and DAVID (FIG. 5B).

FIG. 6A shows examples of filter based, arrayed sandwich ELISAs showing differences in plasma concentration of cellular signaling proteins associated with AD in sample donors with the indicated diagnoses.

FIG. 6B shows an example of the correlation of array data with ELISA data.

FIGS. 7A and 7B show patterns of signaling protein expression in Alzheimer disease compared with non-demented controls, other neurological disorders, and rheumatoid arthritis. Normalized and Z scored array measurements of 18 differentially expressed signaling proteins in plasma from subjects with Alzheimer disease (AD) and non-demented controls (NDC) are shown in a node map after unsupervised clustering (FIG. 7A). Normalized and Z scored array measurements of the 18 predictors in plasma samples from subjects with Alzheimer disease (AD), other neurological diseases (OND), or rheumatoid arthritis (RA) are shown in a node map after unsupervised clustering (FIG. 7B).

FIGS. 8A and 8B show the result of a PubMed query for additional functional annotations and biological processes of the 18 signaling proteins.

FIGS. 9-12 show sample correlations for SearchLight concentration data for related samples (FIG. 9), identical replicates (FIGS. 10 and 11), and unrelated samples (FIG. 12).

FIGS. 13-28 show histograms of SearchLight concentration data for each measured cytokine.

FIGS. 29-32 show correlations for Natural Log-transformed SearchLight concentration data.

FIGS. 33A-33C show ELISA results for 3 proteins, FIG. 33A BDNF; FIG. 33B Leptin; and FIG. 33C RANTES, selected from the list from Table 10 shown herein in the Examples. 95 plasma samples from individuals having AD and having mean MMSE scores of 20, and mean age of 74, was compared to plasma sample from 88 age-matched controls having mean MMSE score of 30. Non-parametric, unpaired t tests comparing the mean concentration of each protein was used to determine statistical significance (p-value).

FIG. 34 shows a Cell Bar Chart for concentration of BDNF in plasma. (Cell Bar Chart Grouping Variable(s): stage Error Bars: ÷1 Standard error(s) Inclusion criteria: Sparks from Center All)

FIG. 35 shows BDNF in control vs AD for male and female. (Cell Bar Chart Grouping Variable(s): Disease Split By: sex Error Bars: ±1 Standard Error(s) Row exclusion: Center All)

FIG. 36 shows RANTES concentration in plasma. (Cell Bar Chart Grouping Variable(s): stage Error Bars: ±1 Standard Error(s) Row exclusion: Center All)

FIG. 37 shows concentration of Leptin in plasma. (Cell Bar Chart Grouping Variable(s): stage Error Bars: ±1 Standard Error(s) Row exclusion: Center All)

FIG. 38 shows PDGF-BB concentration in plasma. (Cell Bar Chart Grouping Variable(s): stage Error Bars: ±1 Standard Error(s) Row exclusion: Center All)

FIG. 39 shows BDNF concentration in plasma. (Cell Bar Chart Grouping Variable(s): stage Error Bars: ±1 Standard Error(s) Row exclusion: Center All)

### DETAILED DESCRIPTION OF THE INVENTION

Inflammation and injury responses are invariably associated with neuron degeneration in AD, Parkinson's Disease (PD), frontotemporal dementia, cerebrovascular disease, multiple sclerosis, and neuropathies. The brain and CNS are not only immunologically active in their own accord, but also have complex peripheral immunologic interactions. Fiala et al. (1998 Mol Med. July; 4(7):480-9) has shown that in Alzheimer's disease, alterations in the permeability of the blood-brain barrier and chemotaxis, in part mediated by chemokines and cytokines, may permit the recruitment and transendothelial passage of peripheral cells into the brain parenchyma. A paradigm of the blood-brain barrier was constructed utilizing human brain endothelial and astroglial cells with the anatomical and physiological characteristics observed in vivo. This model was used to test the ability of monocytes/macrophages to transmigrate when challenged by A beta 1-42 on the brain side of the blood-brain barrier model. In that model A beta 1-42 and monocytes on the brain side potentiated monocyte transmigration from the blood side to the brain side. In some individuals, circulating monocytes/macrophages, when recruited by chemokines produced by activated microglia and macrophages, could add to the inflammatory destruction of the brain in Alzheimer's disease.

The inventors assert that the monitoring for relative concentrations of many secreted markers measured simultaneously in the serum is a more sensitive method for monitoring the progression of disease than the absolute concentration of any single biochemical markers have been able to achieve. A composite or array embodying the use of the sets of biomarkers as described herein simultaneously, consisting of e.g. antibodies bound to a solid support or protein bound to a solid support, for the detection of inflammation and injury response markers associated with AD.

The inventors have discovered sets of biochemical markers (collectively termed “AD biomarkers”) useful for diagnosis of AD, aiding in diagnosis of AD, monitoring AD in AD patients (e.g., tracking disease progression in AD patients, which may be useful for tracking the effect of medical or surgical therapy in AD patients). The AD biomarkers are present in biological fluids of individuals. In some examples, the AD biomarkers are present in peripheral biological fluids (e.g., blood) of individuals, allowing collection of samples by procedures that are relatively non-invasive, particularly as compared to the lumbar puncture procedure commonly used to collect cerebrospinal fluid samples.

### DEFINITIONS

As used herein, the terms “Alzheimer's patient”, “AD patient”, and “individual diagnosed with AD” all refer to an individual who has been diagnosed with AD or has been given a probable diagnosis of Alzheimer's Disease (AD).

As used herein, the phrase “AD biomarker” refers to a biomarker that is an AD diagnosis biomarker.

The term “AD biomarker polynucleotide”, as used herein, refers to any of: a polynucleotide sequence encoding a AD biomarker, the associated trans-acting control elements (e.g., promoter, enhancer, and other gene regulatory sequences), and/or mRNA encoding the AD biomarker.

As used herein, methods for “aiding diagnosis” refer to methods that assist in making a clinical determination regarding the presence, or nature, of the AD, and may or may not be conclusive with respect to the definitive diagnosis. Accordingly, for example, a method of aiding diagnosis of AD can comprise measuring the amount of the AD biomarkers in a biological sample from an individual.

As used herein, the term “predicting” refers to making a finding that an individual has a significantly enhanced probability of developing AD.

As used herein, “biological fluid sample” encompasses a variety of fluid sample types obtained from an individual and can be used in a diagnostic or monitoring assay. The definition encompasses blood, cerebral spinal fluid (CSF), urine and other liquid samples of biological origin. The definition also includes samples that have been manipulated in any way after their procurement, such as by treatment with reagents, solubilization, or enrichment for certain components, such as proteins or polynucleotides.

As used herein, the term “peripheral biological fluid sample” refers to a biological fluid sample that is not derived from the central nervous system (i.e., is not a CSF sample) and includes blood samples and other biological fluids not derived from the CNS.

A “blood sample” is a biological sample which is derived from blood, preferably peripheral (or circulating) blood. A blood sample may be, for example, whole blood, plasma or serum.

An “individual” is a mammal, more preferably a human. Mammals include, but are not limited to, humans, primates, farm animals, sport animals, rodents and pets.

A “Normal” individual or sample from a “Normal” individual as used herein for quantitative and qualitative data refers to an individual who has or would be assessed by a physician as not having AD, and has an Mini-Mental State Examination (MMSE) (referenced in Folstein et al., J. Psychiatr. Res 1975; 12:1289-198) score or would achieve a MMSE score in the range of 25-30. A “Normal” individual is generally age-matched within a range of 5 to 10 years, including but not limited to an individual that is age-matched, with the individual to be assessed.

In general, an individual with “Questionable AD” as used herein is an individual who (a) has been diagnosed with AD or has been given a diagnosis of probable AD, and (b) has either been assessed with the Mini-Mental State Examination (MMSE) (referenced in Folstein et al., J. Psychiatr. Res 1975; 12:1289-198) and scored 25-28 or would achieve a score of 25-28 upon MMSE testing. Accordingly, “Questionable AD” refers to AD in a individual having scored 25-28 on the MMSE and or would achieve a score of 25-28 upon MMSE testing.

In general, an “individual with mild AD” is an individual who (a) has been diagnosed with AD or has been given a diagnosis of probable AD, and (b) has either been assessed with the Mini-Mental State Examination (MMSE) (referenced in Folstein et al., J. Psychiatr. Res 1975; 12:1289-198) and scored 22-27 or would achieve a score of 22-27 upon MMSE testing. Accordingly, “mild AD” refers to AD in a individual who has either been assessed with the MMSE and scored 22-27 or would achieve a score of 22-27 upon MMSE testing. In some embodiments, the MMSE score range for “mild AD” is 20-25.

In general, an “individual with moderate AD” is an individual who (a) has been diagnosed with AD or has been given a diagnosis of probable AD, and (b) has either been assessed with the MMSE and scored 16-21 or would achieve a score of 16-21 upon MMSE testing. Accordingly, “moderate AD” refers to AD in a individual who has either been assessed with the MMSE and scored 16-21 or would achieve a score of 16-21 upon MMSE testing. In some embodiments, the MMSE score range for “moderate AD” is 10-20.

In general, an “individual with severe AD” is an individual who (a) has been diagnosed with AD or has been given a diagnosis of probable AD, and (b) has either been assessed with the MMSE and scored 12-15 or would achieve a score of 12-15 upon MMSE testing. Accordingly, “severe AD” refers to AD in a individual who has either been assessed with the MMSE and scored 12-15 or would achieve a score of 12-15 upon MMSE testing. In some embodiments, the MMSE score range for “severe AD” is 10-20.

As used herein, the term “treatment” refers to the alleviation, amelioration, and/or stabilization of symptoms, as well as delay in progression of symptoms of a particular disorder. For example, “treatment” of AD includes any one or more of: elimination of one or more symptoms of AD, reduction of one or more symptoms of AD, stabilization of the symptoms of AD (e.g., failure to progress to more advanced stages of AD), and delay in progression (i.e., worsening) of one or more symptoms of AD.

As used herein, the phrase “fold difference” refers to a numerical representation of the magnitude difference between a measured value and a reference value for an AD biomarker. Fold difference is calculated mathematically by division of the numeric measured value with the numeric reference value. For example, if a measured value for an AD biomarker is 20 nanograms/milliliter (ng/ml), and the reference value is 10 ng/ml, the fold difference is 2 (20/10=2). Alternatively, if a measured value for an AD biomarker is 10 nanograms/milliliter (ng/ml), and the reference value is 20 ng/ml, the fold difference is 10/20 or −0.50 or −50%).

As used herein, a “reference value” can be an absolute value; a relative value; a value that has an upper and/or lower limit; a range of values; an average value; a median value, a mean value, a shrunken centroid value, or a value as compared to a particular control or baseline value. It is to be understood that other statistical variables may be used in determining the reference value. A reference value can be based on an individual sample value, such as for example, a value obtained from a sample from the individual with AD, but at an earlier point in time, or a value obtained from a sample from an AD patient other than the individual being tested, or a “normal” individual, that is an individual not diagnosed with AD. The reference value can be based on a large number of samples, such as from AD patients or normal individuals or based on a pool of samples including or excluding the sample to be tested.

As used herein, “a”, “an”, and “the” can mean singular or plural (i.e., can mean one or more) unless indicated otherwise.

### Methods of the Invention

Methods for identifying biomarkers

The sets of biomarkers for use in the methods described herein include the set: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a; and the set: MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6. In some embodiments, these sets of biomarkers do not include any additional biomarkers. In some embodiments, these sets of biomarkers may further include additional biomarkers. Accordingly, described herein are methods for identifying one or more additional biomarkers useful for diagnosis, aiding in diagnosis, assessing risk, monitoring, and/or predicting AD.

The invention provides methods for identifying one or more biomarkers useful for diagnosis, aiding in diagnosis, diagnosis, assessing risk, monitoring, and/or predicting AD. In certain aspects of the invention, levels of a group of biomarkers are obtained for a set of peripheral biological fluid samples from one or more individuals. The samples are selected such that they can be segregated into one or more subsets on the basis of AD (e.g., samples from healthy individuals, those diagnosed with other dementias or disorders (as other dementia controls), or samples from individuals with Alzheimer's disease). The measured values from the samples are compared to each other to identify those biomarkers which differ significantly amongst the subsets. Those biomarkers that vary significantly amongst the subsets may then be used in methods for aiding in the diagnosis, monitoring, and/or prediction of AD. In other aspects of the invention, measured values for a set of peripheral biological fluid samples from one or more individuals (where the samples can be segregated into one or more subsets on the basis of AD) are compared, wherein biomarkers that vary significantly are useful for aiding in the diagnosis, diagnosis, monitoring, and/or prediction of AD. In further aspects of the invention, levels of a set of peripheral biological fluid samples from one or more individuals (where the samples can be segregated into one or more subsets on the basis of a AD) are measured to produced measured values, wherein biomarkers that vary significantly are useful for aiding in the diagnosis, diagnosis, monitoring, and/or prediction of AD.

The instant invention utilizes a set of peripheral biological fluid samples, such as blood samples, that are derived from one or more individuals. The set of samples is selected such that it can be divided into one or more subsets on the basis of AD. The division into subsets can be on the basis of presence/absence of disease, or subclassification of disease (e.g., relapsing/remitting vs. progressive relapsing). Biomarkers measured in the practice of the invention may be any proteinaceous biological marker found in a peripheral biological fluid sample. Tables 14 and 15 contain a collection of exemplary biomarkers. Additional biomarkers are described herein in the Examples.

Accordingly, the invention provides methods for identifying one or more biomarkers which can be used to aid in the diagnosis, to diagnose, detect, and/or predict AD. The methods of the invention are carried out by obtaining a set of measured values for a plurality of biomarkers from a set of peripheral biological fluid samples, where the set of peripheral biological fluid samples is divisible into at least two subsets in relation to AD, comparing said measured values between the subsets for each biomarker, and identifying biomarkers which are significantly different between the subsets.

The process of comparing the measured values may be carried out by any method known in the art, including Significance Analysis of Microarrays, Tree Harvesting, CART, MARS, Self Organizing Maps, Frequent Item Set, or Bayesian networks.

In one aspect, the invention provides methods for identifying one or more biomarkers useful for the diagnosis of AD by obtaining measured values from a set of peripheral biological fluid samples for a plurality of biomarkers, wherein the set of peripheral biological fluid samples is divisible into subsets on the basis of AD, comparing the measured values from each subset for at least one biomarker; and identifying at least one biomarker for which the measured values are significantly different between the subsets. In some embodiments, the comparing process is carried out using Significance Analysis of Microarrays.

In another aspect, the invention provides methods for identifying at least one biomarker useful for aiding in the diagnosis of AD by obtaining measured values from a set of peripheral biological fluid samples for a plurality of biomarkers, wherein the set of peripheral biological fluid samples is divisible into subsets on the basis of AD, comparing the measured values from each subset for at least one biomarker; and identifying biomarkers for which the measured values are significantly different between the subsets.

In another aspect, the invention provides methods for identifying at least one biomarker useful for the monitoring of AD by obtaining measured values from a set of peripheral biological fluid samples for a plurality of biomarkers, wherein the set of peripheral biological fluid samples is divisible into subsets on the basis of strata of AD, comparing the measured values from each subset for at least one biomarker; and identifying biomarkers for which the measured values are significantly different between the subsets. In other examples, the measured values are obtained from peripheral biological fluid samples of varying sources.

In yet another aspect, the invention provides methods for identifying at least one biomarker useful for the prediction of AD by obtaining measured values from a set of peripheral biological fluid samples for a plurality of biomarkers, wherein the set of peripheral biological fluid samples is divisible into subsets on the basis of AD, comparing the measured values from each subset for at least one biomarker; and identifying biomarkers for which the measured values are significantly different between the subsets. In other examples, the measured values are obtained from peripheral biological fluid samples of varying sources.

### Methods of Assessing AD

Provided herein are methods for assessing AD, diagnosing or aiding diagnosis of AD by obtaining measured levels of sets of AD diagnosis biomarkers in a biological fluid sample from an individual, such as for example, a peripheral biological fluid sample from an individual, and comparing those measured levels to reference levels, wherein the sets of biomarkers comprise: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a; or comprise: MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6, either set of which may optionally comprise additional biomarkers (e.g. one, two, three, or more additional biomarkers). Reference to “AD diagnosis markers” “AD biomarker” and “Biomarker” (used interchangeably herein) are terms of convenience to refer to the markers described herein and their use, and is not intended to indicate the markers are only used to diagnose AD. As this disclosure makes clear, these biomarkers are useful for, for example, assessing risk of developing AD, etc. AD biomarkers include but are not limited to secreted proteins or metabolites present in a person's biological fluids (that is, a biological fluid sample), such as for example, blood, including whole blood, plasma or serum; urine; cerebrospinal fluid; tears; and saliva. Biological fluid samples encompass clinical samples, and also includes serum, plasma, and other biological fluids. A blood sample may include, for example, various cell types present in the blood including platelets, lymphocytes, polymorphonuclear cells, macrophages, erythrocytes.

As described herein, assessment of results can depend on whether the data were obtained by the qualitative or quantitative methods described herein and/or type of reference point used. For example, as described in Example 6, qualitative measurement of AD biomarker levels relative to another reference level, which may be relative to the level of another AD biomarker, may be obtained. In other methods described herein, such as in Example 9, quantitative or absolute values, that is protein concentration levels, in a biological fluid sample may be obtained. “Quantitative” result or data refers to an absolute value (see Example 9), which can include a concentration of a biomarker in pg/ml or ng/ml of molecule to sample. An example of a quantitative value is the measurement of concentration of protein levels directly for example by ELISA. “Qualitative” result or data provides a relative value which is as compared to a reference value. In some examples herein (Example 6), qualitative measurements are assessed by signal intensity on a filter. In some examples herein, multiple antibodies specific for AD biomarkers are attached to a suitable surface, e.g. as slide or filter. As described herein in Examples 12 and 13, qualitative assessment of results may include normalizing data. In this disclosure, various sets of biomarkers are described. It is understood that the invention contemplates use of any of these sets.

In one aspect, the present invention provides methods of aiding diagnosis of Alzheimer's disease (“AD”) and diagnosing AD, by obtaining measured levels of each AD diagnosis biomarker in a set of AD biomarkers in a biological fluid sample from an individual, such as for example, a peripheral biological fluid sample from an individual, and comparing those measured levels to reference levels. In some examples, a peripheral biological fluid sample is plasma. In some examples, the set of AD diagnosis biomarkers comprises: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some examples, the set of AD diagnosis biomarkers comprises: MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6. Optionally, these sets may further comprise additional biomarkers, such as those described in Tables 14-21 in Examples 3-11, Tables 16-20 of which are described in more detail below.

Tables 16A1-16A2 and 16B provide a listing of biomarkers (clustered by methods as described herein) in order of highest ranked biomarker to lowest ranked biomarker within each cluster based on score value) that are significantly increased (16A1-16A2) or decreased (16B) in AD compared to age-matched normal controls plus other non-AD forms of neurodegeneration, such as for example PD and PN (that is, as compared to all controls). Generally, a significant increase in a biomarker as compared to an appropriate control is indicative of AD, and a significant decrease in a biomarker as compared to an appropriate control is indicative of AD. The columns from left to right in Tables 16A1-16A2 and 16B are Biomarker name, Score(d); Fold change; q-value(%); and cluster number. The biomarkers listed in Tables 16A1-16A2 and 16B, that is, reagents specific for the biomarker, may be useful as additional biomarkers in the methods described herein, such as for example, for aiding in the diagnosis of or diagnosing AD, such as for example, for diagnosing AD as distinguished from other non-AD neurodegenerative diseases or disorders, such as for example PD and PN.

Tables 17A1-17A2 and 17B provide a listing of biomarkers (not clustered and in order of highest ranked biomarker to lowest ranked biomarker based on score value) that are significantly increased (17A1-17A2) or decreased (17B) in AD compared to healthy age-matched controls. The columns from left to right in Tables 17A1-17A2 and 17B, Tables 18A1-18A2 and 18B, and Tables 19A-19B are Biomarker name, Score(d); Fold change; and q-value(%). The biomarkers listed in Tables 17A1-17A2 and 17B, that is, reagents specific for the biomarker, may be useful as additional biomarkers in the methods disclosed herein, such as for example, for aiding in the diagnosis of or diagnosing AD. In some embodiments, the additional biomarker has a p-value of equal to or less than 0.05, (or a q-value (%) of equal to or less than 5.00). For Tables 17A1-17A2 (biomarkers increased or positively correlated) biomarkers GRO, GITR-Light, IGFBP, HGF, IL-1R4/ST, IL-2Ra, ENA-78, and FGF-9 have a p-value of greater than 0.05.

Tables 18A1-18A2 and 18B provide a listing of biomarkers (not clustered and in order of highest ranked biomarker to lowest ranked biomarker based on score value) that are significantly increased (18A1-18A2) or decreased (18B) in AD compared to age-matched degenerative controls. The biomarkers listed in Tables 18A1-18A2 and 18B, that is, reagents specific for the biomarker, may be useful as additional biomarkers in the methods described herein, such as for example, for aiding in the diagnosis of or diagnosing AD. In some embodiments, the additional biomarker has a p-value of equal to or less than 0.05, (or a q-value (%) of equal to or less than 5.00). In some embodiments, the additional biomarker has a p-value of greater than 0.05, (or a q-value (%) of greater than 5.00).

Tables 19A-19B provide a listing of biomarkers (not clustered and in order of highest ranked biomarker to lowest ranked biomarker based on score value) that are significantly increased (19A) or decreased (19B) in AD plus other non-AD neurodegenerative controls with reference to age matched controls. The biomarkers listed in Tables 19A-19B, that is, reagents specific for the biomarker, may be useful as additional biomarkers in the methods disclosed herein, such as for example, for aiding in the diagnosis of AD. These biomarkers may also be useful as an initial or secondary screening for neurological disease, concurrently with methods for aiding diagnosis of AD and/or diagnosing AD using the sets of biomarkers described herein.

Methods of aiding diagnosis of AD and diagnosing AD as described herein may comprise any of the following steps of obtaining a biological fluid sample from an individual, measuring the level of each AD diagnosis biomarker in the set in the sample and comparing the measured level to an appropriate reference; obtaining measured levels of each AD diagnosis biomarker in the set in a sample and comparing the measured level to an appropriate reference; comparing measured levels of each AD diagnosis biomarker in the set obtained from a sample to an appropriate reference; measuring the level of each AD diagnosis biomarker in the set in a sample; measuring the level of each AD diagnosis biomarker in the set in a sample and comparing the measured level to an appropriate reference; diagnosing AD based on comparison of measured levels to an appropriate reference; or obtaining a measured value for each AD diagnosis biomarker in the set in a sample. Comparing a measured level of an AD diagnosis biomarker to a reference level or obtaining a measured value for an AD diagnosis biomarker in a sample may be performed for each AD diagnosis biomarker in the set. The present invention also provides methods of evaluating results of the analytical methods described herein. Such evaluation generally entails reviewing such results and can assist, for example, in advising regarding clinical and/or diagnostic follow-up and/or treatment options. The present invention also provides methods for assessing a biological fluid sample for an indicator of any one or more of the following: AD; progression of AD; by measuring the level of or obtaining the measured level of or comparing a measured level of each AD diagnosis biomarker in a set of biomarkers as described herein.

Provided herein are methods for assessing the efficacy of treatment modalities in individuals, or population(s) of individuals, such as from a single or multiple collection center(s), diagnosed with AD or predicted to be at risk of converting to AD comprising any one of the following steps: obtaining a biological fluid sample from the individual(s) subject to treatment; measuring the level of each AD diagnosis biomarker in the set in the sample and comparing the measured level to an appropriate reference, which in some examples is a measured level of the biomarker in a fluid sample obtained from the individual(s) prior to treatment; obtaining measured levels of each AD diagnosis biomarker in the set in a sample from the individual(s) and comparing the measured level to an appropriate reference; comparing measured levels of each AD diagnosis biomarker in the set obtained from a sample from the individual(s) to an appropriate reference; measuring the level of each AD diagnosis biomarker in the set in a sample from the individual(s); measuring the level of each AD diagnosis biomarker in the set in a sample from the individual(s) and comparing the measured level to an appropriate reference; diagnosing efficacy of treatment based on comparison of measured levels to an appropriate reference; or obtaining a measured value for each AD diagnosis biomarker in the set in a sample. Measured levels of each AD diagnosis biomarker in the set may be obtained once or multiple times during assessment of the treatment modality.

For methods of diagnosing AD as described herein, the reference level is generally a predetermined level considered ‘normal’ for the particular AD diagnosis biomarker (e.g., an average level for age-matched individuals not diagnosed with AD or an average level for age-matched individuals diagnosed with neurological disorders other than AD and/or healthy age-matched individuals), although reference levels which are determined contemporaneously (e.g., a reference value that is derived from a pool of samples including the sample being tested) are also contemplated. Also provided are methods of aiding in the diagnosis of Alzheimer's disease (“AD”) by comparing a measured level of each AD diagnosis biomarker in the set in a biological fluid sample, such as, for example, a peripheral biological fluid sample from an individual with a reference level. Further provided are methods of aiding in the diagnosis of Alzheimer's disease (“AD”) by measuring a level of each AD diagnosis biomarker in the set in a biological fluid sample, such as, for example, a peripheral biological fluid sample from an individual. For the AD diagnosis biomarkers disclosed herein, a measurement for a marker which is below or above the reference level suggests (i.e., aids in the diagnosis of) or indicates a diagnosis of AD.

In a further aspect, the invention provides methods of monitoring progression of AD in an AD patient. For example, as shown in Example 9, the inventors have found that quantitative levels of RANTES are decreased in AD patients with Questionable AD (MMSE=25-28); and that quantitative levels of RANTES are decreased in AD patients with mild AD (MMSE=20-25), and RANTES levels decrease further as the severity of the AD intensifies. Additionally, the inventors have found that quantitative PDGF-BB levels are decreased in AD patients with Questionable AD; that PDGF-BB levels are decreased in Questionable AB compared to Mild AD; and that the MMSE scores for male AD patients are negatively correlated with PDGF-BB levels (as described in Example 9). An individual with “Questionable AD” as used herein for quantitative data (also called absolute measurement) is an individual who (a) has been diagnosed with AD or has been given a diagnosis of probable AD, and (b) has either been assessed with the Mini-Mental State Examination (MMSE) (referenced in Folstein et al., J. Psychiatr. Res 1975; 12:1289-198) and scored 25-28 or would achieve a score of 25-28 upon MMSE testing. Accordingly, “Questionable AD” refers to AD in a individual having scored 25-28 on the MMSE and or would achieve a score of 25-28 upon MMSE testing. The reference level may be a predetermined level considered ‘normal’ for the particular biomarker (e.g., an average level for age-matched and/or sex-matched individuals not diagnosed with AD), or may be a historical reference level for the particular patient (e.g., a biomarker level that was obtained from a sample derived from the same individual, but at an earlier point in time). Reference levels which are determined contemporaneously (e.g., a reference value that is derived from a pool of samples including the sample being tested) are also contemplated. Accordingly, the invention provides methods for monitoring progression of AD in an AD patient by obtaining quantitative values for each biomarker in the set from a biological fluid sample, such as for example, a peripheral biological fluid sample and comparing measured values to reference values. For example, a decrease or increase in the measured value indicates or suggests (diagnoses or suggests a diagnosis) progression (e.g., an increase in the severity) of AD in the AD patient.

An AD biomarker that stays “substantially the same” means that there is not a significant change, and that the values stay about the same. In some embodiments, substantially the same is a change less than any of about 12%, 10%, 5%, 2%, 1%. In some embodiments, a significant change means not statistically significant using standard methods in the art. The methods described above are also applicable to methods for assessing progression of AD.

The results of the comparison between the measured value(s) and the reference value(s) are used to diagnose or aid in the diagnosis of AD, or to monitor progression of AD in an AD patient. Accordingly, if the comparison indicates a difference (that is, an increase or decrease) between the measured value(s) and the reference value(s) that is suggestive/indicative of AD, then the appropriate diagnosis is aided in or made. Conversely, if the comparison of the measured level(s) to the reference level(s) does not indicate differences that suggest or indicate a diagnosis of AD, then the appropriate diagnosis is not aided in or made.

As will be understood by those of skill in the art, in the practice of the AD diagnosis methods of the invention (i.e., methods of diagnosing or aiding in the diagnosis of AD), more than one AD diagnosis biomarker is used, and the method used for evaluating a diagnosis of AD may vary. For example, in some embodiments, when the markers do not unanimously suggest or indicate a diagnosis of AD, the ‘majority’ suggestion or indication (e.g., when the method utilizes sixteen AD diagnosis biomarkers, ten of which suggest/indicate AD, the result would be considered as suggesting or indicating a diagnosis of AD for the individual) is considered the result of the assay. In some embodiments, the overall pattern of the markers (e.g. how each marker compares with one or more sets of references levels) is used in diagnosing AD. Various algorithms, classifiers, and/or decision trees as described herein may be used to evaluate the overall levels of the biomarkers to determine a diagnosis.

As will be appreciated by one of skill in the art, methods disclosed herein may include the use of any of a variety of biological markers (which may or may not be AD markers) to determine the integrity and/or characteristics of the biological sample(s) (e.g. gender).

In various embodiments, the sensitivity achieved by the use of the set of AD biomarkers in a method for diagnosing or aiding diagnosis of AD is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the specificity achieved by the use of the set of AD biomarkers in a method for diagnosing or aiding diagnosis of AD is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the overall accuracy achieved by the use of the set of AD biomarkers in a method for diagnosing or aiding diagnosis of AD is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In some embodiments, the sensitivity and/or specificity are measured against a clinical diagnosis of AD.

In certain embodiments of the invention, levels for AD biomarkers are obtained from an individual at more than one time point. Such “serial” sampling is well suited for the aspects of the invention related to monitoring progression of AD in an AD patient. Serial sampling can be performed on any desired timeline, such as monthly, quarterly (i.e., every three months), semi-annually, annually, biennially, or less frequently. The comparison between the measured levels and the reference level may be carried out each time a new sample is measured, or the data relating to levels may be held for less frequent analysis.

As will be understood by those of skill in the art, biological fluid samples including peripheral biological fluid samples are usually collected from individuals who are suspected of having AD, or developing AD. The invention also contemplates samples from individuals for whom AD diagnosis is desired. Alternatively, individuals (or others involved in for example research and/or clinicians may desire such assessments without any indication of AD, suspected AD, or risk for AD. For example, a normal individual may desire such information. Such individuals are most commonly 65 years or older, although individuals from whom biological fluid samples, such as peripheral biological fluid samples are taken for use in the methods of the invention may be as young as 35 to 40 years old, when early onset AD or familial AD is suspected.

The invention also provides methods of screening for candidate agents for the treatment of AD by assaying prospective candidate agents for activity in modulating the set of AD biomarkers. The screening assay may be performed either in vitro and/or in vivo. Candidate agents identified in the screening methods described herein may be useful as therapeutic agents for the treatment of AD.

The probability P that the composite is more predictive than any subset of markers present in the composite can be expressed mathematically as:

P=1−(1−P₁)(1−P₂)(1−P₃) . . . (1−Pₙ)

Where the probability P₁, P₂, Pₙ represent the probability of individual marker being able to predict clinical phenotypes, and where 1-Pₙ represents the complement of that probability. Any subset of the composite, will always therefore have a smaller value for P.

In accordance with a further embodiment of the present invention, the relative concentrations in serum, CSF, or other fluids of the biomarkers MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a as a composite, or collective, optionally further comprising the biomarkers: TRAIL R4 and IGFBP-6, and/or optionally further comprising additional biomarkers is more predictive than the absolute concentration of any individual marker in predicting clinical phenotypes, disease detection, monitoring, and treatment of AD. In some embodiments, the composite group of biomarkers is MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, the composite group of biomarkers is MCSF, RANTES, GCSF, PARC, ANG-2, IL-11, EGF, MCP-3, IL-3, MIP-1delta, ICAM-1, PDGF-BB, IL-8, GDNF, IL-1a, TNF-a, TRAIL R4, and IGFBP-6.

AD diagnosis Biomarkers

Immune mechanisms are an essential part of the host defense system and typically feature prominently in the inflammatory response. A growing number of studies are discovering intriguing links between the immune system and the CNS. For example, it has become clear that the CNS is not entirely sheltered from immune surveillance and that various immune cells can traverse the blood-brain barrier. Invading leukocytes can attack target antigens in the CNS or produce growth factors that might protect neurons against degeneration (Hohlfeld et al., 2000, J. Neuroimmunol. 107, 161-166). These responses are elicited through a variety of protein mediators, including but not limited to cytokines, chemokines, neurotrophic factors, collectins, kinins, and acute phase proteins in the immune and inflammatory systems, in intercellular communication across neurons, glial cells, endothelial cells and leukocytes. Without being bound by theory, it is hypothesized that the cytokines, chemokines, neurotrophic factors, collectins, kinins, and acute phase proteins listed herein are differentially expressed in serum associated with neurodegenerative and inflammatory diseases such as Alzheimer's. Cytokines are a heterogeneous group of polypeptide mediators that have been associated with activation of numerous functions, including the immune system and inflammatory responses. Peripheral cytokines also penetrate the blood-brain barrier directly via active transport mechanisms or indirectly via vagal nerve stimulation. Cytokines can act in an autocrine manner, affecting the behavior of the cell that releases the cytokine, or in a paracrine manner, affecting the behavior of adjacent cells. Some cytokines can act in an endocrine manner, affecting the behavior of distant cells, although this depends on their ability to enter the circulation and on their half-life. The cytokine families include, but are not limited to, interleukins (IL-1 alpha, IL-1 beta, ILIra and IL-2 to IL-18), tumor necrosis factors (TNF-alpha and TNF-beta), interferons (INF-alpha, beta and gamma), colony stimulating factors (G-CSF, M-CSF, GM-CSF, IL-3 and some of the other ILs), and growth factors (EGF, FGF, PDGF, TGF alpha, TGF betas, BMPs, GDFs, CTGF, and ECGF).

The inventors have discovered a collection of biochemical markers present in peripheral bodily fluids that may be used to assess AD, including diagnose or aid in the diagnosis of AD. This group of AD diagnosis markers comprises: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a, and may optionally further comprise: TRAIL R4 and IGFBP-6.

The AD diagnosis biomarkers discovered by the inventors are all known molecules. Brain derived neurotrophic factor (BDNF) is described in, for example Rosenthal et al., 1991, Endocrinology 129(3):1289-94. Basic fibroblast growth factor (bFGF) is described in, for example Abraham et al., 1986, EMBO J. 5(10):2523-28. Epidermal growth factor (EGF) is described in, for example Gray et al., 1983, Nature 303(5919):722-25. Fibroblast growth factor 6 (FGF-6) is described in, for example Marics et al., 1989, Oncogene 4(3):335-40. Interleukin-3 (IL-3) is described in, for example Yang et al., 1986, Cell 47(1):3-10. Soluble interleukin-6 receptor (sIL-6R) is described in, for example, Taga et al., 1989, Cell 58(3):573-81. Leptin (also known as “ob”) is described in, for example Masuzaki et al. 1995, Diabetes 44(7):855-58. Macrophage inflammatory protein-1 delta (MIP-16) is described in, for example Wang et al., 1998, J. Clin. Immunol. 18(3):214-22. Macrophage stimulating protein alpha chain (MSP-α) is described in, for example, Yoshimura et al., 1993, J. Biol. Chem. 268 (21), 15461-68, and Yoshikawa et al., 1999, Arch. Biochem. Biophys. 363(2):356-60. Neutrophil activating peptide-2 (NAP-2) is described in, for example Walz et al., 1991, Adv. Exp. Med. Biol. 305:39-46. Neurotrophin-3 (NT-3) is described in, for example Hohn et al., 1990, Nature 344(6264):339-41. BB homodimeric platelet derived growth factor (PDGF-BB) is described in, for example Collins et al., 1985, Nature 316(6030):748-50. RANTES is described in, for example Schall et al., 1988, J. Immunol. 141(3):1018-25. Stem cell factor (SCF) is described in, for example Zseboet al., 1990, Cell 63(1):213-24. Soluble tumor necrosis factor receptor-2 (sTNF R11) is described in, for example Schall et al., 1990, Cell 61(2):361-70. Transforming growth factor-beta 3 (TGF-β3) is described in, for example ten Dijke et al., 1988, Proc. Natl. Acad. Sci. U.S.A. 85 (13):4715-19. Tissue inhibitor of metalloproteases-1 (TIMP-1) is described in, for example, Docherty et al., 1985, Nature 318(6041):66-69 and Gasson et al., 1985, Nature 315(6022):768-71. Tissue inhibitor of metalloproteases-2 (TIMP-2) is described in, for example, Stetler-Stevenson et al., 1190, J. Biol. Chem. 265(23):13933-38. Tumor necrosis factor beta (TNF-β) is described in, for example Gray et al., 1984, Nature 312(5996):721-24. Thrombopoietin (TPO) is described in, for example, Foster et al., 1994, Proc. Natl. Acad. Sci. U.S.A. 91(26):13023-27.

The effectiveness (e.g., sensitivity and/or specificity) of the methods of the AD diagnosis methods of the instant invention utilizing sets of biomarkers as described herein are generally enhanced over the use of a single biomarker.

Additional AD diagnosis biomarkers may be selected from the AD diagnosis biomarkers disclosed herein by a variety of methods, including “q value” and/or by selecting for cluster diversity. Additional AD diagnosis biomarkers may be selected on the basis of “q value”, a statistical value that the inventors derived when identifying the AD diagnosis biomarkers (see Table 10 in Example 3). “q values” for selection of AD diagnosis biomarkers range from 0 to about 0.05, for example, range from less than about 0.0001 to about 0.05, and in some examples, range from about 0.01 to about 0.05. Alternately (or additionally), additional AD diagnosis biomarkers may be selected to preserve cluster diversity of selected proteins or sample diversity. The inventors have separated the AD diagnosis biomarkers into a number of clusters (see Table 1). Additional clusters of AD diagnosis markers are found in Tables 16A1-16A2 and 16B. Here the clusters are formed by qualitative measurements for each biomarker which are most closely correlated. As used herein, “correlate” or “correlation” is a simultaneous change in value of two numerically valued random variables such as MMSE scores and quantitative protein concentrations or qualitative protein concentrations. As used herein “discriminate” or “discriminatory” is refers to the quantitative or qualitative difference between two or more samples for a given variable. The cluster next to such a cluster is a cluster that is most closely correlated with the cluster. The correlations between biomarkers and between clusters can represented by a hierarchical tree generated by unsupervised clustering using a public web based software called wCLUTO available at: cluto.ccgb.umn.edu/cgi-bin/wCluto/wCluto.cgi. If more than one additional AD diagnosis biomarker is selected for testing, in some examples, the AD diagnosis biomarkers selected are at least partially diverse (i.e., the AD diagnosis biomarkers represent at least two different clusters, for example, a biomarker from cluster 4 in Table 1 and a biomarker from cluster 3 of Table 1), and in some instances the additional AD diagnosis biomarkers are completely diverse (i.e. no two of the selected AD diagnosis biomarkers are from the same cluster). Accordingly, the invention provides a number of different embodiments for diagnosing or aiding in the diagnosis of AD.

TABLE 1

| Cluster | Biomarker |
|---|---|
| 0 | bFGF |
| 1 | TPO |
| 2 | FGF-6 |
| | IL-3 |
| | sIL-6 R |
| | MIP-1d |
| | sTNF RII |
| | TNF-b |
| 3 | RANTES |
| | TIMP-1 |
| | TIMP-2 |
| 4 | BDNF |
| | EGF |
| | LEPTIN(OB) |
| | MSP-α |
| | NAP-2 |
| | NT-3 |
| | PDGF-BB |
| | SCF |
| | TGF-b3 |

### Measuring Levels of AD Biomarkers

There are a number of statistical tests for identifying biomarkers which vary significantly between the subsets, including the conventional t test. However, as the number of biomarkers measured increases, it is generally advantageous to use a more sophisticated technique, such as SAM (see Tusher et al., 2001, Proc. Natl. Acad. Sci. U.S.A. 98(9):5116-21) or Prediction Analysis of Microarray (PAM) (http://www-statstanford.edu/˜tibs/PAM/index.html). Other useful techniques include Tree Harvesting (Hastie et al., Genome Biology 2001, 2: research0003.1-0003.12), Self Organizing Maps (Kohonen, 1982b, Biological Cybernetics 43(1):59-69), Frequent Item Set (Agrawal et al., 1993 “Mining association rules between sets of items in large databases.” In Proc. of the ACM SIGMOD Conference on Management of Data, pages 207-216, Washington, D.C., May 1993), Bayesian networks (Gottardo, Statistical analysis of microarray data, A Bayesian approach. Biostatistics (2001),1,1, pp 1-37), and the commercially available software packages CART and MARS. Other statistical classifiers include SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier.

The SAM technique assigns a score to each biomarker on the basis of change in expression relative to the standard deviation of repeated measurements. For biomarkers with scores greater than an adjustable threshold, the algorithm uses permutations of the repeated measurements to estimate the probability that a particular biomarker has been identified by chance (calculated as a “q-value”), or a false positive rate which is used to measure accuracy. The SAM technique can be carried out using publicly available software called Significance Analysis of Microarrays (see www-stat class.stanford.edu/˜tibs/clickwrap/sam.html).

A biomarker is considered “identified” as being useful for aiding in the diagnosis, diagnosis, monitoring, and/or prediction of AD when it is significantly different between the subsets of peripheral biological samples tested. Levels of a biomarker are “significantly different” when the probability that the particular biomarker has been identified by chance is less than a predetermined value. The method of calculating such probability will depend on the exact method utilized to compare the levels between the subsets (e.g., if SAM is used, the q-value will give the probability of misidentification, and the p value will give the probability if the t test (or similar statistical analysis) is used). As will be understood by those in the art, the predetermined value will vary depending on the number of biomarkers measured per sample and the number of samples utilized. Accordingly, predetermined value may range from as high as 50% to as low as 20, 10, 5, 3, 2, or 1%.

As described herein, the levels of a set of AD diagnosis biomarkers are measured in a biological sample from an individual. The AD biomarker levels may be measured using any available measurement technology that is capable of specifically determining the levels of the AD biomarkers in a biological sample. The measurement may be either quantitative or qualitative, so long as the measurement is capable of indicating whether the level of each AD biomarker in the peripheral biological fluid sample is above or below the reference value for that biomarker.

The measured level may be a primary measurement of the level a particular biomarker a measurement of the quantity of biomarker itself (quantitative data, such as in Example 9), such as by detecting the number of biomarker molecules in the sample) or it may be a secondary measurement of the biomarker (a measurement from which the quantity of the biomarker can be but not necessarily deduced (qualitative data, such as Example 6), such as a measure of enzymatic activity (when the biomarker is an enzyme) or a measure of mRNA coding for the biomarker). Qualitative data may also be derived or obtained from primary measurements.

Although some assay formats will allow testing of peripheral biological fluid samples without prior processing of the sample, it is expected that most peripheral biological fluid samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood or other standard means of protein separation, enrichment, or purification. In some examples, the peripheral biological fluid sample is collected in a container comprising EDTA. See Example 14 for additional sample collection procedures. Commonly, AD biomarker levels will be measured using an affinity-based measurement technology. “Affinity” as relates to an antibody is a term well understood in the art and means the extent, or strength, of binding of antibody to the binding partner, such as an AD diagnosis biomarker as described herein (or epitope thereof). Affinity may be measured and/or expressed in a number of ways known in the art, including, but not limited to, equilibrium dissociation constant (K_{D} or K_{d}), apparent equilibrium dissociation constant (K_{D}' or K_{d}'), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope. Values of Kᵣ; reported herein in terms of mg IgG per ml or mg/ml indicate mg Ig per ml of serum, although plasma can be used.

Affinity-based measurement technology utilizes a molecule that specifically binds to the AD biomarker being measured (an “affinity reagent,” such as an antibody or aptamer), although other technologies, such as spectroscopy-based technologies (e.g., matrix-assisted laser desorption ionization-time of flight, or MALDI-TOF, spectroscopy) or assays measuring bioactivity (e.g., assays measuring mitogenicity of growth factors) may be used.

Affinity-based technologies include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

If immunoassay technology is employed, any immunoassay technology which can quantitatively or qualitatively measure the level of a AD biomarker in a biological sample may be used. Suitable immunoassay technology includes radioimmunoassay, immunofluorescent assay, enzyme immunoassay, chemiluminescent assay, ELISA, immuno-PCR, immuno-infrared, and western blot assay.

Likewise, aptamer-based assays which can quantitatively or qualitatively measure the level of a AD biomarker in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

A wide variety of affinity-based assays are known in the art. Affinity-based assays will utilize at least one epitope derived from the AD biomarker of interest, and many affinity-based assay formats utilize more than one epitope (e.g., two or more epitopes are involved in “sandwich” format assays; at least one epitope is used to capture the marker, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a single phase) and/or utilize or immunoprecipitation. Most assays involve the use of labeled affinity reagent (e.g., antibody, polypeptide, or aptamer); the labels may be, for example, enzymatic, fluorescent, chemiluminescent, radioactive, or dye molecules. In another approach, all the proteins in the biological sample can be labeled using standard protein chemistry techniques and the labeled biomarkers are captured by the affinity reagents arrayed on a solid support. Assays which amplify the signals from the probe are also known; examples of which are assays which utilize biotin and avidin, and enzyme-labeled and mediated immunoassays, such as ELISA and ELONA assays. Herein, the examples referred to as “quantitative data” the biomarker concentrations were obtained using ELISA. Either of the biomarker or reagent specific for the biomarker can be attached to a surface and levels can be measured directly or indirectly.

In a heterogeneous format, the assay utilizes two phases (typically aqueous liquid and solid). Typically an AD biomarker-specific affinity reagent is bound to a solid support to facilitate separation of the AD biomarker from the bulk of the biological sample. After reaction for a time sufficient to allow for formation of affinity reagent/AD biomarker complexes, the solid support or surface containing the antibody is typically washed prior to detection of bound polypeptides. The affinity reagent in the assay for measurement of AD biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass, Protein A beads, magnetic beads, and electrodes. Both standard and competitive formats for these assays are known in the art. Accordingly, provided herein are complexes comprising a set of AD diagnosis biomarkers as described herein bound to reagents specific for the biomarkers, wherein said reagents are attached to a surface. Also provided herein are complexes comprising a set of AD diagnosis biomarkers as described herein bound to reagents specific for the biomarkers, wherein said biomarkers are attached to a surface.

Array-type heterogeneous assays are suitable for measuring levels of AD biomarkers as the methods of the invention utilize multiple AD biomarkers. Array-type assays used in the practice of the methods of the invention will commonly utilize a solid substrate with two or more capture reagents specific for different AD biomarkers bound to the substrate a predetermined pattern (e.g., a grid). The peripheral biological fluid sample is applied to the substrate and AD biomarkers in the sample are bound by the capture reagents. After removal of the sample (and appropriate washing), the bound AD biomarkers are detected using a mixture of appropriate detection reagents that specifically bind the various AD biomarkers. Binding of the detection reagent is commonly accomplished using a visual system, such as a fluorescent dye-based system. Because the capture reagents are arranged on the substrate in a predetermined pattern, array-type assays provide the advantage of detection of multiple AD biomarkers without the need for a multiplexed detection system.

In a homogeneous format the assay takes place in single phase (e.g., aqueous liquid phase). Typically, the biological sample is incubated with an affinity reagent specific for the AD biomarker in solution. For example, it may be under conditions that will precipitate any affinity reagent/antibody complexes which are formed. Both standard and competitive formats for these assays are known in the art.

In a standard (direct reaction) format, the level of AD biomarker/affinity reagent complex is directly monitored. This may be accomplished by, for example, determining the amount of a labeled detection reagent that forms is bound to AD biomarker/affinity reagent complexes. In a competitive format, the amount of AD biomarker in the sample is deduced by monitoring the competitive effect on the binding of a known amount of labeled AD biomarker (or other competing ligand) in the complex. Amounts of binding or complex formation can be determined either qualitatively or quantitatively.

In some embodiments, sandwich antibody arrays are used in the methods of the invention. In some embodiments, a high sensitivity multiplex sandwich ELISA is used in the methods of the invention, for example, the SearchLight platform utilizing a chemiluminescent readout. In some embodiments, the SearchLight platform as described in Example 2 is used in the methods of the invention. In some embodiments, the high sensitivity multiplex sandwich ELISA platform (e.g. SearchLight platform) is used to analyze the biomarkers: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, a glass array platform that utilizes indirect fluorescence detection is used to analyze one or more biomarkers. In some of these embodiments, the one or more biomarkers analyzed with the glass array platform that utilizes indirect fluorescence detection are control biomarkers. In some embodiments, the glass array platform that utilizes indirect fluorescence detection is used to analyze the biomarkers: TRAIL R4, and IGFBP-6. In some embodiments, the Luminex platform is used in the methods of the invention.

The methods described in this patent may be implemented using any device capable of implementing the methods. Examples of devices that may be used include but are not limited to electronic computational devices, including computers of all types. When the methods described in this patent are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, world wide web, an intranet, or other network.

In one example, the methods described in this patent may be implemented in a system comprising a processor and a computer readable medium that includes program code means for causing the system to carry out the steps of the methods described in this patent. The processor may be any processor capable of carrying out the operations needed for implementation of the methods. The program code means may be any code that when implemented in the system can cause the system to carry out the steps of the methods described in this patent. Examples of program code means include but are not limited to instructions to carry out the methods described in this patent written in a high level computer language such as C++, Java, or Fortran; instructions to carry out the methods described in this patent written in a low level computer language such as assembly language; or instructions to carry out the methods described in this patent in a computer executable form such as compiled and linked machine language.

Complexes formed comprising AD biomarker and an affinity reagent are detected by any of a number of known techniques known in the art, depending on the format of the assay and the preference of the user. For example, unlabelled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye- (fluorescent or visible), bead-, or enzyme-labeled affinity reagent) or indirectly (as for affinity reagents “tagged” with biotin, expression tags, and the like). Herein the examples provided referred to as “qualitative data” filter based antibody arrays using chemiluminesense were used to obtain measurements for biomarkers.

As will be understood by those of skill in the art, the mode of detection of the signal will depend on the exact detection system utilized in the assay. For example, if a radiolabeled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

The biological sample may be divided into a number of aliquots, with separate aliquots used to measure different AD biomarkers (although division of the biological sample into multiple aliquots to allow multiple determinations of the levels of the AD biomarkers in a particular sample are also contemplated). Alternately the biological sample (or an aliquot therefrom) may be tested to determine the levels of multiple AD biomarkers in a single reaction using an assay capable of measuring the individual levels of different AD biomarkers in a single assay, such as an array-type assay or assay utilizing multiplexed detection technology (e.g., an assay utilizing detection reagents labeled with different fluorescent dye markers).

It is common in the art to perform ‘replicate’ measurements when measuring biomarkers. Replicate measurements are ordinarily obtained by splitting a sample into multiple aliquots, and separately measuring the biomarker(s) in separate reactions of the same assay system. Replicate measurements are not necessary to the methods of the invention, but many embodiments of the invention will utilize replicate testing, particularly duplicate and triplicate testing.

### Reference Levels

The reference levels used for comparison with the measured levels for the AD biomarkers may vary, depending on the aspect of the invention being practiced, as will be understood from the foregoing discussion. For AD diagnosis methods, the “reference level” is typically a predetermined reference level, such as an average of levels obtained from a population that is not afflicted with AD, but in some instances, the reference level can be a mean or median level from a group of individuals including AD patients. In some instances, the predetermined reference level is derived from (e.g., is the mean or median of) levels obtained from an age-matched population. In some examples disclosed herein, the age-matched population comprises individuals with non-AD neurodegenerative disorders. See Examples 12 and 13.

For AD monitoring methods (e.g., methods of diagnosing or aiding in the diagnosis of AD progression in an AD patient), the reference level may be a predetermined level, such as an average of levels obtained from a population that is not afflicted with AD, a population that has been diagnosed with AD, and, in some instances, the reference level can be a mean or median level from a group of individuals including AD patients. Alternately, the reference level may be a historical reference level for the particular patient (e.g., an EGF level that was obtained from a sample derived from the same individual, but at an earlier point in time). In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

### Comparing Levels of AD Biomarkers

The process of comparing a measured value and a reference value can be carried out in any convenient manner appropriate to the type of measured value and reference value for the AD biomarker at issue. As discussed above, ‘measuring’ can be performed using quantitative or qualitative measurement techniques, and the mode of comparing a measured value and a reference value can vary depending on the measurement technology employed. For example, when a qualitative colorimetric assay is used to measure AD biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values (e.g., quantitative measurements of concentration, such as nanograms of AD biomarker per milliliter of sample, or absolute amount). In other examples, measured values are qualitative. As with qualitative measurements, the comparison can be made by inspecting the numerical data, by inspecting representations of the data (e.g., inspecting graphical representations such as bar or line graphs).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for an AD biomarker. Alternately, a separate device (e.g., a digital computer) may be used to compare the measured value(s) and the reference value(s). Automated devices for comparison may include stored reference values for the AD biomarker(s) being measured, or they may compare the measured value(s) with reference values that are derived from contemporaneously measured reference samples.

In some embodiments, the methods of the invention utilize ‘simple’ or ‘binary’ comparison between the measured level(s) and the reference level(s) (e.g., the comparison between a measured level and a reference level determines whether the measured level is higher or lower than the reference level). For example, for AD diagnosis biomarkers, a comparison showing that the measured value for the biomarker is lower than the reference value indicates or suggests a diagnosis of AD. For example, for AD diagnosis biomarkers, a comparison showing that the measured value for the biomarker is higher than the reference value indicates or suggests a diagnosis of AD.

Various algorithms and classifiers may also be used in formulating groups of predictive markers, classification decision trees, and/or comparing the measured biomarkers levels with the reference level(s). Classifiers which may be used in the methods of the invention, include, but are not limited to: PAM, SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier.

In some embodiments, Prediction Analysis of Microarray (PAM) (http://www-stat.stanford.edu/˜tibs/PAM/index.html and http://www-stat.stanford.edu/—tibs/PAM/Rdist/howwork.html) is used for classifying a sample. One embodiment for use of PAM is briefly described.

Briefly, the method computes a standardized centroid for each class. This is the average gene expression for each gene in each class divided by the within-class standard deviation for that gene. Nearest centroid classification takes the gene expression profile of a new sample, and compares it to each of these class centroids. The class whose centroid that it is closest to, in squared distance, is the predicted class for that new sample. Nearest shrunken centroid classification makes an important modification to standard nearest centroid classification. It “shrinks” each of the class centroids toward the overall centroid for all classes by an amount called the threshold. This shrinkage consists of moving the centroid towards zero by threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero. After shrinking the centroids, the new sample is classified by the usual nearest centroid rule, but using the shrunken class centroids. This shrinkage has two advantages: 1) it can make the classifier more accurate by reducing the effect of noisy genes, 2) it does automatic gene selection. In particular, if a gene is shrunk to zero for all classes, then it is eliminated from the prediction rule. Alternatively, it may be set to zero for all classes except one, and we learn that high or low expression for that gene characterizes that class.

The user decides on the value to use for threshold. Typically one examines a number of different choices. To guide in this choice, PAM does K-fold cross-validation for a range of threshold values. The samples are divided up at random into K roughly equally sized parts. For each part in turn, the classifier is built on the other K−1 parts then tested on the remaining part. This is done for a range of threshold values, and the cross-validated misclassification error rate is reported for each threshold value. Typically, the user would choose the threshold value giving the minimum cross-validated misclassification error rate. What one gets from this is a (typically) accurate classifier, that is simple to understand.

In some embodiments, the following method may be used: A semi-supervised prediction analysis is performed using the statistical package PAM 2.3.1 with the statistical tool R. PAM executes a sample classification training routine from expression data via the nearest shrunken centroid procedure to find markers that discriminate best between two classes (e.g. AD vs non-demented controls (NDC)). It is to be understood that other classes (e.g. other dementia (OD)) may be used in the training set. Then an internal cross-validation is applied by 10-times randomly selecting 90% of the training samples in a class-balanced way to predict each time the class labels on the remaining 10% of samples (10-fold cross-validation). This assesses and minimizes classification errors and avoids over fitting. The obtained minimal number of predictors/markers is then used for a heterogeneity analysis to perform two-class prediction in a test set between a diseased group and a control group. In an example where AD and NDC are used as the two classes in the training set, if the test set contains any class other than AD or NDC (e.g. OD), PAM would then classify these OD samples as Non-AD (as there was no OD used in the training set).

As described herein, biological fluid samples may be measured quantitatively (absolute values) or qualitatively (relative values). The respective AD biomarker levels for a given assessment may or may not overlap.

In certain aspects of the invention, the comparison is performed to determine the magnitude of the difference between the measured and reference values (e.g., comparing the ‘fold’ or percentage difference between the measured value and the reference value). A fold difference that is about equal to or greater than the minimum fold difference disclosed herein suggests or indicates a diagnosis of AD, conversion from MCI to AD, or prediction of conversion from MCI to AD, as appropriate to the particular method being practiced. A fold difference can be determined by measuring the absolute concentration of a protein and comparing that to the absolute value of a reference, or a fold difference can be measured by the relative difference between a reference value and a sample value, where neither value is a measure of absolute concentration, and/or where both values are measured simultaneously. A fold difference can be in the range of 10% to 95%. An ELISA measures the absolute content or concentration of a protein from which a fold change is determined in comparison to the absolute concentration of the same protein in the reference. An antibody array measures the relative concentration from which a fold change is determined. Accordingly, the magnitude of the difference between the measured value and the reference value that suggests or indicates a particular diagnosis will depend on the particular AD biomarker being measured to produce the measured value and the reference value used (which in turn depends on the method being practiced). Tables 2A-2B list minimum fold difference values for particular AD biomarkers for use in some embodiments of the methods of the invention (either as part of a set of sixteen or eighteen biomarkers as described herein, or as an additional biomarker to the sets) which utilize a fold difference in making the comparison between the measured value and the reference value. In those embodiments utilizing fold difference values, a fold difference of about the fold difference indicated in Table 2A suggests a diagnosis of AD, wherein the fold change is a negative value. For example, a fold change of −46% for a particular biomarker means a reduction of that biomarker level by 46%. As shown in Table 2A, for qualitative measurements using antibodies, a biomarker fold change of 0.60 means a reduction in that biomarker level by about 60%. Table 2B provides additional information regarding fold changes.

TABLE 2A

| | Fold Change (as |
|---|---|
| | negative value or |
| Biomarker | decrease) |
| BDNF | 0.60 |
| bFGF | 0.75 |
| EGF | 0.60 |
| FGF-6 | 0.70 |
| IL-3 | 0.80 |
| sIL-6 R | 0.75 |
| Leptin | 0.55 |
| MIP-1δ | 0.60 |
| MSP-α | 0.80 |
| NAP-2 | 0.75 |
| NT-3 | 0.75 |
| PDGF-BB | 0.60 |
| RANTES | 0.75 |
| SCF | 0.80 |
| sTNF RII | 0.75 |
| TGF-β3 | 0.80 |
| TIMP-1 | 0.75 |
| TIMP-2 | 0.80 |
| TNF-β | 0.70 |
| TPO | 0.75 |

TABLE 2B

| | Relative Fold | | Absolute Fold | |
|---|---|---|---|---|
| Protein | Change (n = 51) | q-value | Change (n = 187) | p-value |
| MIP-1d | −0.54291 | 0.0165 | | |
| PDGF-BB | −0.53687 | 0.0165 | −0.135 | 0.891 |
| LEPTIN(OB) | −0.47625 | 0.0165 | −0.357 | 0.0018 |
| IL-6 R | −0.6763 | 0.0165 | | |
| BDNF | −0.53628 | 0.0165 | −0.355 | 0.0006 |
| TIMP-1 | −0.71622 | 0.0165 | | |
| RANTES | −0.68299 | 0.0165 | −0.184 | 0.0144 |
| EGF | −0.56182 | 0.0165 | | |
| TIMP-2 | −0.75011 | 0.0165 | | |
| NAP-2 | −0.67257 | 0.0165 | | |
| sTNF RII | −0.70029 | 0.0165 | | |
| TNF-b | −0.64998 | 0.0165 | | |
| TPO | −0.71405 | 0.0165 | | |
| FGF-6 | −0.66467 | 0.0165 | | |
| NT-3 | −0.69805 | 0.0165 | | |
| bFGF | −0.67351 | 0.0165 | | |
| IL-3 | −0.75802 | 0.0165 | | |
| SCF | −0.73041 | 0.0165 | | |
| TGF-b3 | −0.76912 | 0.0165 | | |
| MSP-a | −0.76466 | 0.0165 | | |

As will be apparent to those of skill in the art, when replicate measurements are taken for the biomarker(s) tested, the measured value that is compared with the reference value is a value that takes into account the replicate measurements. The replicate measurements may be taken into account by using either the mean or median of the measured values as the “measured value.”

### Screening Prospective Agents for AD Biomarker Modulation Activity

The invention also provides methods of screening for candidate agents for the treatment of AD by assaying prospective candidate agents for activity in modulating sets of AD biomarkers. The screening assay may be performed either in vitro and/or in vivo. Candidate agents identified in the screening methods described herein may be useful as therapeutic agents for the treatment of AD.

The screening methods of the invention utilize the sets of AD biomarkers described herein and sets of AD biomarker polynucleotides as “drug targets.” Prospective agents are tested for activity in modulating a drug target in an assay system. As will be understood by those of skill in the art, the mode of testing for modulation activity for each individual AD biomarker in the set will depend on the AD biomarker and the form of the drug target used (e.g., protein or gene). A wide variety of suitable assays are known in the art.

When the AD biomarker protein itself is the drug target, prospective agents are tested for activity in modulating levels or activity of the protein itself. Modulation of levels of an AD biomarker can be accomplished by, for example, increasing or reducing half-life of the biomarker protein. Modulation of activity of an AD biomarker can be accomplished by increasing or reducing the availability of the AD biomarker to bind to its cognate receptor(s) or ligand(s).

When an AD biomarker polynucleotide is the drug target, the prospective agent is tested for activity in modulating synthesis of the AD biomarker. The exact mode of testing for modulatory activity of a prospective agent will depend, of course, on the form of the AD biomarker polynucleotide selected for testing. For example, if the drug target is an AD biomarker polynucleotide, modulatory activity is typically tested by measuring either mRNA transcribed from the gene (transcriptional modulation) or by measuring protein produced as a consequence of such transcription (translational modulation). As will be understood by those in the art, many assay formats will utilize a modified form of the AD biomarker gene where a heterologous sequence (e.g., encoding an expression marker such as an enzyme or an expression tag such as oligo-histidine or a sequence derived from another protein, such as myc) is fused to (or even replaces) the sequence encoding the AD biomarker protein. Such heterologous sequence(s) allow for convenient detection of levels of protein transcribed from the drug target.

Prospective agents for use in the screening methods of the invention may be chemical compounds and/or complexes of any sort, including both organic and inorganic molecules (and complexes thereof). As will be understood in the art, organic molecules are most commonly screened for AD biomarker modulatory activity. In some situations, the prospective agents for testing will exclude the target AD biomarker proteins.

Screening assays may be in any format known in the art, including cell-free in vitro assays, cell culture assays, organ culture assays, and in vivo assays (i.e., assays utilizing animal models of AD). Accordingly, the invention provides a variety of embodiments for screening prospective agents to identify candidate agents for the treatment of AD.

In some embodiments, prospective agents are screened to identify candidate agents for the treatment of AD in a cell-free assay. Each prospective agent is incubated with the drug target in a cell-free environment, and modulation of the AD biomarker is measured. Cell-free environments useful in the screening methods of the invention include cell lysates (particularly useful when the drug target is an AD biomarker gene) and biological fluids such as whole blood or fractionated fluids derived therefrom such as plasma and serum (particularly useful when the AD biomarker protein is the drug target). When the drug target is an AD biomarker gene, the modulation measured may be modulation of transcription or translation. When the drug target is the AD biomarker protein, the modulation may of the half-life of the protein or of the availability of the AD biomarker protein to bind to its cognate receptor or ligand.

In other embodiments, prospective agents are screened to identify candidate agents for the treatment of AD in a cell-based assay. Each prospective agent is incubated with cultured cells, and modulation of target AD biomarker is measured. In certain embodiments, the cultured cells are astrocytes, neuronal cells (such as hippocampal neurons), fibroblasts, or glial cells. When the drug target is an AD biomarker gene, transcriptional or translational modulation may be measured. When the drug target is the AD biomarker protein, the AD biomarker protein is also added to the assay mixture, and modulation of the half-life of the protein or of the availability of the AD biomarker protein to bind to its cognate receptor or ligand is measured.

Further embodiments relate to screening prospective agents to identify candidate agents for the treatment of AD in organ culture-based assays. In this format, each prospective agent is incubated with either a whole organ or a portion of an organ (such as a portion of brain tissue, such as a brain slice) derived from a non-human animal and modulation of the target AD biomarker is measured. When the drug target is an AD biomarker gene, transcriptional or translational modulation may be measured. When the drug target is the AD biomarker protein, the AD biomarker protein is also added to the assay mixture, and modulation of the half-life of the protein or of the availability of the AD biomarker protein to bind to its cognate receptor is measured.

Additional embodiments relate to screening prospective agents to identify candidate agents for the treatment of AD utilizing in vivo assays. In this format, each prospective agent is administered to a non-human animal and modulation of the target AD biomarker is measured. Depending on the particular drug target and the aspect of AD treatment that is sought to be addressed, the animal used in such assays may either be a “normal” animal (e.g., C57 mouse) or an animal which is a model of AD. A number of animal models of AD are known in the art, including the 3×Tg-AD mouse (Caccamo et al., 2003, Neuron 39(3):409-21), mice over expressing human amyloid beta precursor protein (APP) and presenilin genes (Westaway et al., 1997, Nat. Med. 3(1):67-72), and others (see Higgins et al., 2003, Behay. Pharmacol. 14(5-6):419-38). When the drug target is an AD biomarker gene, transcriptional or translational modulation may be measured. When the drug target is the AD biomarker protein, modulation of the half-life of the target AD biomarker or of the availability of the AD biomarker protein to bind to its cognate receptor or ligand is measured.

The exact mode of measuring modulation of each target AD biomarker in the set will, of course, depend on the identity of the AD biomarker, the format of the assay, and the preference of the practitioner. A wide variety of methods are known in the art for measuring modulation of transcription, translation, protein half-life, protein availability, and other aspects which can be measured. In view of the common knowledge of these techniques, they need not be further described here.

### Kits

The invention provides kits for carrying out any of the methods described herein. Kits of the invention may comprise at least one reagent specific for each AD biomarker in the set, and may further include instructions for carrying out a method described herein. Kits may also comprise AD biomarker reference samples, that is, useful as reference values. Kits may comprise any set of biomarkers (and/or reagents specific for the set of biomarkers) as described herein. A set of AD diagnosis markers for use in kits provided herein comprises: MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, the set further comprises TRAIL R4 and IGFBP-6. In some embodiments, the set further comprises one or more additional biomarkers. In some embodiments, the kit comprises at least one reagent specific for each AD biomarker in the set, wherein the AD biomarkers comprise MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, the kit further comprises at least one reagent specific for: TRAIL R4 and IGFBP-6. In some embodiments, the kit further comprises at least one reagent specific for one or more additional biomarkers. The kits may be used in any of the methods as disclosed herein, including for example, methods to diagnose AD or to aid in the diagnosis of AD, or to diagnose AD as distinguished from other non-AD neurodegenerative diseases or disorders, such as for example PD and PN.

In additional examples, a kit comprises at least one AD diagnosis biomarker for use in normalizing data from experiments. In some examples, a kit comprises at least one of TGF-beta and TGF-beta 3 for use in normalizing data and in other examples, a kit comprises both TGF-beta and TGF-beta 3 for use in normalizing data. In some embodiments, the reagent(s) specific for an AD biomarker is an affinity reagent.

Kits comprising a single reagent specific for an AD biomarker will generally have the reagent enclosed in a container (e.g., a vial, ampoule, or other suitable storage container), although kits including the reagent bound to a substrate (e.g., an inner surface of an assay reaction vessel) are also contemplated. Likewise, kits including more than one reagent may also have the reagents in containers (separately or in a mixture) or may have the reagents bound to a substrate.

In some embodiments, the AD biomarker-specific reagent(s) will be labeled with a detectable marker (such as a fluorescent dye or a detectable enzyme), or be modified to facilitate detection (e.g., biotinylated to allow for detection with a avidin- or streptavidin-based detection system). In other embodiments, the AD biomarker-specific reagent will not be directly labeled or modified.

Certain kits of the invention will also include one or more agents for detection of bound AD biomarker-specific reagent. As will be apparent to those of skill in the art, the identity of the detection agents will depend on the type of AD biomarker-specific reagent(s) included in the kit, and the intended detection system. Detection agents include antibodies specific for the AD biomarker-specific reagent (e.g., secondary antibodies), primers for amplification of an AD biomarker-specific reagent that is nucleotide based (e.g., aptamer) or of a nucleotide ‘tag’ attached to the AD biomarker-specific reagent, avidin- or streptavidin-conjugates for detection of biotin-modified AD biomarker-specific reagent(s), and the like. Detection systems are well known in the art, and need not be further described here.

A modified substrate or other system for capture of AD biomarkers may also be included in the kits of the invention, particularly when the kit is designed for use in a sandwich-format assay. The capture system may be any capture system useful in an AD biomarker assay system, such as a multi-well plate coated with an AD biomarker-specific reagent, beads coated with an AD biomarker-specific reagent, and the like. Capture systems are well known in the art and need not be further described here.

In certain embodiments, kits for use in the methods disclosed herein include the reagents in the form of an array. The array includes at least two different reagents specific for AD biomarkers (each reagent specific for a different AD biomarker) bound to a substrate in a predetermined pattern (e.g., a grid). Accordingly, the present invention provides arrays comprising reagents for AD diagnosis markers including, but not limited to MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, the array further comprises reagents for TRAIL R4 and IGFBP-6. In some embodiments, the array further comprises reagents for one or more additional biomarkers. The present invention provides arrays comprising AD diagnosis markers including, but not limited to MCSF, RANTES, GCSF, PARC, ANG-2, IL-11, EGF, MCP-3, IL-3, MIP-1delta, ICAM-1, PDGF-BB, IL-8, GDNF, IL-1a, and TNF-a. In some embodiments, the array further comprises TRAIL R4 and IGFBP-6. In some embodiments, the array further comprises one or more additional biomarkers. The localization of the different AD biomarker-specific reagents (the “capture reagents”) allows measurement of levels of a number of different AD biomarkers in the same reaction. Kits including the reagents in array form are commonly in a sandwich format, so such kits may also comprise detection reagents. Normally, the kit will include different detection reagents, each detection reagent specific to a different AD biomarker. The detection reagents in such embodiments are normally reagents specific for the same AD biomarkers as the reagents bound to the substrate (although the detection reagents typically bind to a different portion or site on the AD biomarker target than the substrate-bound reagents), and are generally affinity-type detection reagents. As with detection reagents for any other format assay, the detection reagents may be modified with a detectable moiety, modified to allow binding of a separate detectable moiety, or be unmodified. Array-type kits including detection reagents that are either unmodified or modified to allow binding of a separate detectable moiety may also contain additional detectable moieties (e.g., detectable moieties which bind to the detection reagent, such as labeled antibodies which bind unmodified detection reagents or streptavidin modified with a detectable moiety for detecting biotin-modified detection reagents).

The instructions relating to the use of the kit for carrying out the invention generally describe how the contents of the kit are used to carry out the methods of the invention. Instructions may include information as sample requirements (e.g., form, pre-assay processing, and size), steps necessary to measure the AD biomarker(s), and interpretation of results.

Instructions supplied in the kits of the invention are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured values obtained using the reagents included in the kit.

The following Examples are provided to illustrate the invention, but are not intended to limit the scope of the invention in any way.

### EXAMPLES

### Example 1

The following Example was published in Nature Medicine 13, 1359-1362 (2007), with an online publication date of Oct. 14, 2007, and which is herein incorporated by reference in its entirety.

Molecular classification and class prediction of Alzheimer's disease based on secreted signaling proteins in plasma

### ABSTRACT

Alzheimer's disease (AD) is a fatal dementia affecting one in eight people at age 65. Early diagnosis is urgently needed to effectively treat patients and to develop new therapies. Using antibody-based filter arrays and a shrunken centroid-based algorithm we demonstrate that relative concentrations of 18 signaling proteins in plasma allow for classification of blinded samples from AD patients and controls with 90% sensitivity and 88% specificity. More importantly, the same proteins and algorithm also classified as AD blinded samples from patients with mild cognitive impairment (MCI) who progressed to AD 2-6 years later (91% sensitivity) against samples from patients who developed other dementias or remained MCI (72% specificity). Biological analysis of the 18 markers suggests for the first time a systemic dysregulation of hematopoiesis, immune responses, and apoptosis in pre-symptomatic AD. These findings indicate that our set of plasma signaling proteins can serve as a phenotypic biomarker for AD and may help in the understanding of the underlying disease process.

### Introduction

Alzheimer's disease (AD) results in a progressive loss of cognitive function and dementia in all but a few familial forms of the disease but its cause remains unknown¹. The diagnosis is largely based on a complex set of clinical examination parameters to exclude other forms of dementia and in expert research centers in the US the diagnostic accuracy reaches 80% sensitivity and 70% specificity². As of today, there are no simple molecular tests available to aid classification of dementias. Even more challenging, and restricted to highly specialized clinics, is the diagnosis of patients with mild cognitive impairment (MCI), a condition with greatly increased risk to develop AD³. As a result of the diagnostic difficulties it is estimated that the disease process may have started many years before the typical patient is diagnosed with AD and an estimated quarter million of AD patients per year are not diagnosed at all in the US⁴.

While current AD medications are not disease modifying they can improve activities of daily living and delay the median time of nursing home placement by about 17-21 months⁵. It is generally agreed upon that earlier diagnosis would increase the chances of a favorable response to available drugs and, more importantly, it would help in the development and testing of new treatments. Promising new diagnostic markers of AD include various imaging techniques for structural or molecular changes associated with the disease⁶ but these methods are expensive and currently limited to academic centers. In addition, the molecular markers β-amyloid and hyperphosphorylated tau protein, which accumulate in the brain of AD patients, have been demonstrated to correlate and predict AD when measured in cerebrospinal fluid (CSF) of MCI patients¹. However, CSF collection is invasive and routine testing would be difficult in large numbers of at-risk patients.

The utility of a plasma biomarker for the classification of AD, and in particular for identifying pre-symptomatic patients with MCI that will convert to AD, would be an improvement over existing measures for the clinical assessment of dementia. Blood-derived proteomic marker assays are used for diagnosis and monitoring of disease processes in various tissues⁷. This may be possible because the blood serves as a complex carrier for signaling proteins, hormones and other transmitters that are secreted by affected tissues and by blood-derived cells that interact with these tissues. Since the brain controls many body functions via the release of signaling proteins and because central and peripheral immune and inflammatory mechanisms are increasingly implicated in brain diseases, we hypothesized that the disease process of AD would lead to characteristic changes in concentrations of signaling proteins in the blood, generating a detectable disease-specific molecular phenotype.

### Results

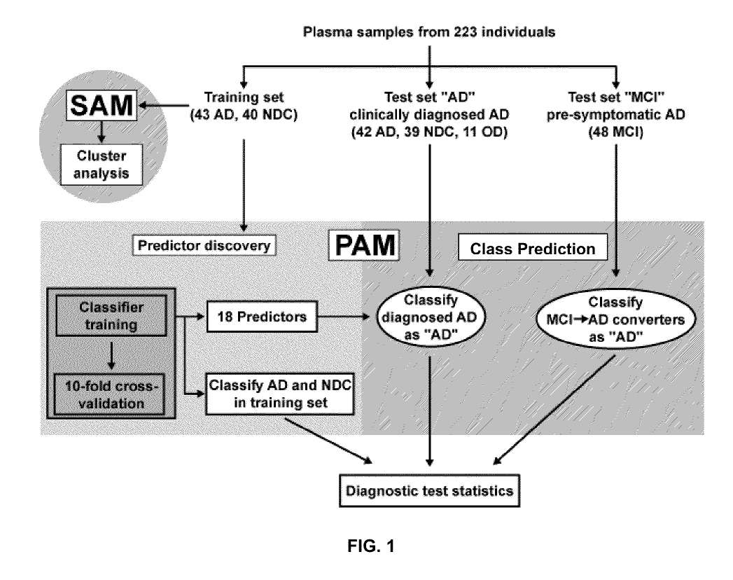

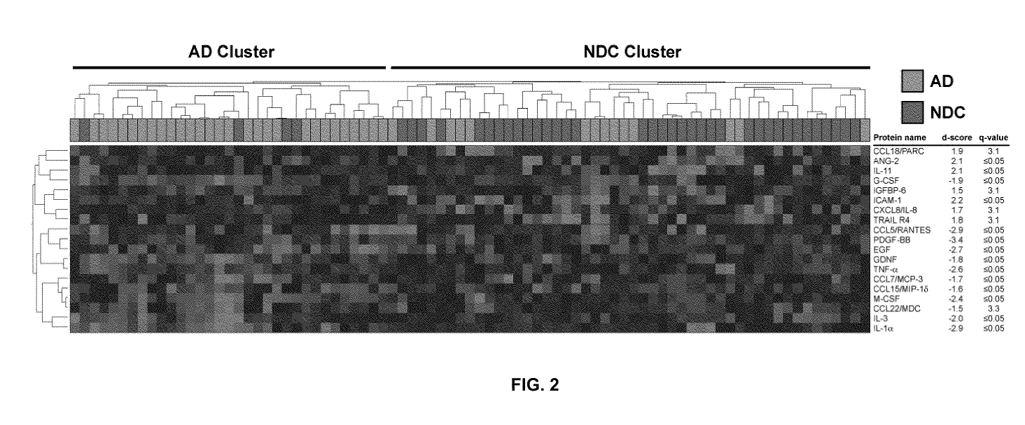

Identification of distinct expression patterns of secreted signaling proteins in AD and NDC in plasma. To identify potential differences in plasma concentration of cellular signaling proteins associated with AD, we measured 120 known cytokines, chemokines, growth factors and related proteins (Table 3) using filter based, arrayed sandwich ELISAs⁸. We collected 223 archived EDTA plasma samples from patients with pre-symptomatic to late stage AD and various controls (Table 4). These samples were collected from seven different AD research centers and clinics to avoid the potential identification of patterns associated with a particular AD center rather than with AD. From a total of 85 AD patients and 79 non-demented controls (NDC) we generated two matched sets of samples with respect to diagnosis, age, sex, and source (FIG. 1; Table 4). One set served as training set for supervised classification of AD and NDC whereas the other sample set was used to test the algorithm for class prediction of blinded samples (see below). Initial statistical analysis of the training set by Significance Analysis of Microarray (SAM;⁹) identified 19 proteins with highly significant differences in expression (q-value <3.4%) between the two groups (FIG. 2). Unsupervised clustering¹⁰ of all 83 samples in the training set with these 19 markers produced two main sample clusters, an “AD cluster” containing mostly AD samples and a “NDC cluster” that contained a majority of the NDC samples (FIG. 2). FIG. 2 shows a cluster diagram illustrating a perceivable difference in expression patterns between 43 Alzheimer's patient blood samples and 40 non-demented control patient blood samples for 19 plasma biomarkers. The 19 plasma biomarkers, from top to bottom in the order listed in FIG. 2 are: CCL18/PARC, ANG-2, IL-11, G-CSF, IGFBP-6, ICAM-1, CXCL8/IL-8, TRAIL R4, CCL5/RANTES, PDGF-BB, EGF, GDNF, TNF-α, CCL7/MCP-3, CCL15/MIP-1δ, MCSF, CCL22/MDC, IL-3, IL-1α. These results demonstrate that concentrations of many secreted signaling proteins in plasma differ considerably between AD and NDC and that a distinct protein expression pattern is associated with AD and NDC, respectively.

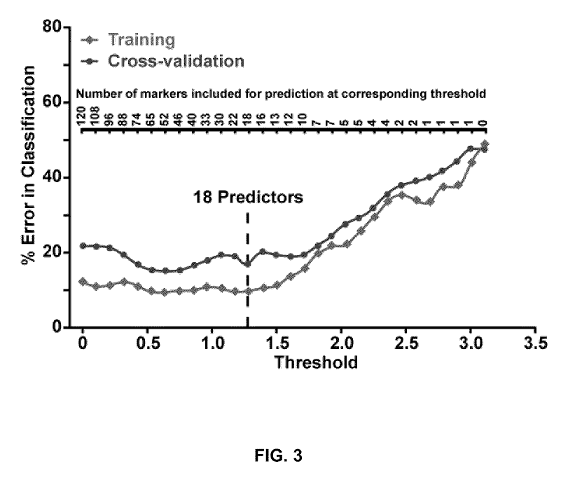

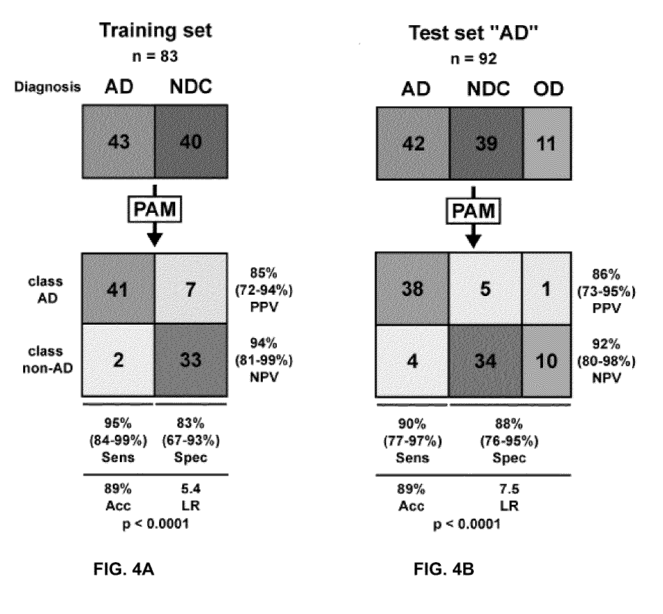

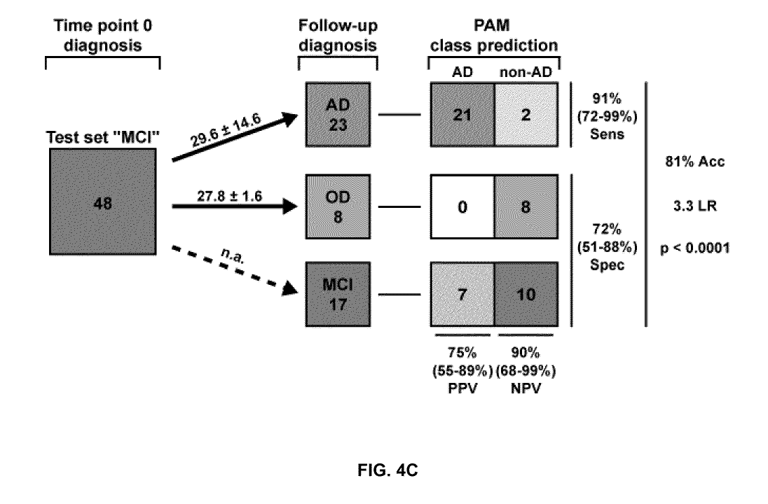

Class prediction of clinically diagnosed AD based on relative concentrations of signaling proteins in plasma. To find an AD-specific and predictive plasma signaling protein signature that could serve as a potential biomarker phenotype, we applied to the above training set a shrunken centroid algorithm packaged in the statistical tool Predictive Analysis of Microarray (PAM;¹¹). An internal cross-validation method is provided to assess and minimize classification errors and to avoid overfitting (FIG. 1). PAM identified 18 predictors (FIG. 3, for list see Table 3) in the training set (43 AD; 40 NDC) and classified AD and NDC samples with 89% accuracy and a likelihood ratio of 5.4 (p<0.0001; FIG. 4a). This ratio indicates in our study how many times more likely a patient with AD is classified correctly by the test compared to a subject without AD.

To assess the performance of PAM and the 18 predictors in classification we carried out a two-class prediction in a blinded test set containing samples collected from 42 AD patients, 39 NDC, and 11 other dementia (OD) patients (FIG. 1). Notably, the sample donors for AD and NDC in this test set were of similar age and sex in comparison to the donors for the training set, and the cognitive states as measured by MMSE demonstrated a similar distribution (Table 4). None of the samples in this test set were used in the previous training procedure. PAM classified samples in the test set with 91% sensitivity, 87% specificity (89% accuracy) and a likelihood ratio of 7.5 (p<0.0001; FIG. 4b). Moreover, PAM classified 10 out of 11 OD samples as “non-AD” (91% specificity).

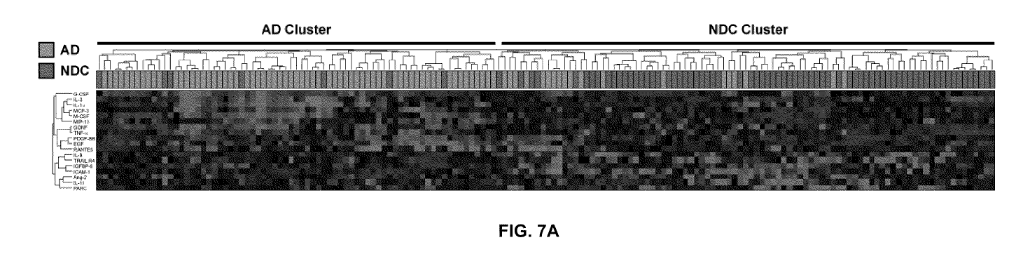

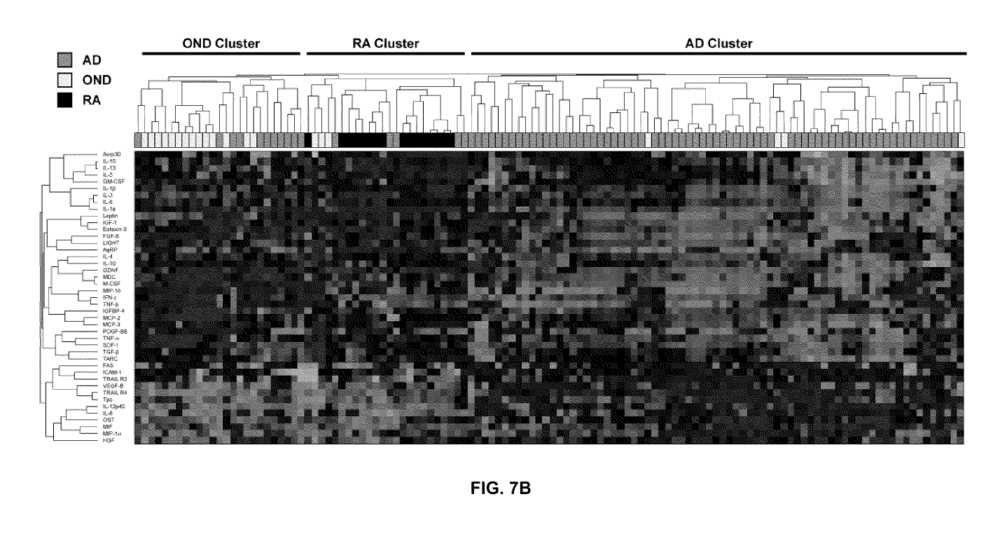

For 9 of the 42 AD patients in the test set the diagnosis of AD was confirmed by postmortem autopsy and PAM correctly classified 8 of them (89% sensitivity). Interestingly, segmentation analysis of the combined training and test sets (85 AD, 79 NDC) by unsupervised clustering based on correlation of relative concentrations of the 18 markers also produced two major segments consisting of mostly AD samples or NDC in the two groups, respectively (FIG. 7A). FIG. 7A shows a cluster diagram illustrating a perceivable difference in expression patterns between 85 Alzheimer's patient blood samples and 79 non-demented control patient blood samples for 18 plasma biomarkers. The 18 plasma biomarkers, from top to bottom in the order listed in FIG. 7A are:

G-CSF, IL-3, IL-1α, MCP-3, M-CSF, MIP-18, GDNF, TNF-α, PDGF-BB, EGF, RANTES, IL-8, TRAIL R4, IGFBP-6, ICAM-1, ANG-2, IL-11, PARC. Together, these results demonstrate that differences in expression levels of a small number of secreted signaling proteins provide an AD plasma biomarker phenotype that enables the classification of AD patients and NDC subjects with high accuracy.

Class prediction of pre-symptomatic AD in MCI patients. A substantial therapeutic and health economic benefit could be realized from detection of AD biomarker phenotypes among MCI patients who later develop AD. To determine whether this is possible, we analyzed plasma samples from two previously published cohorts of MCI patients that were followed longitudinally and converted to AD, developed other dementias, or remained MCI (Table 4)^{12,13}. The plasma samples were collected at the initial diagnosis of MCI (time point 0) and patients obtained a final follow-up diagnosis for this study after 2-6 years. We again used the 18 predictors that demonstrated best discriminatory power to classify clinically diagnosed AD and NDC, and applied the PAM two-class prediction algorithm to the test set “MCI” (FIG. 1). PAM classified 21 of 23 MCI patients who developed AD 2-5 years later as “AD” (sensitivity 91%; FIG. 4c). All eight MCI patients who later developed other dementias were correctly classified as “non-AD”, indicating that these MCI→OD converters can be discriminated from MCI→AD converters with high specificity (FIG. 4c). Out of 17 MCI patients who were still diagnosed as MCI 4-6 years after blood draw, 7 were classified as “AD” and it will be interesting to know whether some of them will still develop AD. On the other hand, the 10 MCI patients who were classified as “non-AD” may develop other dementias or represent a different form of the MCI syndrome that does not lead to AD¹⁴. Our data indicate that a highly specific plasma biomarker phenotype exists for AD very early in the disease process, years before a clinical diagnosis of AD can be made.

A distinct molecular pattern of plasma signaling proteins discriminate AD from other neurological and inflammatory diseases. One could argue that the AD-specific plasma protein biomarker phenotype we identified is simply associated with inflammatory processes, which commonly occur during chronic degenerative diseases. To address this possibility, we analyzed concentrations of plasma proteins in 21 samples from a cohort of patients with other neurological diseases (OND) and a cohort of 16 patients with rheumatoid arthritis (RA; Table 4), and compared these cohorts with all 85 AD samples used in the training set and the “AD” test set. Except for Parkinson's disease, the OND have a variable inflammatory component, and RA is a prototypical chronic inflammatory condition. Out of 120 proteins measured in plasma, SAM identified 43 to have significantly different levels between AD samples and other diseases (q-value ≦0.05%; for list of markers see Table 4). Unsupervised clustering using the 43 proteins generated three main clusters containing mostly AD, OND, or RA samples, respectively (FIG. 7B). FIG. 7B shows a cluster diagram illustrating a perceivable difference in expression patterns between 85 Alzheimer's patient blood samples, 21 blood samples from 21 patients with another form of dementia (non-Alzheimer's), and 16 blood samples from 16 patients with rheumatoid arthritis, for 43 plasma biomarkers.

The 43 plasma biomarkers, from top to bottom in the order listed in FIG. 7B are: Acrp30 (Adiponectin), IL-15, IL-13, IL-5, GM-CSF, IL-1β, IL-3, IL-6, IL-1α, Leptin, IGF-1, Eotaxin-3, FGF-6, LIGHT, AgRP, IL-4, IL-10, GDNF, MDC, M-CSF, MIP-1δ, IFN-γ, TNF-β, IGFBP-4, MCP-2, MCP-3, PDGF-BB, TNF-α, SDF-1, TGF-13, TARC, FAS, ICAM-1, TRAIL R3, VEGF-B, TRAIL R4, Tpo, IL-12p40, IL-8, OST, MIF, MIP-1α, HGF. Although the numbers of samples in the OND- or RA-cohorts were low, our results reveal disease-specific differences in signaling protein concentrations and demonstrate a distinct molecular pattern different from AD that allows for differential clustering. This is in line with recent observations obtained in RA with other peripheral immune markers measured on a different platform¹⁵.

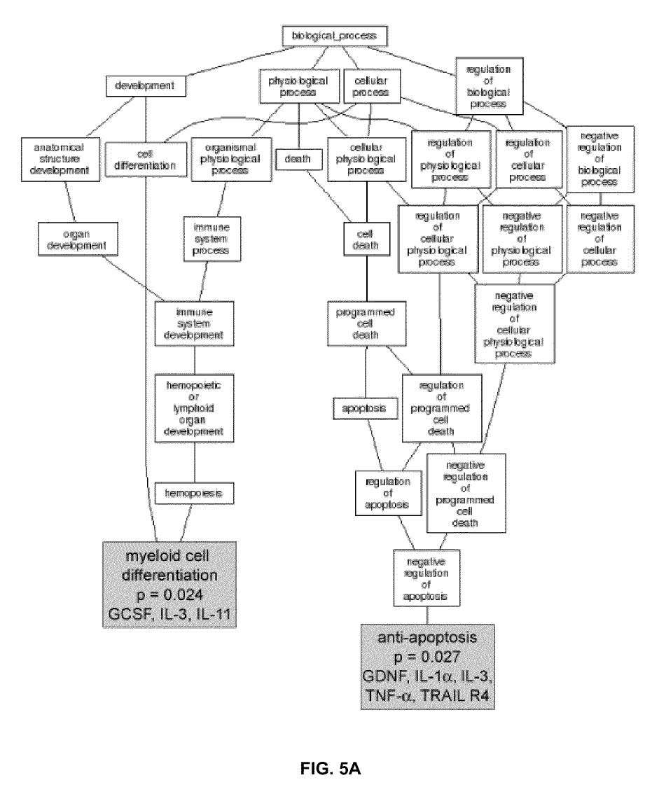

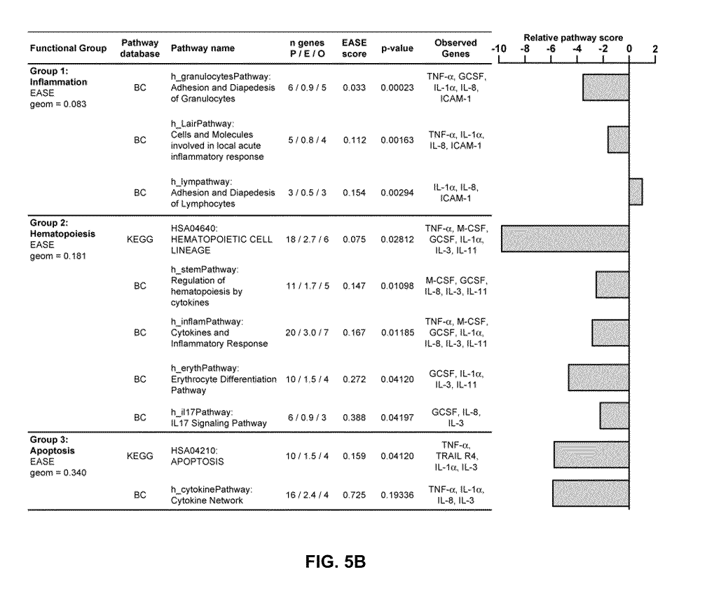

AD molecular biomarker phenotype of 18 predictor proteins points to dysregulation of peripheral and central biological pathways and processes. To understand the potential biological meaning of the 18 markers that characterize AD we used functional annotation tools and searched PubMed for information relevant to the 18 predictors and AD. The online gene set analysis toolkit WebGestalt found seven of the 18 markers to be significantly overrepresented in the human gene ontology sub-categories anti-apoptosis (p=0.027) and myeloid cell differentiation (p=0.024) (FIG. 5a). To understand which metabolic and regulatory pathways are involved in these processes we searched with WebGestalt and the Database for Annotation, Visualization, and Integrated Discovery (DAVID) 2006 for protein overrepresentations in Kyoto Encyclopedia of Genes and Genomes (KEGG) and BioCarta pathways. Both online tools identified the same ten biological pathways with high significance (FIG. 5b) and DAVID clustered them into the three functional groups “inflammation”, “hematopoiesis”, and “apoptosis”. The overall effect of up- or down-regulation of the observed predictors on these pathways was examined by calculating a relative score for each pathway, which was obtained by adding up the positive and negative SAM d-scores (FIG. 2). The overrepresented proteins predict a negative impact on the majority of the pathways.

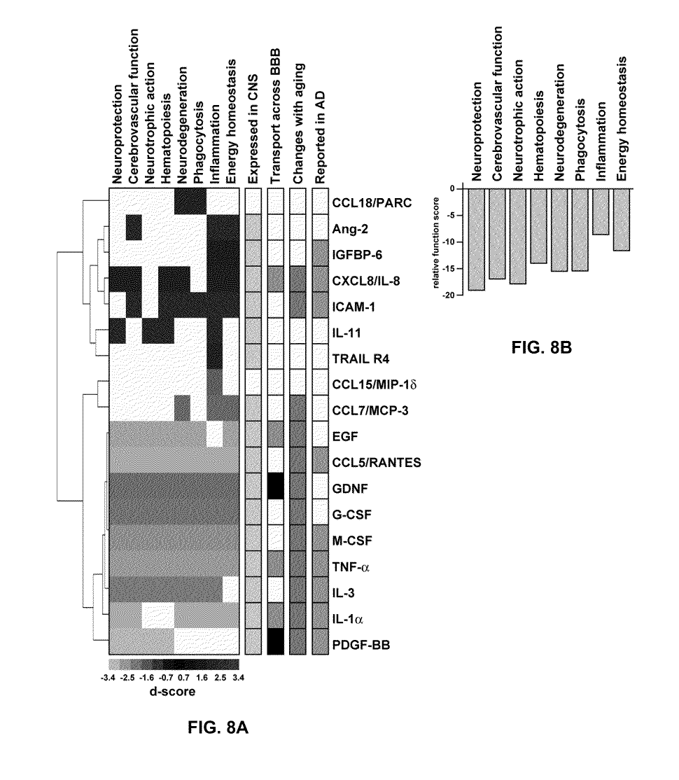

Because the above online tools do not provide sufficient information on brain- and AD-related functions, we searched PubMed directly for reports linking the 18 predictor proteins with AD, neurodegeneration, and other potentially relevant biological processes or functions. This analysis confirmed an overall reduction in factors associated with hematopoiesis and inflammation and also pointed to deficits in neuroprotection, neurotrophic activity, phagocytosis, and energy homeostasis (indicated by relative function scores, see FIGS. 8A and 8B). We also found that except for CCL15/MIP-1δ and CCL18/PARC all proteins have been reported to be produced in the central nervous system (CNS) apart from a number of other tissues and a few are transported across the blood brain barrier in a saturable way.

No reports on AD-related expression changes in the periphery or in the CNS could be found for nine of these proteins (FIGS. 8A and 8B). Consistent with the changes of the relative plasma concentrations we observed in AD compared with NDC, intercellular adhesion molecule (ICAM)-1, CXCL8/IL-8, and insulin-like growth factor binding protein (IGFBP)-6 have previously been found at higher levels in AD serum, CSF, or brain (Table 5). The multifunctional cytokine tumor necrosis factor (TNF)-α, which has neuroprotective as well as neurotoxic effects¹⁶, has previously been reported to be reduced in AD brains and serum in some studies although others could not confirm this (Table 5).

Together, our functional analysis of the 18 signaling proteins constituting the AD molecular biomarker phenotype is the first demonstration of a dysregulation of hematopoiesis, immune responses, apoptosis, and neuronal support already in pre-symptomatic AD patients.

### Discussion

Our study demonstrates that peripheral changes in signaling proteins are associated with AD and can be used to classify the disease. Moreover, our findings indicate that hematopoiesis, immune and other biological processes are altered in the disease early on, several years before a clinical diagnosis of AD is made. The changes are specific compared with a number of related dementias, other neurological diseases, or arthritis, suggesting a crosstalk between disease-specific lesions in the CNS and the periphery that might be more important than previously appreciated.

It seems reasonable that CSF is a potential source of protein information about a disease process and it has indeed been demonstrated recently that CSF levels of β-amyloid and hyperphosphorylated tau can predict conversion from MCI to AD¹². In fact some of the samples in our MCI conversion experiment were used in the current study (FIG. 4c). Individual plasma proteins on the other hand have not been found to predictably discriminate AD from healthy controls or other dementias and they have failed to predict progression to AD¹⁷. In contrast, in other disease areas plasma proteins, singly or in patterns, were able to discriminate disease from control, identify disease stages or subgroups, or allow for the prediction of disease progression¹⁸⁻²¹.

In order to find predictive plasma markers for AD we reasoned that secreted signaling proteins, which may have the highest information content of all proteins, would be the most likely to differ between disease and control—even if the disease affects the brain. Since the brain communicates with most tissues through the blood and blood-borne immune cells patrol the brain it is conceivable that the network of communication, and thus the levels of specific signaling proteins in the blood may be dysregulated in diseases of the brain. This is supported by studies of gene expression patterns in blood cells, which were sufficient to predict early Parkinson's disease²², and possibly AD as well^{23,24}. Other studies reported changes in leukocyte subset distribution in blood or cytokine secretion from blood cells in MCI²⁵ or AD^{26,27}. Whether the signaling proteins we identified are CNS derived or a peripheral response to CNS damage and whether they are cause or consequence of the disease is unknown at this point. Based on our findings, however, they appear to be characteristically changed in the blood already several years before AD is clinically diagnosed, making it very unlikely that the 18 proteins we discovered are simply a result of full-blown neurodegeneration or agonal state of AD. Rather, some of these proteins may indicate a previously unrecognized peripheral or central dysfunction early in the disease process.

The potential role of the immune system and blood-derived cells in neurodegeneration has received particular attention recently. Such cells enter the brain in AD^{28,29} or in mouse models of the disease³⁰ at increased frequencies. Limiting migration of monocyte/macrophages to sites of A13 accumulation leads to a prominent worsening of disease in AD mouse models^{31,32}. It is of particular interest therefore, that we observed in AD plasma reduced levels of CCL7/monocyte chemotactic protein (MCP)-3, which is a ligand for CCR2 and one of our predictive markers (FIGS. 8A and 8B). Moreover, several unbiased, automated functional annotation tools identified hematopoiesis and inflammation pathways to be affected and possibly reduced in AD. This is in line with observations in isolated immune cells^{24,26,33} and reports, which indicate that lack of monocyte-colony stimulating factor (M-CSF) receptors in rodents can lead to Aβ accumulation in the brain³⁴. We also found plasma markers associated with apoptosis to be reduced (FIGS. 5A and 5B) and it will be interesting to study some of the proteins implicated in this process more closely in AD.

Our observation that changes in plasma proteins in a major neurodegenerative disease are indicative of its earliest known stage implies that similar signatures may exist for other CNS diseases and that such changes may hold potential clues for both treatment and diagnosis. The current study provides proof of concept that molecular patterns of signaling proteins in plasma can represent a disease-specific biomarker phenotype that may help in the differential diagnosis of AD and the identification of pre-symptomatic patients several years before progression to clinically detectable AD.

### Methods

### Plasma Samples.

A total of 260 archived EDTA plasma samples were obtained from academic centers specialized on neurological or neurodegenerative diseases (Table 3). Plasma was produced by standard EDTA-blood processing, then frozen and stored in aliquots at ±80° C. Patient consent was obtained at the local institutions.

Antibody Arrays.

A total of 120 proteins were measured with cytokine antibody arrays (Raybiotech, Norcross, Ga.) according to the manufacturer's instructions⁸. Briefly, for each plasma sample two nitrocellulose membranes, each containing 60 different antibodies in duplicate spots were blocked, incubated with plasma, washed, and then incubated with a cocktail of biotin-conjugated antibodies specific for the different proteins. Membranes were developed with streptavidin-conjugated peroxidase and ECL chemiluminescense reagent and exposed to autoradiographic film (BioMax Lite, Kodak).

### Data Extraction and Transformation.

Autoradiographic films were scanned and digitized spots were quantified with the Imagene 6.0 data extraction software (BioDiscovery Inc., El Segundo, Calif.). Local background intensities were subtracted from each spot, and the average of the duplicate spots for each protein was normalized to the average of 6 positive controls on each membrane. Repeat measurements of the same samples produced Pearson correlation coefficients R²>0.95 for the 120 protein measurements (n=4 samples; data not shown). For statistical analysis expression data from the two filters per sample were normalized to the median expression of all 120 proteins and then scaled to correct for large differences between expression levels on a sample-by-sample basis. For this the sample mean was adjusted to 0 and the standard deviation to 1 (Z score transformation;³⁵).

### Cytokine Antibody Array Data Analysis.

Significance analysis of microarray (SAM). Group differences between AD and NDC training set samples or AD and OND/RA samples were compared applying the SAM 3.00 algorithm (⁹; http://www-stat.stanford.edu/—tibs/SAM/index.html). SAM is a statistical tool that assigns a score (d-score) to each gene or protein on the basis of expression change relative to the standard deviation of repeated measurements. It then uses permutations of the repeated measurements to estimate the percentage of genes or proteins identified by chance, the false discovery rate, for which the significance is indicated by the q-value³⁶.

Unsupervised Clustering. A 2-way unsupervised clustering algorithm with a top down repeated bisection approach was used in the clustering package CLUTO 2.1.1 (³⁷; http://glaros.dtc.umn.edu/gkhome/cluto/cluto/download) to group proteins on the basis of similarity in pattern of expression over all samples.

Predictive analysis of microarray (PAM). A semi-supervised prediction analysis was performed using the statistical package PAM 2.3.1 with the statistical tool R (^{11,38}; http://www-stat.stanford.edu/˜tibs/PAM/index.html; FIG. 1). PAM performs a sample classification training routine from expression data via the nearest shrunken centroid procedure to find markers that discriminate best between two or more classes. After that it applies an internal cross-validation by 10-times randomly selecting 90% of the training samples in a class-balanced way to then predict each time the class labels on the remaining 10% of samples. This assesses and minimizes classification errors and avoids overfitting. The obtained minimal number of predictors is then used for a heterogeneity analysis to perform class prediction in a test set between a diseased group and normal control group. PAM demonstrated highest accuracy when compared with other algorithms on several public datasets (http://www-stat.stanford.edu/˜tibs/PAM/comparison.html;³⁹).

### Functional Profiling.

Gene ontology (GO) analysis. For a functional profiling of the 18 predictors the online gene set analysis toolkit WebGestalt (http://bioinfo.vanderbilt.edu/webgestalt;⁴⁰) was set to level 4 and p-value p≦0.05 to stratify the search for gene enrichment in human GO (www.geneontology.org) terms in comparison to our own reference list of 120 proteins measured by filter array. Significant gene overrepresentations found by a hypergeometric statistical test were illustrated as an enriched Directed Acyclic Graph (DAG).

Biological pathway analysis. To obtain an overview of metabolic and regulatory pathways affected by AD, WebGestalt was queried for enriched pathways in the open source databases Kyoto Encyclopedia of Genes and Genomes (KEGG, www.genome.ad.jp/kegg/) and BioCarta (http://www.biocarta.com/genes/index.asp). From our filter array 103 markers including 15 predictors were present in the KEGG pathways and 56 markers including 12 predictors in the BioCarta pathways, respectively. We identified overrepresented pathways by stratifying for at least three significantly (p≧0.05) enriched markers in each pathway. We verified the WebGestalt findings in the Database for Annotation, Visualization, and Integrated Discovery (DAVID) 2006, an online graph theory evidence-based method to agglomerate heterogeneous and widely distributed public databases (http://david.abcc.ncifcrf.gov/home.jsp;⁴¹). DAVID allows to search, rank and cluster functional gene or protein similarities within a set of genes or proteins of interest in order to unravel new biological processes associated with their cellular functions and pathways. The DAVID incorporated online module Expression Analysis Systematic Explorer (EASE) searched for overrepresented markers within the present proteins of interest in comparison to our protein reference list. EASE generated a gene representation score (EASE score; p-value of the Jackknife Fisher exact test), which DAVID used to arrange the enriched proteins and their corresponding pathways at lowest similarity and clustering stringency into functional groups. Because DAVID clustered the biological pathways by similarity into groups without providing a group name, we identified the three obtained groups based on the listed functions in a group. To better examine the overall effect of up- or down-regulation of the enriched predictors on the individual pathways, we calculated a relative pathway score by adding up the respective SAM-derived (FIG. 2) positive and negative d-scores of the individual markers in each pathway.

Brain- and AD-specific functions and findings. Because WebGestalt and DAVID do not allow for a precise search of brain- and AD-specific functions of the 18 predictors and to further confirm the findings of the two online tools used above, we performed our own investigation on PubMed (www.pubmed.gov) with the following keywords: neuroprotection, neurotrophic, neurodegeneration, cerebrovasculature, inflammation, phagocytosis, hematopoiesis, or energy metabolism. If at least one PubMed entry was found linking a given factor with the specified function in vivo, the corresponding SAM d-score was assigned to that keyword. If no entry was found, null was given. To order the markers and their keywords a hierarchical cluster algorithm with a pairwise similarity function was applied (Open Source Clustering Software Cluster 3.0, http://bonsai.ims.u-tokyo.ac.jp/˜mdehoon/software/clusted;^{10,42}). Cluster results were displayed using Java TreeView (⁴³; http://jtreeview.sourceforge.net/). For each keyword a qualitative functional score was calculated as described for the relative pathway score. Similarly, we searched for PubMed entries that describe expression of the 18 in the CNS, their ability to cross the blood-brain-barrier (BBB), or if they have been studied with regard to aging. In addition, we searched for reports in human AD or AD mouse models changes of expression levels (RNA and protein) or abnormal presence of the 18 predictive markers in plasma, serum, CSF, or brain parenchyma.

Pathways Analysis. Ingenuity Pathways Knowledge Base was used to build two independent networks based on 13 and 5 signaling proteins out of the set of 18 predictors, respectively. The 13-protein network received a high score by Ingenuity Pathways Analysis (IPA) and is primarily centered around TNF-α and M-CSF. Associated functions are cell-to-cell signaling and interaction, cellular growth, and proliferation (connective tissue, hematological, immune, and lymphoid), immune response, and cell death. The 5-protein network received a low IPA score and is primarily centered on EGF. Associated functions are gene expression, cancer, and cellular movement. In both networks most interactions are based on indirect activation of groups or complexes of kinases. Decreased concentrations of many of the predictive markers connected in the two networks may lead to an overall reduced activation of these kinases.

### Diagnostic Test Statistics.

Standard diagnostic test statistics were calculated in a 2×2 contingency table with 95% confidence intervals and two-sided p-value of the Fisher's exact test using InStat 3.0 (GraphPad Software Inc., San Diego, Calif.).

### REFERENCES

1. Blennow, K., de Leon, M. J. & Zetterberg, H. Alzheimer's disease. Lancet 368, 387-403 (2006).
2. Knopman, D.S., et al. Practice parameter: diagnosis of dementia (an evidence-based review). Report of the Quality Standards Subcommittee of the American Academy of Neurology. Neurology 56, 1143-1153 (2001).
3. Storandt, M., Grant, E. A., Miller, J. P. & Morris, J. C. Rates of progression in mild cognitive impairment and early Alzheimer's disease. Neurology 59, 1034-1041 (2002).
4. Alzheimer's Association National Office. Early onset dementia: a national challenge, a future crisis. (Alzheimer's Association National Office, Ill., 2006).
5. Geldmacher, D. S., Provenzano, G., McRae, T., Mastey, V. & Ieni, J. R. Donepezil is associated with delayed nursing home placement in patients with Alzheimer's disease. J Am Geriatr Soc 51, 937-944 (2003).
6. Buckner, R. L. Memory and executive function in aging and AD: multiple factors that cause decline and reserve factors that compensate. Neuron 44, 195-208 (2004).
7. Anderson, L. Candidate-based proteomics in the search for biomarkers of cardiovascular disease. J Physiol 563, 23-60 (2005).
8. Huang, R. P. Cytokine protein arrays. Methods Mol Biol 264, 215-231 (2004).
9. Tusher, V. G., Tibshirani, R. & Chu, G. Significance analysis of microarrays applied to the ionizing radiation response. Proc Natl Acad Sci USA 98, 5116-5121 (2001).
10. Eisen, M. B., Spellman, P. T., Brown, P. O. & Botstein, D. Cluster analysis and display of genome-wide expression patterns. Proc Natl Acad Sci USA 95, 14863-14868 (1998).
11. Tibshirani, R., Hastie, T., Narasimhan, B. & Chu, G. Diagnosis of multiple cancer types by shrunken centroids of gene expression. Proc Natl Acad Sci USA 99, 6567-6572 (2002).
12. Hansson, O., et al. Association between CSF biomarkers and incipient Alzheimer's disease in patients with mild cognitive impairment: a follow-up study. Lancet Neurol 5, 228-234 (2006).
13. Assini, A., et al. Plasma levels of amyloid beta-protein 42 are increased in women with mild cognitive impairment. Neurology 63, 828-831 (2004).
14. Gauthier, S., et al. Mild cognitive impairment. Lancet 367, 1262-1270 (2006).
15. Hueber, W., et al. Proteomic analysis of secreted proteins in early rheumatoid arthritis: Anti-citrulline reactivity is associated with upregulation of proinflammatory cytokines. Ann Rheum Dis (2006).
16. Akiyama, H., et al. Inflammation and Alzheimer's disease. Neuroinflammation Working Group. Neurobiol. Aging 21, 383-421 (2000).
17. Teunissen, C. E., et al. Combination of serum markers related to several mechanisms in Alzheimer's disease. Neurobiol Aging 24, 893-902 (2003).
18. Petricoin, E. F., et al. Use of proteomic patterns in serum to identify ovarian cancer. Lancet 359, 572-577 (2002).
19. Mor, G., et al. Serum protein markers for early detection of ovarian cancer. Proc Natl Acad Sci USA 102, 7677-7682 (2005).
20. Sheta, E. A., Appel, S. H. & Goldknopf, I. L. 2D gel blood serum biomarkers reveal differential clinical proteomics of the neurodegenerative diseases. Expert Rev Proteomics 3, 45-62 (2006).
21. Kingsmore, S.F. Multiplexed protein measurement: technologies and applications of protein and antibody arrays. Nature reviews 5, 310-320 (2006).
22. Scherzer, C. R., et al. Molecular markers of early Parkinson's disease based on gene expression in blood. Proc Natl Acad Sci USA 104, 955-960 (2007).
23. Maes, O. C., et al. Transcriptional profiling of Alzheimer blood mononuclear cells by microarray. Neurobiol Aging (2006).
24. Fiala, M., et al. Ineffective phagocytosis of amyloid-beta by macrophages of Alzheimer's disease patients. J Alzheimers Dis 7, 221-232; discussion 255-262 (2005).
25. Magaki, S., Mueller, C., Dickson, C. & Kirsch, W. Increased production of inflammatory cytokines in mild cognitive impairment. Exp Gerontol (2006).
26. Richartz-Salzburger, E., et al. Altered lymphocyte distribution in Alzheimer's disease. J Psychiatr Res (2006).
27. Speciale, L., et al. Lymphocyte subset patterns and cytokine production in Alzheimer's disease patients. Neurobiol Aging (2006).
28. Togo, T., et al. Occurrence of T cells in the brain of Alzheimer's disease and other neurological diseases. J Neuroimmunol 124, 83-92 (2002).
29. Rogers, J., Luber-Narod, J., Styren, S. D. & Civin, W. H. Expression of immune system-associated antigens by cells of the human central nervous system: relationship to the pathology of Alzheimer's disease. Neurobiol Aging 9, 339-349 (1988).
30. Malm, T. M., et al. Bone-marrow-derived cells contribute to the recruitment of microglial cells in response to beta-amyloid deposition in APP/PS1 double transgenic Alzheimer mice. Neurobiol Dis 18, 134-142 (2005).
31. Simard, A. R., Soulet, D., Gowing, G., Julien, J. P. & Rivest, S. Bone marrow-derived microglia play a critical role in restricting senile plaque formation in Alzheimer's disease. Neuron 49, 489-502 (2006).
32. El Khoury, J., et al. Ccr2 deficiency impairs microglial accumulation and accelerates progression of Alzheimer-like disease. Nat Med 13, 432-438 (2007).
33. Monsonego, A., et al. Increased T cell reactivity to amyloid beta protein in older humans and patients with Alzheimer disease. J Clin Invest 112, 415-422 (2003).
34. Kaku, M., et al. Amyloid beta protein deposition and neuron loss in osteopetrotic (op/op) mice. Brain Res Brain Res Protoc 12, 104-108 (2003).
35. Cheadle, C., Vawter, M. P., Freed, W. J. & Becker, K. G. Analysis of microarray data using Z score transformation. J Mol Diagn 5, 73-81 (2003).
36. Storey, J. D. & Tibshirani, R. Statistical significance for genomewide studies. Proc Natl Acad Sci USA 100, 9440-9445 (2003).
37. Zhao, Y. & Karypis, G. Clustering in life sciences. Methods Mol Biol 224, 183-218 (2003).
38. Bair, E. & Tibshirani, R. Semi-supervised methods to predict patient survival from gene expression data. PLoS Biol 2, E108 (2004).
39. Dettling, M. BagBoosting for tumor classification with gene expression data. Bioinformatics 20, 3583-3593 (2004).
40. Zhang, B., Kirov, S. & Snoddy, J. WebGestalt: an integrated system for exploring gene sets in various biological contexts. Nucleic Acids Res 33, W741-748 (2005).
41. Dennis, G., Jr., et al. DAVID: Database for Annotation, Visualization, and Integrated Discovery. Genome Biol 4, P3 (2003).
42. de Hoon, M. J., Imoto, S., Nolan, J. & Miyano, S. Open source clustering software. Bioinformatics 20, 1453-1454 (2004).
43. Saldanha, A. J. Java Treeview—extensible visualization of microarray data. Bioinformatics 20, 3246-3248 (2004).

### FIGURE LEGENDS

FIG. 1: Study outline. A total of 223 plasma samples were separated into a training set and two test sets. The AD patients and NDC subjects samples in the training set and the test set “AD” originate from the same seven medical centers and were evenly split by sex, age and cognitive scores. Changes in relative signaling protein concentrations were initially explored with “Significance Analysis of Microarray” (SAM) followed by cluster analysis. To discover predictors for classification the training set was analyzed by “Prediction Analysis of Microarray” (PAM). Then, PAM was set to class prediction mode and the predictors were used to classify the samples in the independent test set “AD”. Class prediction of pre-symptomatic AD was performed on samples from patients who were diagnosed with MCI at the date of blood draw (Test set “MCI”). Note that none of the samples from the test sets were used for any part of the predictor discovery process.

FIG. 2: SAM analysis and clustering of training set samples. Normalized array measurements of 120 plasma signaling proteins in the training set (43 AD patients, orange; 40 NDC, blue) were analyzed by SAM for differential relative protein concentrations. 19 proteins obtained a significant d-score (q-values ≦3.4%) and are presented in a heat map generated by an unsupervised cluster algorithm. Samples are arranged in columns and proteins in rows. Increased expression in AD versus NDC is shown in shades of red, reduced expression in shades of green, median expression is shown in black. Samples are clustered into AD and NDC indicated by the 1st order branches of the dendrogram (two black bars at the top).

FIGS. 3, 4A, 4B: Class prediction of clinically diagnosed AD. (FIG. 3) Predictor discovery in PAM was performed with normalized array measurements of 120 signaling proteins in a training set of 43 AD and 40 NDC plasma samples. In training (green line) and internal cross-validation (red line) decreasing the centroid threshold (x-axis) resulted in an increasing number of markers (inserted x-axis) that were used for classification and calculation of the classification error (y-axis). This led to the discovery of a minimal set of 18 predictors with lowest possible classification error. (FIGS. 4A and 4B) Predictive utility of the 18 predictors in the training set (FIG. 4A) and the blinded test set “AD” (FIG. 4B). Note the high sensitivity (Sens) and specificity (Spec) in predicting AD and non-AD class, respectively in the blinded test set. Acc, accuracy; PPV, positive predictive value; NPV, negative predictive value; LR, likelihood ratio. 95% confidence intervals are given in parenthesis and p-value was calculated with Fisher's exact test for the columns and rows in the 2×2 contingency table. For the two-class prediction analysis in c) NDC and OD were combined in one group.

FIG. 4C: Class prediction of pre-symptomatic AD in MCI patients. The 18 predictors obtained with PAM in the training set were used for AD and non-AD class prediction in normalized array measurements of 120 signaling proteins in plasma samples from 48 MCI patients (PAM class prediction). After an initial diagnosis (time point 0 diagnosis) these MCI patients were followed longitudinally and converted to AD, developed OD, or remained MCI (Follow-up diagnosis). Average conversion time in months with standard deviation is given over the arrows. AD and non-AD class were predicted with high accuracy (Acc), sensitivity (Sens) and specificity (Spec). Note that all eight MCI patients who later developed OD were predicted as non-AD class. PPV; positive predictive value; NPV, negative predictive value; LR, likelihood ratio. 95% confidence intervals are given in parenthesis and p-value was calculated with Fisher's exact test for the columns and rows in the 2×2 contingency table. MCI and OD of the follow-up diagnosis were combined in one group for this two-class prediction analysis.

FIGS. 5A and 5B: Biological and functional profiling of the 18 predictors. (FIG. 5A) Analysis of significant enrichment of the 18 predictors in human gene ontology (GO) categories. A Direct Acyclic Graph (DAG) generated in WebGestalt illustrates the significant enrichment for 7 out of 18 classificatation markers in two human GO sub-categories. Significance (p≦0.05) was calculated by the hypergeometric test. (FIG. 5B) Clustering of significant enrichment of the 18 predictors in biological pathways. Pathways with at least three observed proteins were found for 9 out of 18 predictors. The column “n genes” indicates the number of genes from our 120-protein reference list that are present (P) in a certain pathway, for which a change of expression can be expected (E; given as P×18/120) in this pathway, or which were observed (0) to be changed. Significance (p≦0.05) of expected versus obtained proteins was calculated by hypergeometric test in WebGestalt. DAVID 2006 clustered similar pathways into the same functional group and used EASE scores and geometric means of EASE scores (EASE geom) for ranking the pathways within a functional group and the functional groups, respectively. To illustrate the impact of the protein expression levels on a particular pathway, a relative pathway score was calculated as the sum of the d-scores obtained in SAM for the individual markers. BC; BioCarta, KEGG; Kyoto Encyclopedia of Genes and Genome.

### Supplementary Information, Ray et al.

### Supplementary Tables

TABLE 3
Proteins measured with cytokine antibody array

| | Entrez | Official gene name provided by HUGO | AD/NDC | AD/OND/RA | PAM |
|---|---|---|---|---|---|
| Protein Name | GeneID | Gene Nomenclature Committee (HGNC) | SAM19^{a} | SAM43^{b} | Predictors^{c} |
| Adiponectin | 9370 | adiponectin, C1Q and collagen domain | | x | |
| | | containing | | | |
| AGRP | 181 | agouti related protein homolog (mouse) | | x | |
| Amphiregulin | 374 | amphiregulin (schwannoma-derived growth | | | |
| | | factor) | | | |
| Ang-2 | 285 | angiopoietin 2 | x | | x |
| Angiogenin | 283 | angiogenin, ribonuclease, RNase A family, 5 | | | |
| Axl | 558 | AXL receptor tyrosine kinase | | | |
| basic FGF | 2247 | fibroblast growth factor 2 (basic) | | | |
| BDNF | 627 | brain-derived neurotrophic factor | | | |
| BMP-4 | 652 | bone morphogenetic protein 4 | | | |
| BMP-6 | 654 | bone morphogenetic protein 6 | | | |
| BTC | 685 | Betacellulin | | | |
| CCL1/I-309 | 6346 | chemokine (C-C motif) ligand 1 | | | |
| CCL2/MCP-1 | 6347 | chemokine (C-C motif) ligand 2 | | | |
| CCL3/MIP-1a | 6348 | chemokine (C-C motif) ligand 3 | | x | |
| CCL4/MIP-1b | 6351 | chemokine (C-C motif) ligand 4 | | | |
| CCL5/RANTES | 6352 | chemokine (C-C motif) ligand 5 | x | | x |
| CCL7/MCP-3 | 6354 | chemokine (C-C motif) ligand 7 | x | x | x |
| CCL8/MCP-2 | 6355 | chemokine (C-C motif) ligand 8 | | x | |
| CCL11/Eotaxin | 6356 | chemokine (C-C motif) ligand 11 | | | |
| CCL13/MCP-4 | 6357 | chemokine (C-C motif) ligand 13 | | | |
| CCL15/MIP-1d | 6359 | chemokine (C-C motif) ligand 15 | x | x | x |
| CCL16/HCC-4 | 6360 | chemokine (C-C motif) ligand 16 | | | |
| CCL17/TARC | 6361 | chemokine (C-C motif) ligand 17 | | x | |
| CCL18/PARC | 6362 | chemokine (C-C motif) ligand 18 | x | | x |
| | | (pulmonary and activation-regulated) | | | |
| CCL19/MIP-3b | 6363 | chemokine (C-C motif) ligand 19 | | | |
| CCL20/MIP-3a | 6364 | chemokine (C-C motif) ligand 20 | | | |
| CCL22/MDC | 6367 | chemokine (C-C motif) ligand 22 | x | x | |
| CCL23/CKb8-1 | 6368 | chemokine (C-C motif) ligand 23 | | | |
| CCL24/Eotaxin-2 | 6369 | chemokine (C-C motif) ligand 24 | | | |
| CCL25/TECK | 6370 | chemokine (C-C motif) ligand 25 | | | |
| CCL26/Eotaxin-3 | 10344 | chemokine (C-C motif) ligand 26 | | x | |
| CCL27/CTACK | 10850 | chemokine (C-C motif) ligand 27 | | | |
| CNTF | 1270 | ciliary neurotrophic factor | | | |
| CX3CL1/Fractalkine | 6376 | chemokine (C—X3—C motif) ligand 1 | | | |
| CXCL1,2,3/GRO- | 2919 | chemokine (C—X—C motif) ligand 1 | | | |
| α,β,γ | 2920 | chemokine (C—X—C motif) ligand 2 | | | |
| | 2921 | chemokine (C—X—C motif) ligand 3 | | | |
| CXCL1/GRO-a | 2919 | chemokine (C—X—C motif) ligand 1 | | | |
| CXCL5/ENA-78 | 6374 | chemokine (C—X—C motif) ligand 5 | | | |
| CXCL6/GCP-2 | 6372 | chemokine (C—X—C motif) ligand 6 | | | |
| | | (granulocyte chemotactic protein 2) | | | |
| CXCL7/NAP-2 | 5473 | pro-platelet basic protein (chemokine (C-X- | | | |
| | | C motif) ligand 7) | | | |
| CXCL8/IL-8 | 3576 | interleukin 8 | x | x | x |
| CXCL9/MIG | 4283 | chemokine (C—X—C motif) ligand 9 | | | |
| CXCL11/I-TAC | 6373 | chemokine (C—X—C motif) ligand 11 | | | |
| CXCL12/SDF-1 | 6387 | chemokine (C—X—C motif) ligand 12 (stromal | | x | |
| | | cell-derived factor 1) | | | |
| CXCL13/BLC | 10563 | chemokine (C—X—C motif) ligand 13 (B-cell | | | |
| | | chemoattractant) | | | |
| EGF | 1950 | epidermal growth factor (beta-urogastrone) | x | | x |
| EGFR | 1956 | epidermal growth factor receptor | | | |
| | | (erythroblastic leukemia viral (v-erb-b) | | | |
| | | oncogene homolog, avian) | | | |
| Fas | 355 | Fas (TNF receptor superfamily, member 6) | | x | |
| FGF-4 | 2249 | fibroblast growth factor 4 (heparin secretory | | | |
| | | transforming protein 1) | | | |
| FGF-6 | 2251 | fibroblast growth factor 6 | | x | |
| FGF-7 | 2252 | fibroblast growth factor 7 (keratinocyte | | | |
| | | growth factor) | | | |
| FGF-9 | 2254 | fibroblast growth factor 9 (glia-activating | | | |
| | | factor) | | | |
| Fit-3L | 2323 | fms-related tyrosine kinase 3 ligand | | | |
| G-CSF | 1440 | colony stimulating factor 3 (granulocyte) | x | | x |
| GDNF | 2668 | glial cell derived neurotrophic factor | x | x | x |
| GITR | 8784 | tumor necrosis factor receptor superfamily, | | | |
| | | member 18 | | | |
| GITR-L | 8995 | tumor necrosis factor (ligand) superfamily, | | | |
| | | member 18 | | | |
| GM-CSF | 1437 | colony stimulating factor 2 (granulocyte- | | x | |
| | | macrophage) | | | |
| HGF | 3082 | hepatocyte growth factor (hepapoietin A; | | x | |
| | | scatter factor) | | | |
| ICAM-1 | 3383 | intercellular adhesion molecule 1 (CD54), | x | x | x |
| | | human rhinovirus receptor | | | |
| ICAM-3 | 3385 | intercellular adhesion molecule 3 | | | |
| IFN-g | 3458 | interferon, gamma | | x | |
| IGF1-R | 3480 | insulin-like growth factor 1 receptor | | | |
| IGFBP-1 | 3484 | insulin-like growth factor binding protein 1 | | | |
| IGFBP-2 | 3485 | insulin-like growth factor binding protein 2 | | | |
| IGFBP-3 | 3486 | insulin-like growth factor binding protein 3 | | | |
| IGFBP-4 | 3487 | insulin-like growth factor binding protein 4 | | x | |
| IGFBP-6 | 3489 | insulin-like growth factor binding protein 6 | x | | x |
| IGF-I | 3479 | insulin-like growth factor 1 (somatomedin | | x | |
| | | C) | | | |
| IL-1 R-like 1 | 9173 | interleukin 1 receptor-like 1 | | | |
| IL-1 sRI | 3554 | interleukin 1 receptor, type I | | | |
| IL-1ra | 3557 | interleukin 1 receptor antagonist | | | |
| IL-1a | 3552 | interleukin 1, alpha | x | x | x |
| IL-1b | 3553 | interleukin 1, beta | | x | |
| IL-2 | 3558 | interleukin 2 | | | |
| IL-2 sRa | 3559 | interleukin 2 receptor, alpha | | | |
| IL-3 | 3562 | interleukin 3 (colony-stimulating factor, | x | x | x |
| | | multiple) | | | |
| IL-4 | 3565 | interleukin 4 | | x | |
| IL-5 | 3567 | interleukin 5 (colony-stimulating factor, | | x | |
| | | eosinophil) | | | |
| IL-6 | 3569 | interleukin 6 (interferon, beta 2) | | x | |
| IL-6 sR | 3570 | interleukin 6 receptor | | | |
| IL-7 | 3574 | interleukin 7 | | | |
| IL-10 | 3586 | interleukin 10 | | x | |
| IL-11 | 3589 | interleukin 11 | x | | x |
| IL-12p40 | 3593 | interleukin 12B (natural killer cell | | x | |
| | | stimulatory factor 2, p40) | | | |
| IL-12p70 | 3592 | interleukin 12A (natural killer cell | | | |
| | | stimulatory factor 1, p35) | | | |
| IL-13 | 3596 | interleukin 13 | | x | |
| IL-15 | 3600 | interleukin 15 | | x | |
| IL-16 | 3603 | interleukin 16 (lymphocyte chemoattractant | | | |
| | | factor) | | | |
| IL-17 | 3605 | interleukin 17A | | | |
| Leptin | 3952 | Leptin | | x | |
| LIGHT | 8740 | tumor necrosis factor (ligand) superfamily, | | x | |
| | | member 14 | | | |
| M-CSF | 1435 | colony stimulating factor 1 (macrophage) | x | x | x |
| MIF | 4282 | macrophage migration inhibitory factor | | x | |
| | | (glycosylation-inhibiting factor) | | | |
| MSP a-chain | 4485 | macrophage stimulating 1 (hepatocyte | | | |
| | | growth factor-like) alpha-chain | | | |
| NGF-b | 4803 | nerve growth factor, beta polypeptide | | | |
| NT-3 | 4908 | neurotrophin 3 | | | |
| NT-4/5 | 4909 | neurotrophin 5 (neurotrophin 4/5) | | | |
| oncostatin M | 5008 | oncostatin M | | | |
| Osteoprotegerin | 4982 | tumor necrosis factor receptor superfamily, | | x | |
| | | member 11b | | | |
| PDGF-BB | 5155 | platelet-derived growth factor beta | x | x | x |
| | | polypeptide (simian sarcoma viral (v-sis) | | | |
| | | oncogene homolog) | | | |
| PLGF | 5228 | placental growth factor, vascular endothelial | | | |
| | | growth factor-related protein | | | |
| SCF | 4254 | KIT ligand | | | |
| Sgp130 | 3572 | interleukin 6 signal transducer (gp130, | | | |
| | | oncostatin M receptor) | | | |
| TGF-b1 | 7040 | transforming growth factor, beta 1 | | x | |
| TGF-b3 | 7043 | transforming growth factor, beta 3 | | | |
| TIMP-1 | 7076 | TIMP metallopeptidase inhibitor 1 | | | |
| TIMP-2 | 7077 | TIMP metallopeptidase inhibitor 2 | | | |
| TNFR-1 | 7132 | tumor necrosis factor receptor superfamily, | | | |
| | | member 1A | | | |
| TNFR-2 | 8764 | tumor necrosis factor receptor superfamily, | | | |
| | | member 14 | | | |
| TNF-a | 7124 | tumor necrosis factor (TNF superfamily, | x | x | x |
| | | member 2) | | | |
| TNF-b | 4049 | lymphotoxin alpha (TNF superfamily, | | x | |
| | | member 1) | | | |
| Tpo | 7066 | thrombopoietin (megakaryocyte growth and | | x | |
| | | development factor) | | | |
| TRAIL R3 | 8794 | tumor necrosis factor receptor superfamily, | | x | |
| | | member 10c, decoy without an intracellular | | | |
| | | domain | | | |
| TRAIL R4 | 8793 | tumor necrosis factor receptor superfamily, | x | x | x |
| | | member 10d, decoy with truncated death | | | |
| | | domain | | | |
| Tyro3 | 7301 | TYRO3 protein tyrosine kinase | | | |
| uPAR | 5329 | plasminogen activator, urokinase receptor | | | |
| VEGF-B | 7423 | vascular endothelial growth factor B | | x | |
| VEGF-D | 2277 | c-fos induced growth factor (vascular | | | |
| | | endothelial growth factor D) | | | |
| XCL1/Lymphotactin | 6375 | chemokine (C motif) ligand 1 | | | |

^{a}19 proteins identified in test set comparing AD vs NDC with SAM analysis (FIG. 2)
^{b}43 proteins identified in SAM analysis comparing AD vs OND and RA (FIG. 7A)
^{c}18 proteins identified in test set with PAM predictor discovery algorithm (FIG. 3)

TABLE 4
Subjects' characteristics

| | | Age | Sex | MMSE^{a} |
|---|---|---|---|---|
| Clinical Diagnosis | Number | (mean ± SD) | (% female) | (mean ± SD) |
| Alzheimer disease (AD) | 85 | 76.2 ± 78^{b} | 47 | 16.3 ± 7.9 |
| Non-demented controls (NDC) | 79 | 71.4 ± 7.8^{b} | 40 | 29.4 ± 1.0 |
| Training set | | | | |
| AD | 43 | 74.3 ± 8.8 | 44 | 16.2 ± 8.0 |
| NDC | 40 | 72.3 ± 7.8 | 35 | 29.3 ± 0.7 |
| Test set | | | | |
| AD | 42 | 78.2 ± 6.4 | 33 | 17.1 ± 7.5 |
| NDC | 39 | 70.7 ± 7.7 | 45 | 29.4 ± 1.1 |
| Other dementia (OD) | 11 | | | |
| Frontotemporal dementia (FTD) | 8 | 64.5 ± 9.1 | 33 | 19.7 ± 9.8 |
| Corticobasal degeneration (CBD) | 3 | 60.0 ± 5.3 | 66 | 17.0 ± 6.6 |
| Mild cognitive impairment (MCI) | 48 | 71.3 ± 8.1 | 55 | 27.3 ± 1.9 |
| MCI→AD^{c} | 23 | 75.3 ± 5.1 | 61 | 27.4 ± 1.8 |
| MCI→OD^{c} | 8 | 75.2 ± 8.2 | 13 | 27.0 ± 1.6 |
| MCI→MCI^{c} | 17 | 65.8 ± 8.3 | 47 | 27.6 ± 2.0 |
| Other neurological disease (OND) | | | | |
| Parkinson's disease | 5 | 79.8 ± 3.1 | 0 | n.a. |
| ALS | 2 | 47.5 ± 12.0 | 0 | n.a. |
| Multiple sclerosis | 2 | 55 ± 0.0 | 100 | n.a. |
| Peripheral neuropathy | 12 | 69.5 ± 8.2 | 41 | n.a. |
| Rheumatoid arthritis | 16 | 63.9 ± 12.0 | 6 | n.a. |

^{a}Mini-mental state exam¹.
^{b}Age difference between AD and NDC is not significant P = 0.25, Student's t-test.
^{c}Out of 48 patients diagnosed with MCI at blood draw, 23 converted to AD within 2-5 years (MCI→AD; average conversion time 29.6 ± 14.6 months), 8 converted to OD (MCI→OD; average conversion time 27.8 ± 1.6 months), whereas 17 were still diagnosed MCI 4-6 years later (MCI→MCI).
n.a., not available.

TABLE 5
Expression changes in AD reported for 7 of the 18 predictors

| | Our | | | |
|---|---|---|---|---|
| Protein | finding | Plasma/Serum | CSF | Brain Parenchyma |
| ICAM-1 | ↑ | ↑ sICAM-1 in serum² | | ↑ |
| | | | | Immunoreactivity |
| | | | | in and around |
| | | | | plaques in humans^{3,4}; |
| | | | | ↑ with |
| | | | | progression of disease |
| | | | | in activated microglia |
| | | | | and in plaques of APP |
| | | | | mice⁵ |
| CXCL8/ | ↑ | | ↑ in MCI and | ↑ in |
| IL-8 | | | AD⁶ | microvasculature⁷ |
| | | | | Reviewed by |
| | | | | Xia et al.⁸ |
| IGFBP-6 | ↑ | | ↑ in AD⁹ | |
| M-CSF | ↓ | | ↑ in AD¹⁰ | ↑ neuronal |
| | | | | immunoreactivity in |
| | | | | proximity to Aβ |
| | | | | deposits¹⁰ |
| | | | | ↑ |
| | | | | immunoreactivity |
| | | | | on neuritic structures |
| | | | | near Aβ deposits in |
| | | | | APP transgenic mice¹¹ |
| IL-1α^{a} | ↓ | [symbol] serum in AD and | | |
| | | multi-infarct dementia¹² | | |
| TNF-α | ↓ | ↑ in plasma in | ↑ in MCI¹⁸ | ↓ in AD frontal |
| | | centenarians with AD | ↑ in AD and | cortex, superior |
| | | compared to those without¹³ | vascular dementia¹⁹ | temporal gyrus , and |
| | | ↓ in serum in mild- | ↓ in AD²⁰ | entorhinal cortex |
| | | moderate AD versus severe | [symbol] AD vs | compared with controls¹⁷ |
| | | AD and vascular dementias¹⁴ | control¹⁷ | |
| | | ↓ in serum in early and | | |
| | | late onset AD versus control¹⁵ | | |
| | | ↓ in serum in AD and | | |
| | | multi-infarct dementia¹⁶; | | |
| | | [symbol] AD vs control¹⁷ | | |
| PDGF- | ↓ | | | ↓ number of |
| BB | | | | PDGF-BB |
| | | | | immunoreactive |
| | | | | pyramidal neurons in |
| | | | | AD²¹ |
| | | | | ↑ |
| | | | | immunoreactivity |
| | | | | with neurofibrillary |
| | | | | tangles in AD²¹ |

Changes in expression levels (RNA or protein) or abnormal presence of the listed proteins in plasma/serum, CSF, or brain parenchyma in human AD or AD mouse models reported in PubMed articles. APP, amyloid precursor protein; MCI, mild cognitive impairment; ↑, increased; ↓ decreased; [symbol] no change; empty cells, no reports found or proteins were reported to be undetectable.

^{a} 329 reports are found in PubMed with the search terms “alzheimer's interleukin-1alpha” but an extensive search of these articles failed to find evidence for changes in IL-1α. Instead, most articles report on Il-1β or IL1 We did not list any reports of genetic associations between the listed factors and AD. As reviewed recently, meta-analyses of multiple genetic studies have so far failed to produce any significant genetic effects of inflammatory genes²².

### SUPPLEMENTARY FIGURES

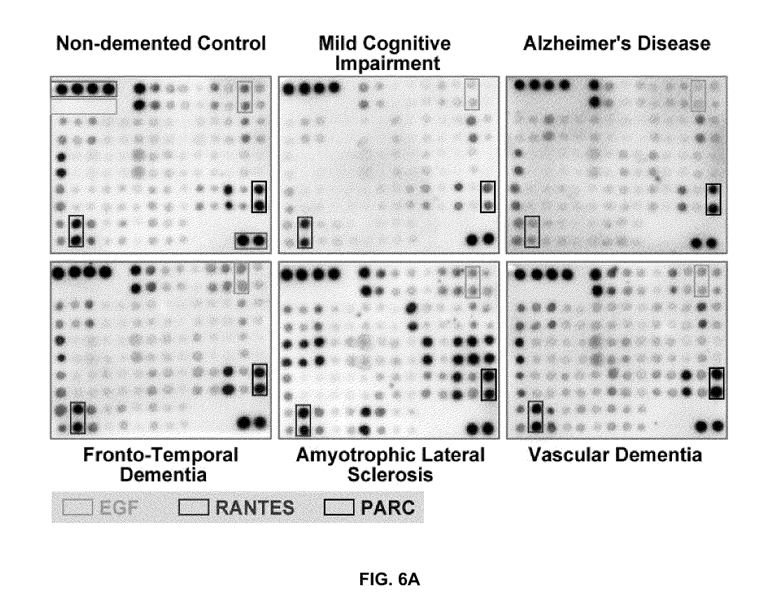

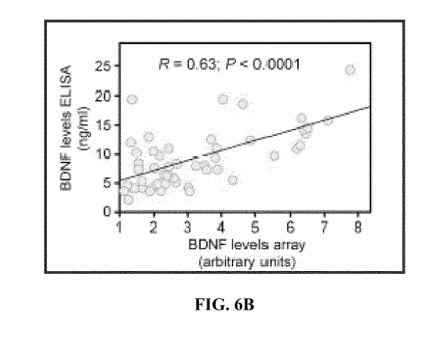

FIG. 6A: Array filter membrane. Examples of autoradiographs exposed to filters from patient samples with the indicated diagnoses. Plasma was incubated with an array membrane that detects 60 proteins. Each protein is measured in duplicates. Arrays were developed and exposed to autoradiographic film. Red boxes, positive controls; green box, negative controls. Colored boxes indicate the location of the detection of three proteins. Note the differences in expression patterns among the various conditions.

FIG. 6B: Example correlation of array results with ELISA results. Normalized array measurements for 50 samples were compared to ELISA measurements for aliquots of the same samples for the protein BDNF (brain-derived neurotrophic factor). The line represents best-fit. The R value and significance (P value) are also displayed.

FIG. 7A: Distinct pattern of signaling protein expression in AD compared with NDC. Normalized array measurements of 18 differentially expressed signaling proteins in plasma from 85 AD (orange), 79 NDC (blue) are shown in a heat map after unsupervised clustering. Samples are arranged in columns and proteins in rows. Increased expression in AD versus NDC is shown in shades of red, reduced expression in shades of green, median expression is shown in black. Samples are clustered into AD and NDC with high accuracy indicated by the 1^{st} order branches of the dendrogram (two black bars at the top).

FIG. 7B: Distinct signature of signaling protein expression in AD compared with other neurological diseases or rheumatoid arthritis. Normalized array measurements of 43 differentially expressed signaling proteins in plasma from 85 AD (orange), 21 other neurological diseases (OND, yellow), and 16 rheumatoid arthritis (RA; black) patients are shown in a heat map after unsupervised clustering. Samples are arranged in columns and proteins in rows. Increased expression in AD versus OND/RA is shown in shades of red, reduced expression in shades of green, median expression is shown in black. Samples are clustered into AD, RA and OND with high accuracy indicated by the 1^{5t} order branches of the dendrogram (three black bars at the top).

FIGS. 8A and 8B: Functional relationships between 18 predictors. (FIG. 8A) Heat map illustrating functional annotations of 18 classification markers found in PubMed. Colors indicate d-score as calculated by SAM in FIG. 2 representing increased (red) or decreased (green) expression in AD compared with NDC. Off white indicates no PubMed entry was found linking a given factor with the specified functions. A red or green entry means the specific factor modulates the indicated biological process or disease or is regulated by it. For most of the 18 signaling proteins it has been reported that they are produced in the CNS and some of them have been found in rodents to be transported (pink) or not (black) across the blood-brain-barrier (BBB). Additionally, several predictors have been implicated in aging (purple) or AD (orange). Note that nine of the predictors have never been associated with expression in the CNS or the periphery in AD before. For reports on expression changes in AD see Table 5. (FIG. 8B) Illustration of the impact of protein expression levels on a particular function. A relative function score was calculated as the sum of the d-scores obtained in SAM for the individual markers.

### SUPPLEMENTARY REFERENCES

1. Folstein, M. F., Folstein, S. E. & McHugh, P. R. “Mini-mental state”. A practical method for grading the cognitive state of patients for the clinician. J Psychiatr Res 12, 189-198 (1975).
2. Rentzos, M., et al. Serum levels of soluble intercellular adhesion molecule-1 and soluble endothelial leukocyte adhesion molecule-1 in Alzheimer's disease. J Geriatr Psychiatry Neurol 17, 225-231 (2004).
3. Rozemuller, J. M., Eikelenboom, P., Pals, S. T. & Stam, F. C. Microglial cells around amyloid plaques in Alzheimer's disease express leucocyte adhesion molecules of the LFA-1 family. Neurosci Lett 101, 288-292 (1989).
4. Verbeek, M. M., et al. Accumulation of intercellular adhesion molecule-1 in senile plaques in brain tissue of patients with Alzheimer's disease. Am J Pathol 144, 104-116 (1994).
5. Apelt, J., Lessig, J. & Schliebs, R. Beta-amyloid-associated expression of intercellular adhesion molecule-1 in brain cortical tissue of transgenic Tg2576 mice. Neurosci Lett 329, 111-115 (2002).
6. Galimberti, D., et al. Intrathecal chemokine synthesis in mild cognitive impairment and Alzheimer disease. Arch Neurol 63, 538-543 (2006).
7. Grammas, P., Samany, P. G. & Thirumangalakudi, L. Thrombin and inflammatory proteins are elevated in Alzheimer's disease microvessels: implications for disease pathogenesis. J Alzheimers Dis 9, 51-58 (2006).
8. Xia, M. Q. & Hyman, B. T. Chemokines/chemokine receptors in the central nervous system and Alzheimer's disease. J Neurovirol 5, 32-41 (1999).
9. Tham, A., et al. Insulin-like growth factors and insulin-like growth factor binding proteins in cerebrospinal fluid and serum of patients with dementia of the Alzheimer type. J Neural Transm Park Dis Dement Sect 5, 165-176 (1993).
10. Yan, S. D., et al. Amyloid-b peptide-receptor for advanced glycation endproduct interaction elicits neuronal expression of macrophage-colony stimulating factor: a proinflammatory pathway in Alzheimer disease. Proc. Natl. Acad. Sci. USA 94, 5296-5301 (1997).
11. Murphy, G. M., Jr., Zhao, F., Yang, L. & Cordell, B. Expression of macrophage colony-stimulating factor receptor is increased in the AbetaPP(V717F) transgenic mouse model of Alzheimer's disease. Am J Pathol 157, 895-904 (2000).
12. Cacabelos, R., Franco-Maside, A. & Alvarez, X. A. Interleukin-1 in Alzheimer's disease and multi-infarct dementia: neuropsychological correlations. Methods Find Exp Clin Pharmacol 13, 703-708 (1991).
13. Bruunsgaard, H., et al. A high plasma concentration of TNF-alpha is associated with dementia in centenarians. J Gerontol A Biol Sci Med Sci 54, M357-364 (1999).
14. Paganelli, R., et al. Proinflammatory cytokines in sera of elderly patients with dementia: levels in vascular injury are higher than those of mild-moderate Alzheimer's disease patients. Exp Gerontol 37, 257-263 (2002).
15. Alvarez, X. A., Franco, A., Fernandez-Novoa, L. & Cacabelos, R. Blood levels of histamine, IL-1 beta, and TNF-alpha in patients with mild to moderate Alzheimer disease. Mol Chem Neuropathol 29, 237-252 (1996).
16. Cacabelos, R., Alvarez, X. A., Franco-Maside, A., Fernandez-Novoa, L. & Caamano, J. Serum tumor necrosis factor (TNF) in Alzheimer's disease and multi-infarct dementia. Methods Find Exp Clin Pharmacol 16, 29-35 (1994).
17. Lanzrein, A. S., et al. Longitudinal study of inflammatory factors in serum, cerebrospinal fluid, and brain tissue in Alzheimer disease: interleukin-1beta, interleukin-6, interleukin-1 receptor antagonist, tumor necrosis factor-alpha, the soluble tumor necrosis factor receptors I and II, and alphal-antichymotrypsin. Alzheimer Dis Assoc Disord 12, 215-227 (1998).
18. Tarkowski, E., Andreasen, N., Tarkowski, A. & Blennow, K. Intrathecal inflammation precedes development of Alzheimer's disease. J Neurol Neurosurg Psychiatry 74, 1200-1205 (2003).
19. Tarkowski, E., Blennow, K., Wallin, A. & Tarkowski, A.h. Intracerebral production of tumor necrosis factor-alpha, a local neuroprotective agent, in Alzheimer disease and vascular dementia. J Clin Immunol 19, 223-230 (1999).
20. Richartz, E., et al. Decline of immune responsiveness: a pathogenetic factor in Alzheimer's disease? J Psychiatr Res 39, 535-543 (2005).
21. Masliah, E., Mallory, M., Alford, M., DeTeresa, R. & Saitoh, T. PDGF is associated with neuronal and glial alterations of Alzheimer's disease. Neurobiol. Aging 16, 549-556 (1995).
22. Wyss-Coray, T. Inflammation in Alzheimer disease: driving force, bystander or beneficial response? Nat Med 12, 1005-1015 (2006).

### Example 2

Further benefit from the findings of the filter array studies (18 predictive markers) would be realized if the assay were converted to a quantitative or semi-quantitative assay platform that is amenable to high sample throughput. SearchLight is a highly sensitive multiplex ELISA system utilizing a chemiluminescent signal readout. This platform was selected for initial evaluation since 16 of the 18 protein markers identified as predictive from the filter array studies were available commercially. 5 different multiplexes were generated by arraying the appropriate capture antibodies onto the wells of a microtiter plate. Multiplex configurations were based on historical data gathered by the manufacturer related to approximate sample dilution requirements and separation of individual reactions that demonstrate known undesirable interactions.

Following blocking of unreacted sites, plasma (or dilution thereof) was incubated. All samples (or various dilutions thereof) were run on the plate in duplicate. Following several washes, a cocktail of the appropriate detection antibodies, each chemically modified with biotin, was incubated. Following several washes, streptavidin-HRP (HRP=horseradish peroxidase) was incubated. Following washes, the enzymatic substrate was added. HRP converts the chemical substrate to a product that emits photons. Detection was achieved by capturing the image using a CCD camera (essentially taking a high quality picture). The data were extracted from the image using an appropriate software package that reads the light intensity as a number pixel by pixel and can integrate that for an entire spot. For each marker in the multiplex, a standard curve was generated by running serial dilutions of purified protein or protein cocktails of known concentrations on the same plate as the samples. The concentrations of the markers in the test samples were calculated by comparison to the standard curve for each marker. Various dilutions of the test sample may be used to ensure that the sample values fit onto the standard curve and can be accurately calculated.

For platform performance evaluation purposes, control materials were generated to represent strongly AD or strongly NDC signature patterns with sufficient volume for use in multiple assays. One approach to creating such control materials is to pool several samples together, thus increasing the volume of material available. Pooled materials are incubated at 4 C for 30 minutes prior to centrifugation at 10,000 g for 10 minutes to remove any aggregates or precipitates. Aliquots are prepared and frozen at −80 C for single-use to avoid repeated freeze-thaw cycles. The samples selected for inclusion in the pools were all collected from one center. These samples are pools of 8 AD plasma samples (pAD) and 8 NDC plasma samples (pNDC). 2 aliquots of each of the pools were analyzed.

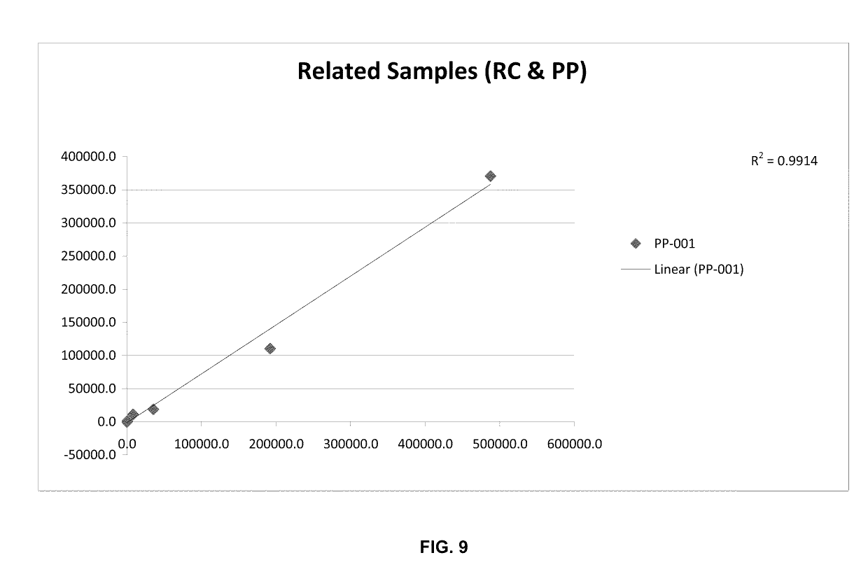

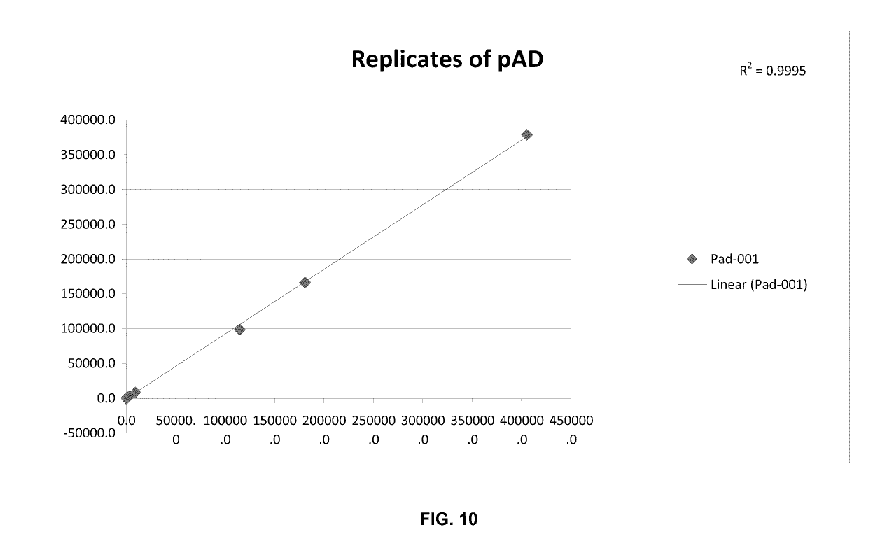

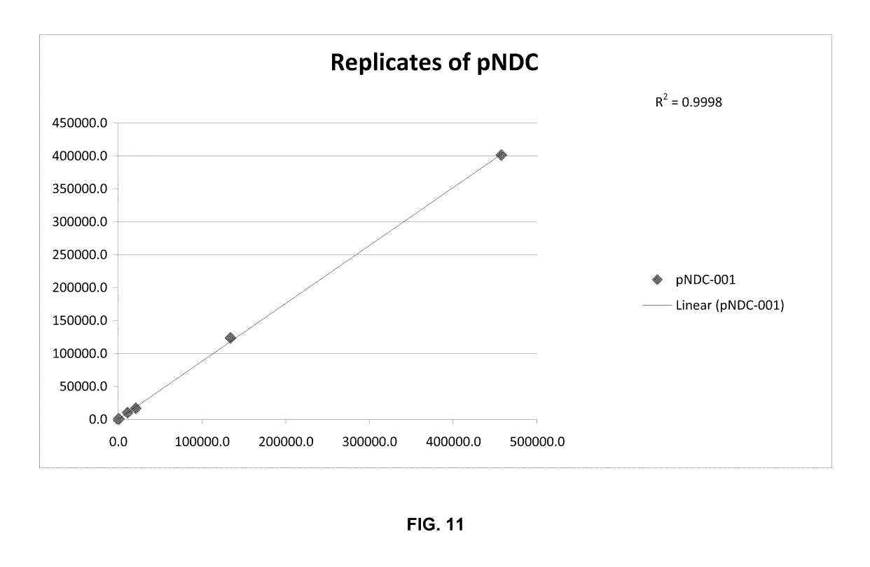

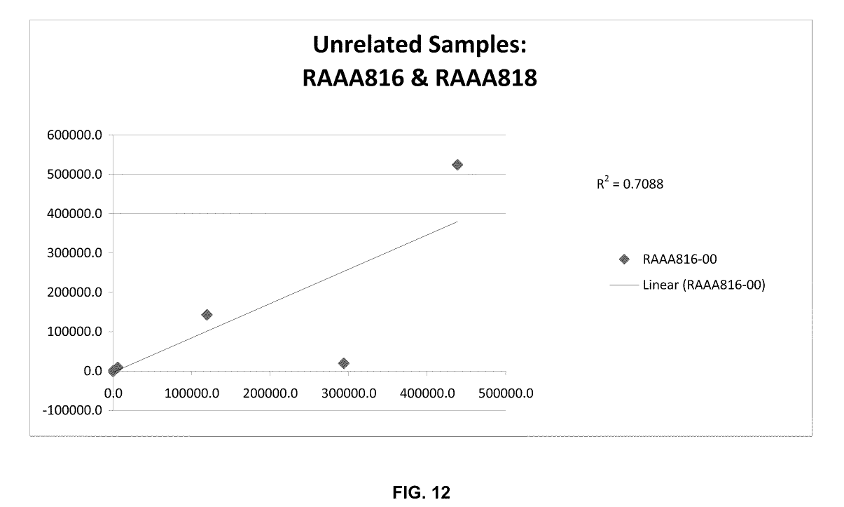

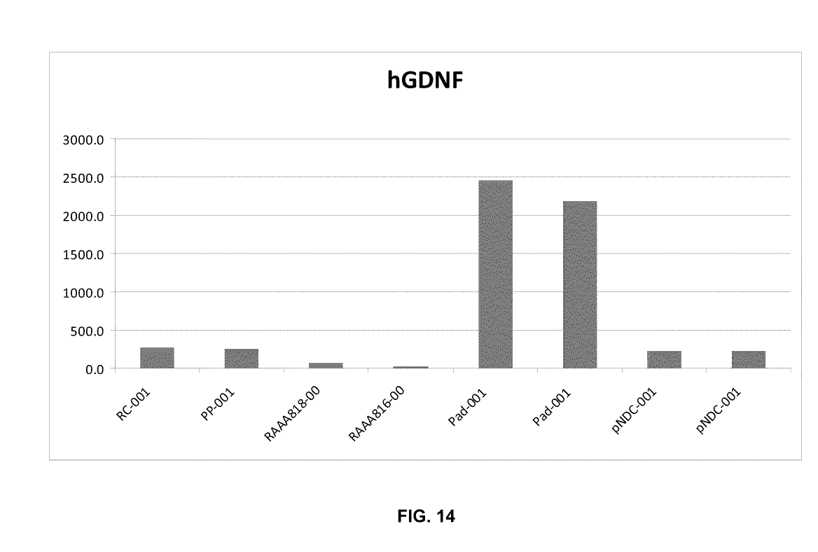

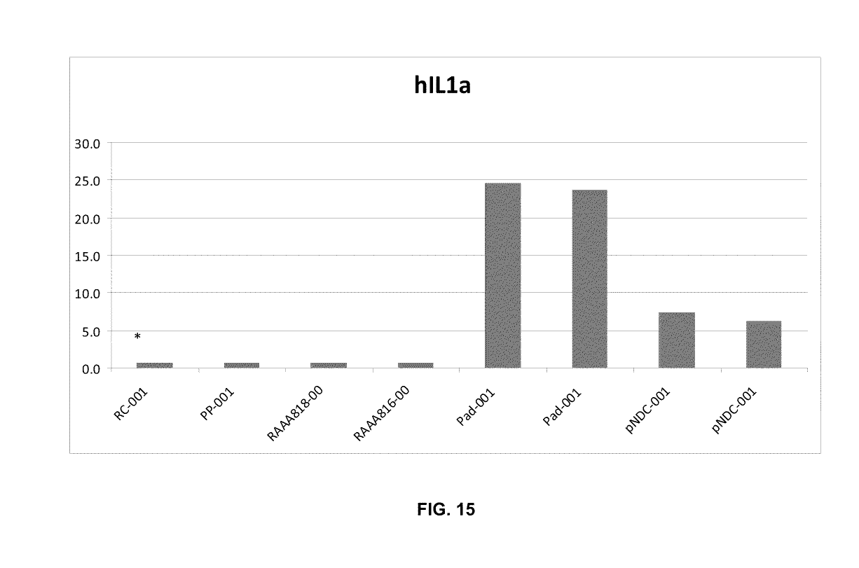

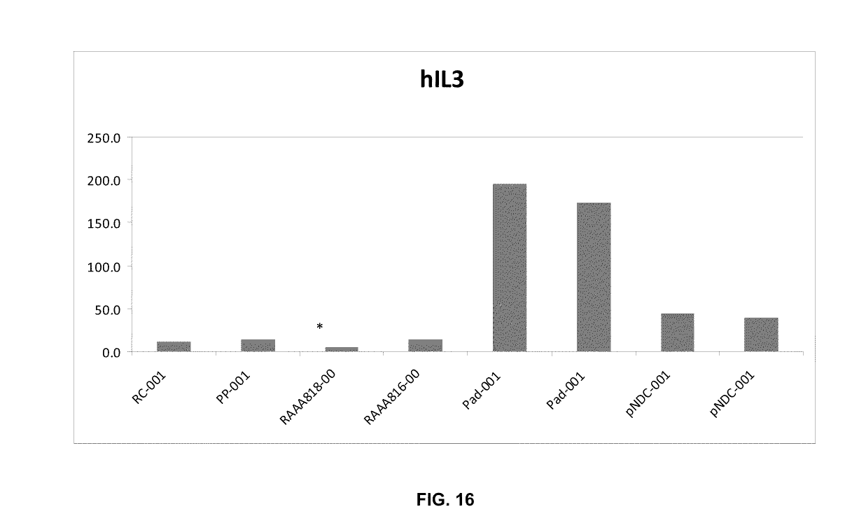

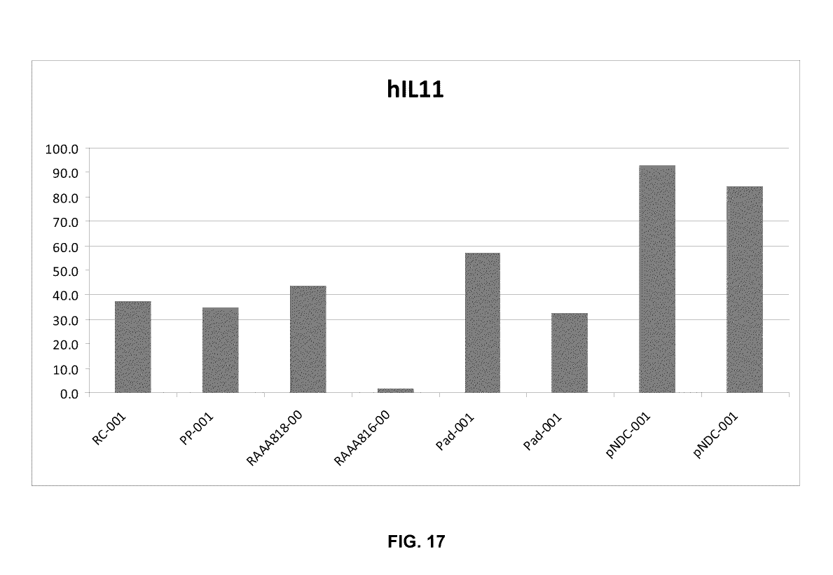

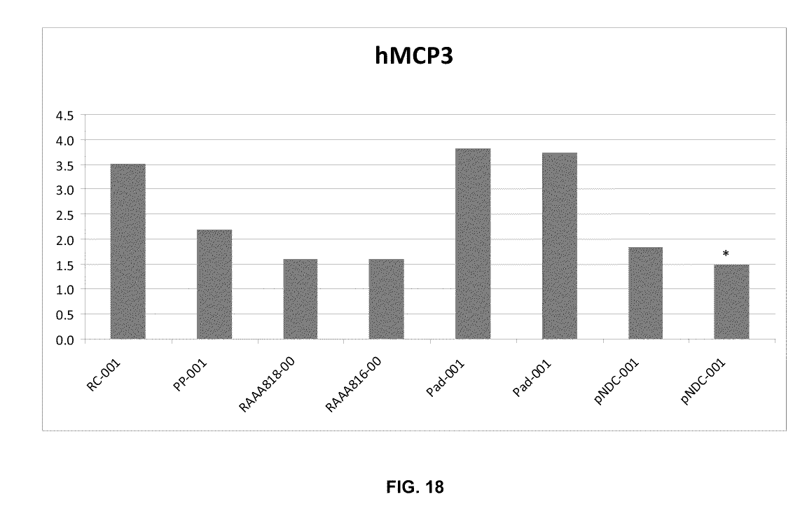

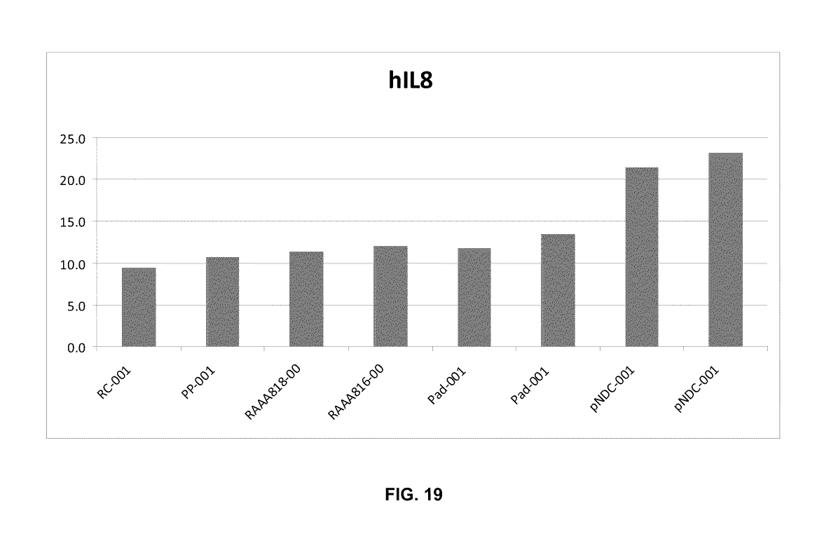

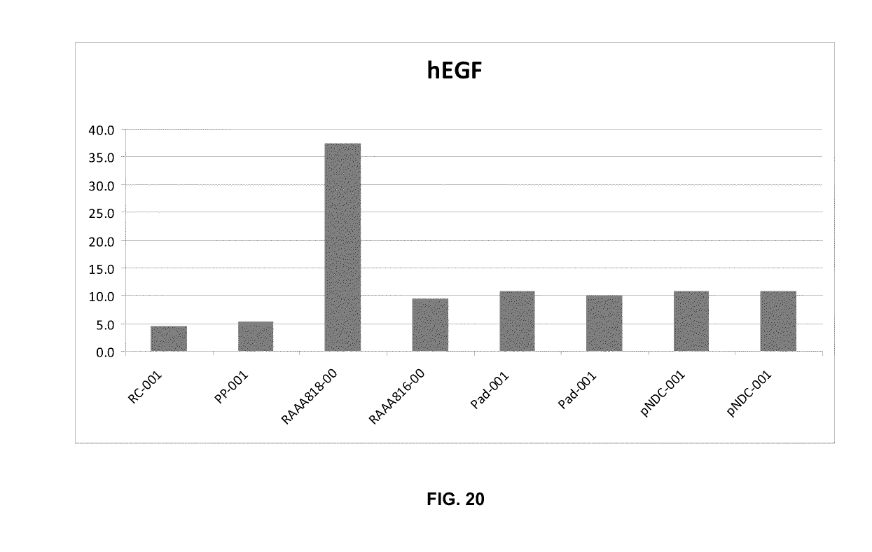

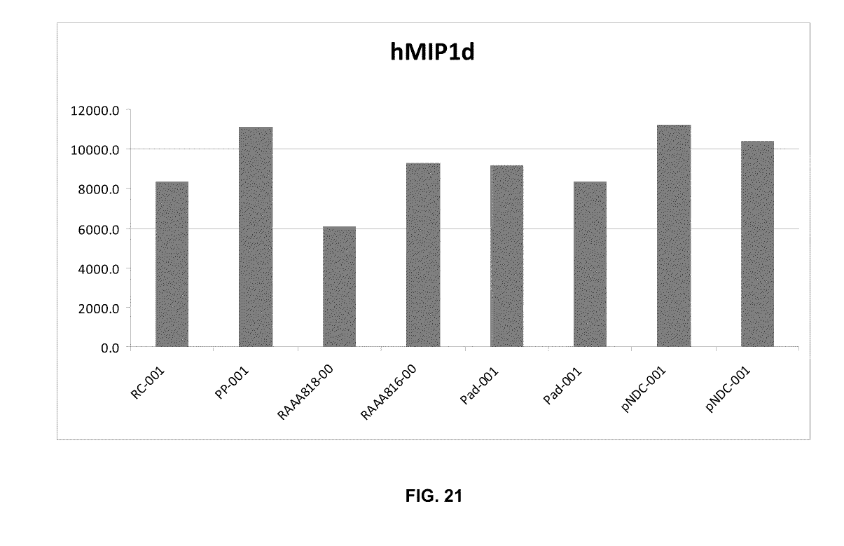

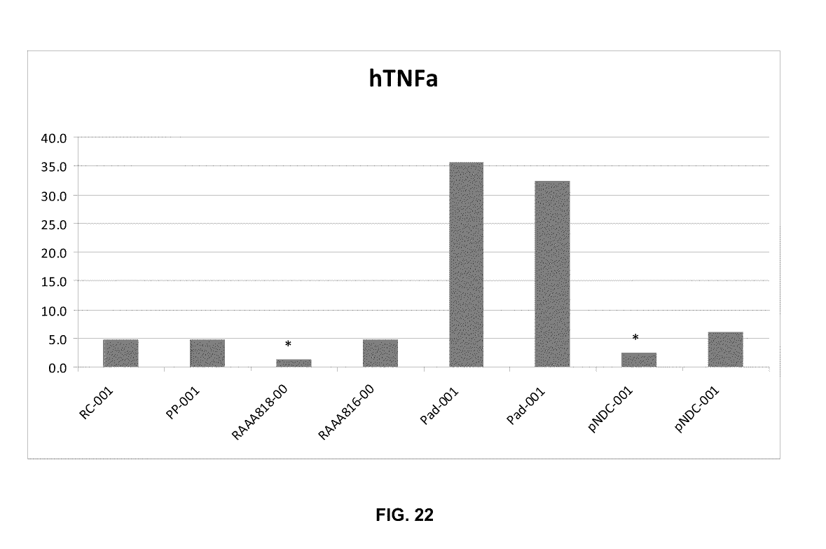

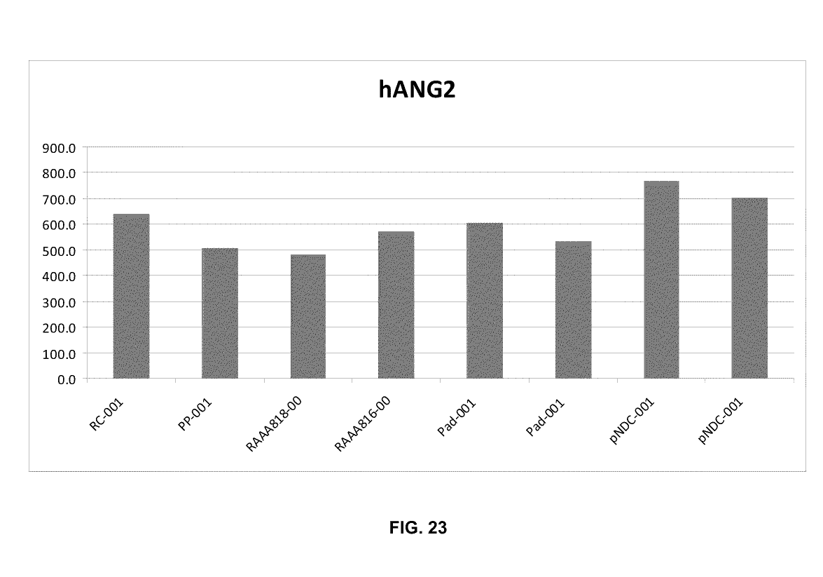

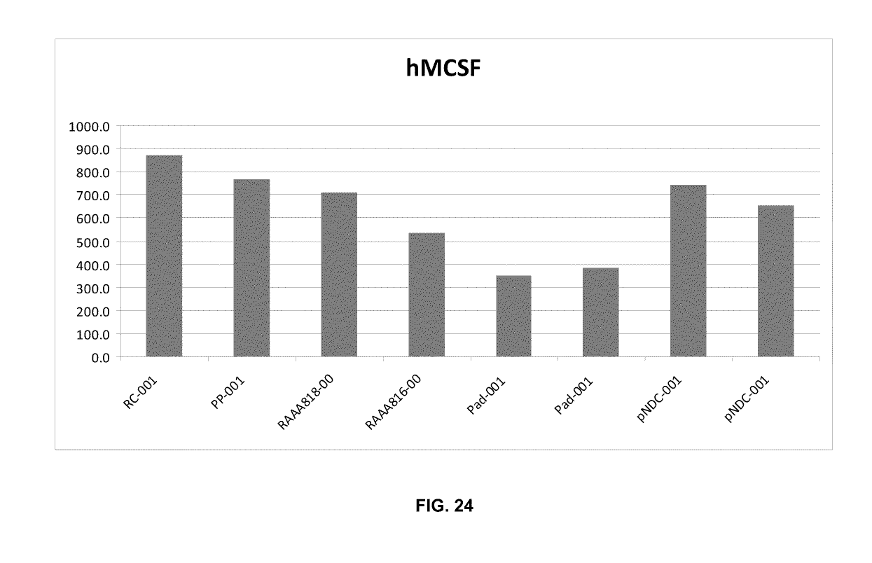

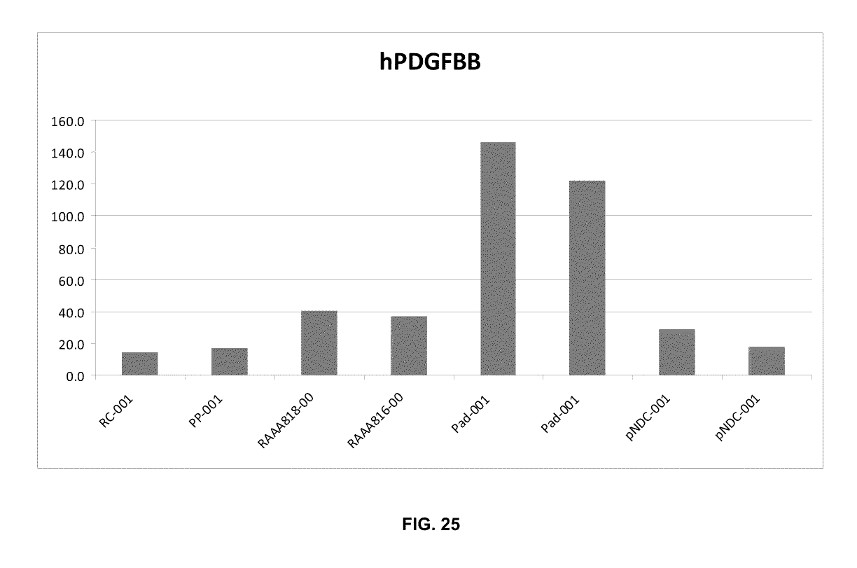

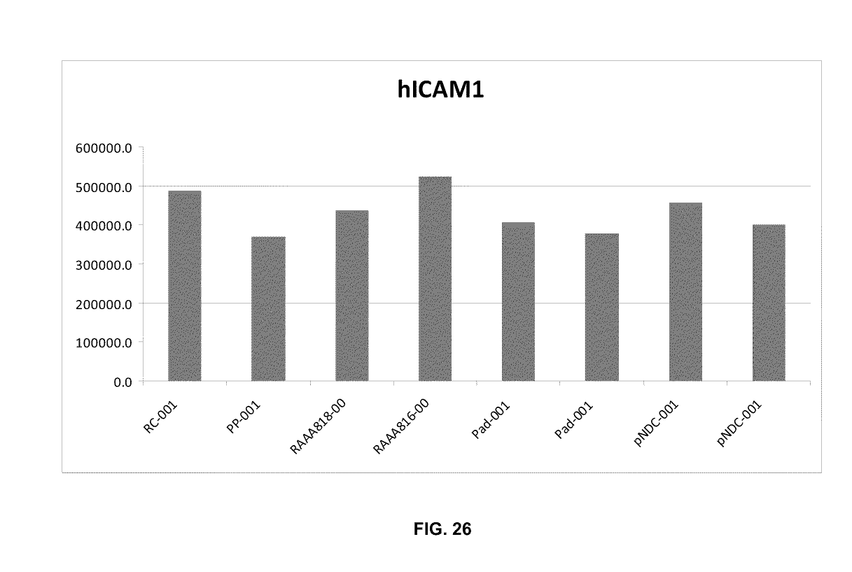

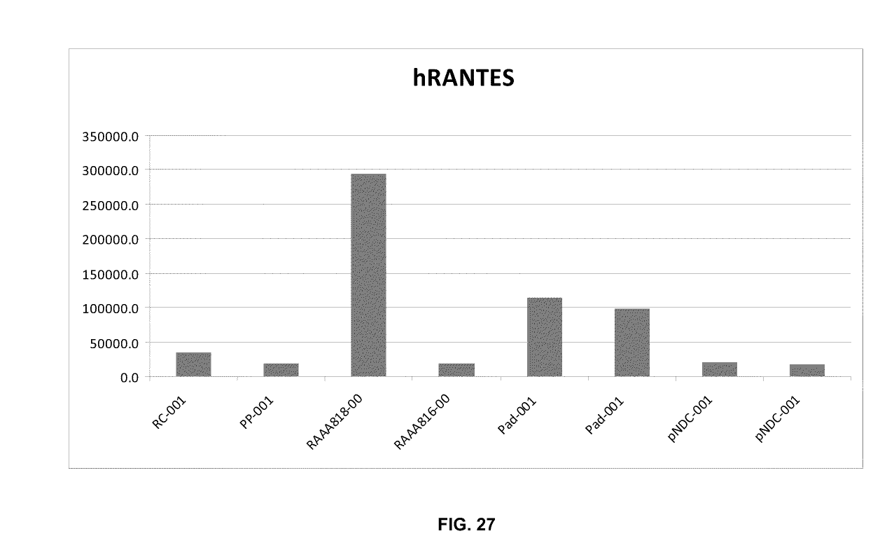

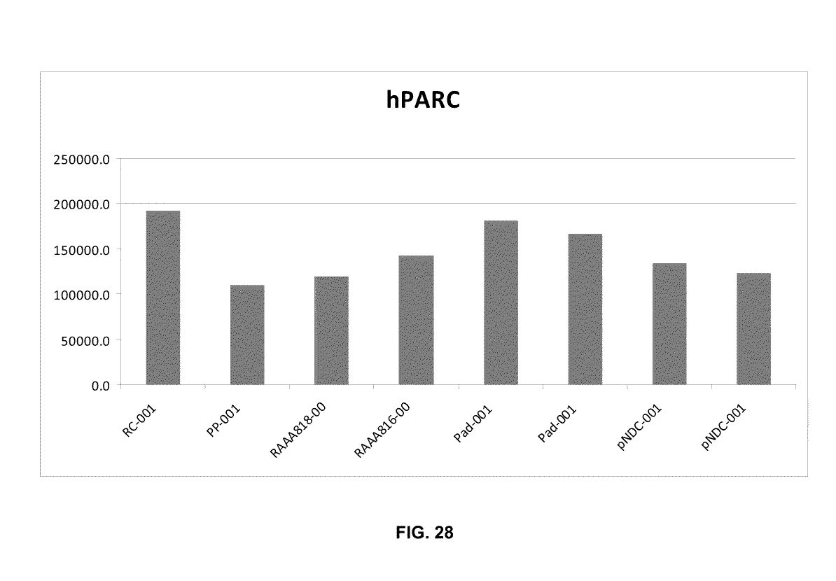

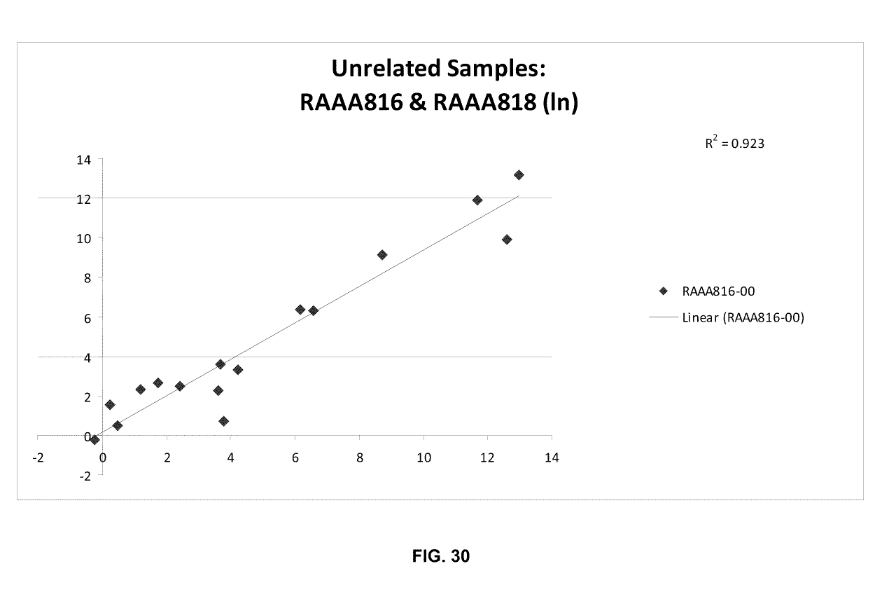

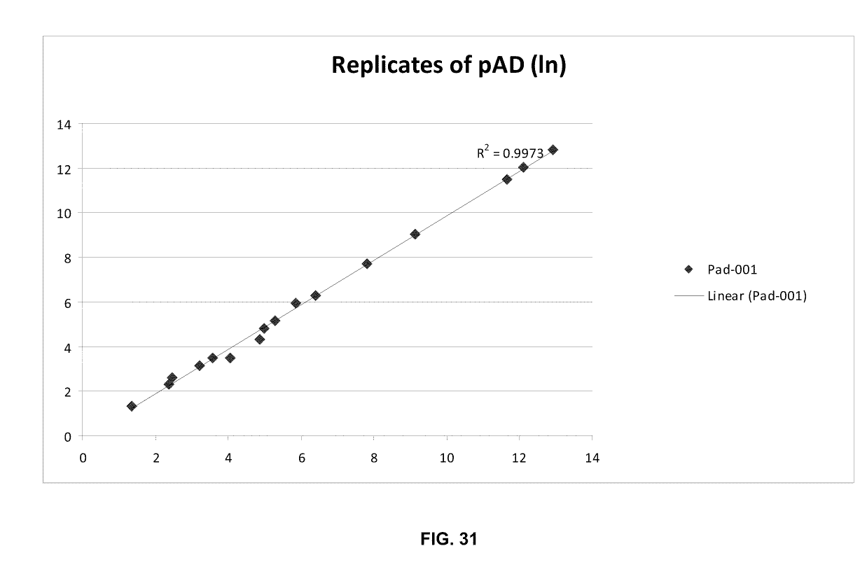

The results are shown in FIGS. 9-32 and Tables 6-9. Abbreviations: RC=register control; PP=pooled purchased control samples; pAD=pooled AD samples; pNDC=pooled non-dementia control samples; RAAA=test sample; “Avg”=average; “FC”=fold change, calculated by dividing the average pAD value by the average pNDC value.

Table 6 is an analysis of the control materials samples (replicate of other control samples eliminated). Fold change (FC) values reflecting a change of >20% (light grey) or <20% (dark grey) are highlighted. The results summarized in Table 6 demonstrate that 12 of the 16 markers showed greater than a 20% difference between the 2 pooled control material samples.

TABLE 6
Fold Change Determination of Control Materials

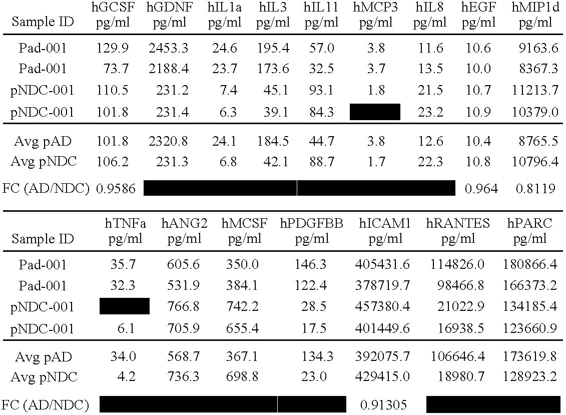

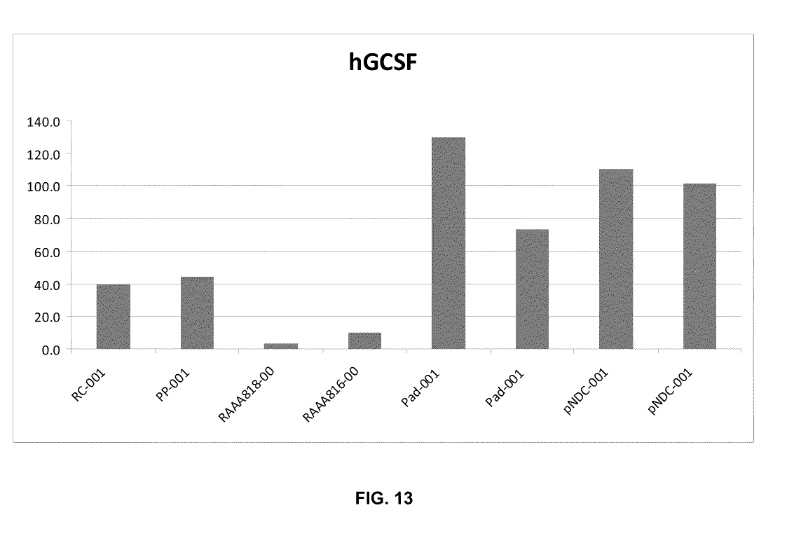

The cytokine concentration results for all samples tested are shown in Table 7 and are shown graphically for each marker in FIGS. 13-28. Correlations of replicated samples and related or unrelated samples are shown in tabular form in Table 8 and in graphical form in FIGS. 9-12. These demonstrate the high level of reproducibility of identical and highly related samples relative to that observed for unrelated samples.

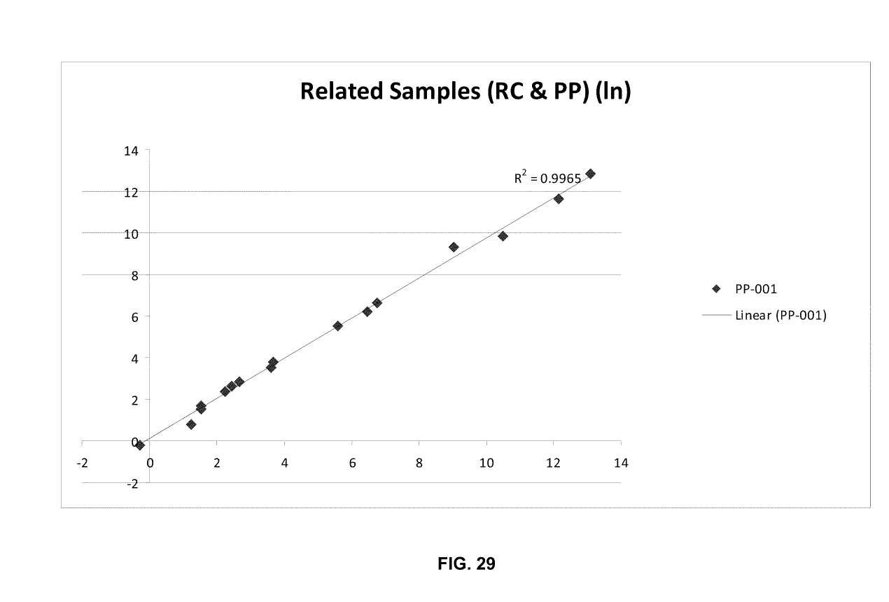

The correlations shown for the concentration data may be misleading given the large range of concentration values observed for the markers of interest. Such large ranges in concentrations can mask variation in the data. Converting the data to a more similar scale can be accomplished by transformation of the concentration data using the natural log function which serves to place the data on the same scale. The results of the log transformation are shown in Table 9 and FIGS. 29-32.

In summary, the results of this test demonstrated that this platform is (1) sufficiently reproducible for commercial purposes, (2) capable of detecting all 16 markers in most human plasma samples, and (3) able to discriminate between AD and NDC.

TABLE 7
SearchLight Cytokine Concentrations

| | | pg/ml | pg/ml | pg/ml | pg/ml | pg/ml | pg/ml | pg/ml | pg/ml | pg/ml |
|---|---|---|---|---|---|---|---|---|---|---|
| Test ID | Sample ID | hGCSF | hGDNF | hIL1a | hIL3 | hIL11 | hMCP3 | hIL8 | hEGF | hMIP1d |
| 1 | RC-001 | 40.0 | 271.3 | 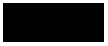 | 11.7 | 37.6 | 3.5 | 9.4 | 4.6 | 8349.8 |
| 2 | PP-001 | 44.3 | 250.5 | 0.8 | 14.2 | 34.6 | 2.2 | 10.8 | 5.3 | 11108.0 |
| 3 | RAAA818-00 | 3.4 | 67.9 | 0.8 | 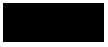 | 43.8 | 1.6 | 11.4 | 37.4 | 6059.0 |
| 4 | RAAA816-00 | 9.9 | 27.4 | 0.8 | 14.3 | 2.0 | 1.6 | 12.0 | 9.5 | 9275.4 |
| 5 | Pad-001 | 129.9 | 2453.3 | 24.6 | 195.4 | 57.0 | 3.8 | 11.8 | 10.8 | 9163.6 |
| 7 | Pad-001 | 73.7 | 2188.4 | 23.7 | 173.6 | 32.5 | 3.7 | 13.5 | 10.0 | 8367.3 |
| 6 | pNDC-001 | 110.5 | 231.2 | 7.4 | 45.1 | 93.1 | 1.8 | 21.5 | 10.7 | 11213.7 |
| 8 | pNDC-001 | 101.8 | 231.4 | 6.3 | 39.1 | 84.3 | 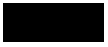 | 23.2 | 10.9 | 10379.0 |

| | | pg/ml | pg/ml | pg/ml | pg/ml | pg/ml | pg/ml | pg/ml |
|---|---|---|---|---|---|---|---|---|
| Test ID | Sample ID | hTNFa | hANG2 | hMCSF | hPDGFBB | hICAM1 | hRANTES | hPARC |
| 1 | RC-001 | 4.7 | 638.3 | 874.1 | 14.5 | 487638.9 | 35554.5 | 192148.2 |
| 2 | PP-001 | 4.7 | 507.4 | 768.0 | 17.2 | 370698.1 | 18836.4 | 110395.0 |
| 3 | RAAA818-00 | 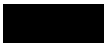 | 483.9 | 712.7 | 40.4 | 438750.5 | 293759.5 | 119660.9 |
| 4 | RAAA816-00 | 4.7 | 571.7 | 536.7 | 37.1 | 524744.9 | 19785.5 | 142833.9 |
| 5 | Pad-001 | 35.7 | 605.6 | 350.0 | 146.3 | 405431.6 | 114826.0 | 180866.4 |
| 7 | Pad-001 | 32.3 | 531.9 | 384.1 | 122.4 | 378719.7 | 98466.8 | 166373.2 |
| 6 | pNDC-001 | 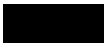 | 766.8 | 742.2 | 28.5 | 457380.4 | 21022.9 | 134185.4 |
| 8 | pNDC-001 | 6.1 | 705.9 | 655.4 | 17.5 | 401449.6 | 16938.5 | 123660.9 |

TABLE 8
Searchlight Within Run Sample Correlations
Rsquared values

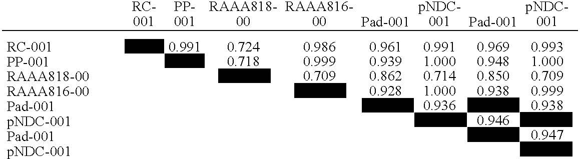

TABLE 9
Natural Log-transformed SearchLight Cytokine Concentrations

| | | Ln-transformed | | | | | | | |
|---|---|---|---|---|---|---|---|---|---|
| Test | | hGCSF | hGDNF | hIL1a | hIL3 | hIL11 | hMCP3 | hIL8 | hEGF |
| ID | Sample ID | pg/ml | pg/ml | pg/ml | pg/ml | pg/ml | pg/ml | pg/ml | pg/ml |
| 1 | RC-001 | 3.6888 | 5.6033 | −0.29 | 2.458 | 3.628 | 1.255 | 2.238 | 1.529 |
| 2 | PP-001 | 3.7917 | 5.5236 | −0.22 | 2.65 | 3.544 | 0.7858 | 2.379 | 1.666 |
| 3 | RAAA818-00 | 1.2144 | 4.2177 | −0.22 | 1.741 | 3.779 | 0.47 | 2.431 | 3.621 |
| 4 | RAAA816-00 | 2.2946 | 3.3092 | −0.22 | 2.658 | 0.693 | 0.47 | 2.488 | 2.256 |
| 5 | Pad-001 | 4.8671 | 7.8052 | 3.201 | 5.275 | 4.044 | 1.3404 | 2.465 | 2.384 |
| 6 | pNDC-001 | 4.7053 | 5.4435 | 1.997 | 3.809 | 4.533 | 0.6148 | 3.066 | 2.375 |
| 7 | Pad-001 | 4.2999 | 7.6909 | 3.165 | 5.157 | 3.48 | 1.3149 | 2.604 | 2.305 |
| 8 | pNDC-001 | 4.6234 | 5.4444 | 1.838 | 3.665 | 4.434 | 0.4057 | 3.145 | 2.388 |

| | Ln-transformed | | | | | | | |
|---|---|---|---|---|---|---|---|---|
| Test | hMIP1d | hTNFa | hANG2 | hMCSF | hPDGFBB | hICAM1 | hRANTES | hPARC |
| ID | pg/ml | pg/ml | pg/ml | pg/ml | pg/ml | pg/ml | pg/ml | pg/ml |
| 1 | 9.03 | 1.5476 | 6.4589 | 6.7732 | 2.6753588 | 13.0973 | 10.47882 | 12.166 |
| 2 | 9.3154 | 1.5476 | 6.2293 | 6.6438 | 2.8464151 | 12.8231 | 9.843549 | 11.6118 |
| 3 | 8.7093 | 0.261 | 6.1819 | 6.5691 | 3.6984158 | 12.9917 | 12.59052 | 11.6924 |
| 4 | 9.1351 | 1.5476 | 6.3487 | 6.2853 | 3.6144608 | 13.1707 | 9.892705 | 11.8694 |
| 5 | 9.123 | 3.5744 | 6.4062 | 5.8579 | 4.9854206 | 12.9127 | 11.65117 | 12.1055 |
| 6 | 9.3249 | 0.8839 | 6.6423 | 6.6096 | 3.3500528 | 13.0333 | 9.953369 | 11.807 |
| 7 | 9.0321 | 3.4763 | 6.2765 | 5.9509 | 4.8075311 | 12.8446 | 11.49747 | 12.022 |
| 8 | 9.2475 | 1.8017 | 6.5594 | 6.4853 | 2.8636414 | 12.9028 | 9.737341 | 11.7253 |

### Examples 3-11

The following Examples 3-11 were published in U.S. patent application Ser. Nos. 11/148,595, filed Jun. 8, 2005, and 11/580,405, filed Oct. 13, 2006, both of which are incorporated by reference herein in their entireties.

### Example 3

### AD Diagnosis Biomarkers

We compared plasma protein expression levels for 120 proteins in 32 cases of serum collected from patients with Alzheimer's Disease (with a mean age of 74) to 19 cases of serum collected from control subjects (also with mean age of 74). Alzheimer's Disease subjects were clinically diagnosed with AD by a neurologist, and had Mini Mental State Exam (MMSE) scores ranging from 26-14.

Plasma samples were assayed using a sandwich-format ELISA on a nitrocellulose filter substrate. Plasma samples were diluted 1:10 in phosphate buffer and incubated with the capture substrate (a nitrocellulose membrane spotted with capture antibodies). The samples were incubated with the capture substrate for two hours at room temperature, then decanted from the capture substrate. The substrate was washed twice with 2 ml of washing buffer (1×PBS; 0.05% Tween-20) at room temp, then incubated with biotinylated detection antibodies for two hours at room temperature. The capture antibody solution was decanted and the substrate was washed twice for 5 min with washing buffer. The washed substrate was then incubated with horseradish peroxidase/streptavidin conjugate for 45 minutes, at which time the conjugate solution was decanted and the membranes were washed with washing buffer twice for 5 minutes. The substrate was transferred onto a piece of filter paper, incubated in enhanced chemiluminescence (ECL) Detection Buffer solution purchased from Raybiotech, Inc. Chemiluminescence was detected and quantified with a chemiluminescence imaging camera. Signal intensities were normalized to standard proteins blotted on the substrate and used to calculate relative levels of biomarkers. In other examples, signal intensities were normalized to the median and used to calculate relative levels of biomarkers. Measured levels of any individual biomarkers can be normalized by comparing the level to the mean or median measured level of two or more biomarkers from the same individual.

Relative biomarker levels in plasma are compared between control and AD groups revealing 46 discriminatory biomarkers: GCSF; IFN-g; IGFBP-1; BMP-6; BMP-4; Eotaxin-2; IGFBP-2; TARC; RANTES; ANG; PARC; Acrp30; AgRP(ART); TIMP-1; TIMP-2; ICAM-1; TRAIL R3; uPAR; IGFBP-4; LEPTIN(OB); PDGF-BB; EGF; BDNF; NT-3; NAP-2; IL-1ra; MSP-a; SCF; TGF-b3; TNF-b MIP-1d; IL-3; FGF-6; IL-6 R; sTNF RII; AXL; bFGF; FGF-4; CNTF; MCP-1; MIP-1b; TPO; VEGF-B; IL-8; FAS; EGF-R. An unsupervised clustering (that is, the clustering algorithm does not know which cases are AD and which are normal) of the 46 discriminatory markers results in the clustering of the samples into 2 groups or clusters, a cluster of control samples, and a cluster of AD samples. Sensitivity was calculated as the number of correctly classed AD samples in the AD cluster/total number of AD samples, which is 29/32 or 90.6%. Specificity was calculated as total number of correctly classed control samples in the control cluster/total number of controls, which is (14/19=73.6%).

Biomarker levels were compared between control and AD groups, revealing 20 biomarkers (shown in Table 10) that are differentially regulated (each is decreased in AD as compared to control) between the two groups. Statistical analysis was performed to find the probability that the finding of differential levels was in error (the “q” value) for any one biomarker. Biomarkers with differential levels and associated q values (shown as percentage values) are shown in Table 10 (fold change indicates the fold change between levels in control vs. AD samples). Sensitivity was calculated as number of AD samples in AD cluster/total number of AD samples, which is 29/32 or 90.6%. Specificity was calculated as total correctly predicted AD/total predicted AD (29/34=85%).

TABLE 10

| | Fold | |
|---|---|---|
| | Change | |
| | (as negative | |
| | value or | q-value |
| Qualitative Biomarker | decrease) | (%) |
| Brain derived neurotrophic factor (BDNF) | 0.536 | 1.656 |
| Basic fibroblast growth factor (bFGF) | 0.673 | 1.656 |
| Epidermal growth factor (EGF) | 0.561 | 1.656 |
| Fibroblast growth factor-6 (FGF-6) | 0.664 | 1.656 |
| Interleukin-3 (IL-3) | 0.758 | 1.656 |
| Soluble interleukin-6 receptor (sIL-6 R) | 0.676 | 1.656 |
| Leptin (also known as OB) | 0.476 | 1.656 |
| Macrophage inflammatory protein 1-delta | 0.542 | 1.656 |
| (MIP-1δ) | | |
| MSP-a | 0.764 | 1.656 |
| NAP-2 | 0.672 | 1.656 |
| Neurotrophin-3 (NT-3) | 0.698 | 1.656 |
| Platelet derived growth factor, BB dimer | 0.536 | 1.656 |
| (PDGF-BB) | | |
| RANTES | 0.682 | 1.656 |
| Stem cell factor (SCF) | 0.730 | 1.656 |
| sTNF RII | 0.700 | 1.656 |
| Transforming growth factor beta-3 (TGF-β3) | 0.769 | 1.656 |
| Tissue inhibitor of metalloproteases-1 (TIMP-1) | 0.716 | 1.656 |
| Tissue inhibitor of metalloproteases-2 (TIMP-2) | 0.750 | 1.656 |
| Tumor necrosis factor beta (TNF-β) | 0.649 | 1.656 |
| TPO | 0.714 | 1.656 |

### Example 4

### Decision Trees from AD Diagnosis Marker Data

Upon further analysis of the data from Example 3, two different decision trees were formulated for diagnosis of AD using AD diagnosis biomarkers.

The first decision tree utilizes sIL-6R, IL-8, and TIMP-1 levels. The rules which make up the decision tree are: (1) If sIL-6R≦5.18 and IL-8 is ≦0.957, the indication is normal; (2) if sIL-6R≦5.18 and IL-8>0.957, the indication is AD; (3) if sIL-6R>5.18 and TIMP-1≦7.978, the indication is AD; and (4) if sIL-6R>5.18 and TIMP-1 is >7.978, the indication is normal, wherein the values expressed are relative concentrations.

Accuracy of this decision tree was measured using 10-fold cross-validation testing feature in CART to generate misclassification rates for learning samples and testing samples. Sensitivity was calculated from the testing scores as number of AD samples correctly predicted as AD/total number of AD samples (29/32=0.906). Specificity was calculated from the testing scores as total correctly predicted cases of AD/total number of cases predicted AD (29/33=0.878).

A second decision tree was formulating using BDNF, TIMP-1 and MIP-1δ levels. The rules which make up the decision tree are: (1) if BDNF>4.476, the indication is normal; (2) if BDNF≦4.476 and TIMP-1≦8.942, the indication is AD; (3) if BDNF≦4.476, TIMP-1>8.942, and MIP-1δ≦1.89, the indication is AD; and (4) if BDNF<4.476, TIMP-1>8.942, and MIP-1δ>1.89, the indication is normal. Accuracy of this decision tree was measured using 10-fold cross-validation testing feature in CART to generate misclassification rates for learning samples and testing samples. Sensitivity was calculated from the testing scores as number of AD samples correctly predicted as AD/total number of AD samples (0.875). Specificity was calculated from the testing scores as total correctly predicted cases of AD/total number of cases predicted AD (0.82).

### Example 5

### Diagnosis of MCI

Levels of RANTES and Leptin were measured in 18 samples from control subjects (mean age=74) and 6 samples from patients diagnosed with mild cognitive impairment (MCI). MCI patients had been clinically diagnosed by a neurologist, and had an AULT-A7 score of less than 5 and Mini Mental State Exam (MMSE) scores ranging from 30-28. Control subjects had an AULT-A7 score greater than or equal to 5 and MMSE score ranging from 30-28.

RANTES and Leptin levels were measured using an ELISA kit from R&D systems according to the manufacturer's instructions. The raw ELISA expressions values were normalized by dividing each value by the median of all the samples. Analysis of the data showed (a) Leptin is not decreased in MCI patients as compared to control subjects (in the six MCI samples, Leptin was actually 11% higher than the control subjects), and (b) a bimodal distribution of RANTES, where MCI patients had RANTES levels of between 1.043 and 1.183 (levels from control subjects were either ≦1.043 or >1.183). However, closer inspection of the data led us to believe that those control subjects with RANTES≦1.043 had been incorrectly classified as normal (and should have been diagnosed as MCI).

Reclassification of control subjects with RANTES≦1.043 as MCI patients allows the creation of a simple rule: if RANTES≦1.183 and Leptin >=0.676, the indication is MCI. Sensitivity and specificity, calculated as described in Example 4, were 83.3% and 88.88%, respectively.

### Example 6

### Monitoring and Stratification of AD Patients

Levels of RANTES, Leptin, PDGF-BB, and BDNF were measured in serum samples collected from 36 patients diagnosed with Alzheimer's Disease. (mean age of 74) using ELISA kits from R&D systems according to the manufacturer's instructions. The raw ELISA expressions values were normalized by dividing each value by the median of all the samples. The samples were grouped into three classes on the basis of MMSE score: Class 1 (mild AD), MMSE 27-22; Class 2 (moderate AD), MMSE 21-16; and Class 3 (severe AD), MMSE 15-12.

Upon analysis of the ELISA data, we formulated a decision tree using BDNF and PDGF-BB. The rules which make up the decision tree are: (1) if BDNF≦0.626, the indication is mild AD; (2) if BDNF>0.626 and PDGF-BB≦0.919, the indication is moderate AD; and (3) if BDNF>0.626 and PDGF-BB>0.919, the indication is severe AD. The values expressed are relative concentrations that have been normalized to the median. Average normalized levels for Leptin were: Class I=0.886; class II=0.757; class III=0.589. Average normalized levels for BDNF were: Class I=0.595; class II=0.956; class III=1.23. When applied to a set of “test” data, the decision tree produced 58%, 47%, and 57% percent correct stratification of the test samples into mild, moderate, and severe categories.

### Example 7

### Four Discriminatory Markers

The absolute concentrations in plasma of only 4 discriminatory markers, BDNF, PDGF-BB, LEPTIN, and RANTES measured by ELISA was used to classify samples. ELISA kits were purchased from R&D Systems, and measurements were obtained according to manufacturer recommendations. For example for RANTES, the following protocol was followed.

1. Add 50 μL standards, specimens or controls to appropriate wells.

2. Add 50 μL anti-RANTES Biotin Conjugate to each well.

3. Incubate wells at 37° C. for 1 hour.

4. Aspirate and wash wells 4× with Working Wash Buffer.

5. Add 100 μL Streptavidin-HRP Working Conjugate to each well.

6. Incubate for 30 minutes at room temperature.

7. Aspirate and wash wells 4× with Working Wash Buffer.

8. Add 100 μL of Stabilized Chromogen to each well.

9. Incubate at room temperature for 30 minutes in the dark.

10. Add 100 μL of Stop Solution to each well.

11. Read absorbance at 450 nm.

Following the above protocol, an unsupervised clustering of BDNF, PDGF-BB, LEPTIN, and RANTES was performed using the publicly available web based clustering software wCLUTO at cluto.ccgb.umn.edu/cgi-bin/wCluto/wCluto.cgi. Here the clustering of the 4 proteins resulted in the clustering of the samples into 2 groups or clusters, a cluster of control samples and a cluster of AD samples. Sensitivity was calculated as the number of correctly classed AD samples in the AD cluster/total number of AD samples, which is 21/24 or 87.5%. Specificity was calculated as total number of correctly classed control samples in the control cluster/total number of controls, which is 20/24=83.3%.

Additionally, absolute biomarker levels in plasma (as measured by ELISA) for BDNF, PDGF-BB, and LEPTIN, were correlated with MMSE scores (range 12-30). AD could be identified in MMSE scores in a range of 12-28 and control samples were identified in MMSE scores in the range of 25-30. Table 11 shows the correlations and their statistical significance (p-value). The upper and lower correlations show whether the upper end of the range of MMSE scores and biomarker concentrations or the lower end of the range of MMSE scores and biomarker concentrations are more correlated. Therefore, the correlations show that higher levels of BDNF and Leptin are significantly correlated with better MMSE scores, and that increase in the concentration of BDNF and Leptin from a reference point or an earlier collection is an indication of improvement in cognition as measured by MMSE. Simultaneously, or by itself, the lower the levels of PDGF-BB in men is significantly correlated with better MMSE scores, and a decrease in the concentration of PDGF-BB in male sample compared to an earlier collection in that male, is an indication of improvement in cognition as measured by MMSE.

The results show (Table 11) the correlation between the plasma concentration of 3 discriminatory proteins for AD to the MMSE score of the subjects and the correlation between concentrations of proteins that are discriminatory for AD. There was no correlation between MMSE score and Age among AD subjects and there was no correlation between Age and the concentration of BDNF, PDGF-BB, or LEPTIN in plasma among AD subjects. The p-values show that the correlations are statistically significant. The count shows the number of cases. BDNF has a statistically significant positive correlation with MMSE scores. PDGF-BB has a statistically significant negative correlation with MMSE scores in men. LEPTIN has a statistically significant positive correlation with MMSE scores. This experiment demonstrates that plasma concentrations for PDGF-BB, LEPTIN, and BDNF can be used to monitor the progression of cognitive decline.

TABLE 11

| | | | | | 95% | 95% |
|---|---|---|---|---|---|---|
| | Correlation | Count | Z-value | P-value | Lower | Upper |
| BDNF to MMSE | 0.184 | 165 | 2.373 | 0.0176 | 0.032 | 0.328 |
| BDNF to MMSE (Females) | 0.229 | 91 | 2.18 | 0.0289 | 0.024 | 0.415 |
| PDGF-BB to MMSE (Males) | −0.207 | 74 | −1.769 | 0.0768 | −0.416 | 0.023 |
| LEPTIN to MMSE | 0.193 | 164 | 2.478 | 0.0132 | 0.041 | 0.336 |
| BDNF to PDGF-BB | 0.700 | 181 | 11.575 | 0.0001 | 0.617 | 0.768 |
| PDGF-BB to RANTES | 0.563 | 181 | 8.5 | 0.0001 | 0.454 | 0.655 |
| BDNF to RANTES | 0.714 | 181 | 11.9 | 0.0001 | 0.634 | 0.779 |

Controls and AD cases were age matched, and had a mean age of 74. The mean MMSE score for AD cases (n=24) was 20, while the mean MMSE score for Control cases (n=24) was 30. Classification of the samples was performed with unsupervised clustering of protein concentration. The total accuracy of classification was 85.4%. This results demonstrated that plasma protein concentrations for BDNF, PDGF-BB, LEPTIN, and RANTES, as measured by ELISA can be used to accurately discriminate between AD and controls.

### Example 8

### Validation of Mean Protein Concentrations in AD and Controls by ELISA

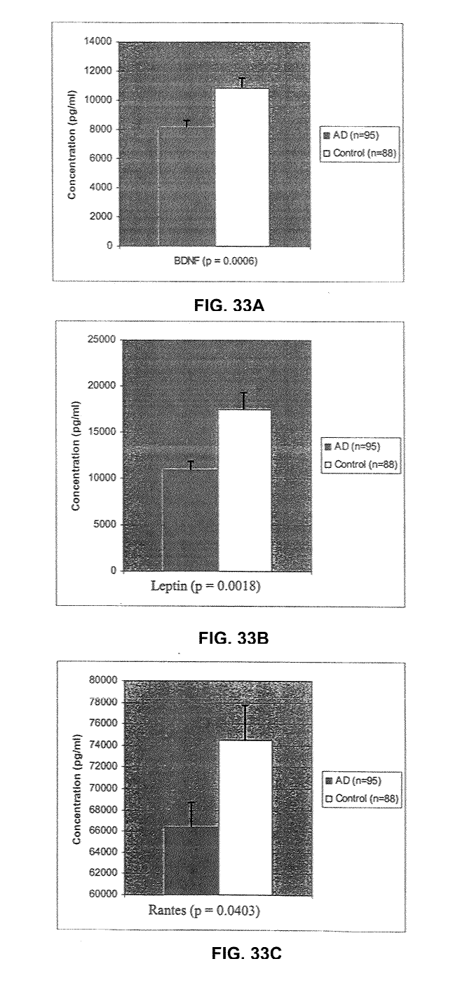

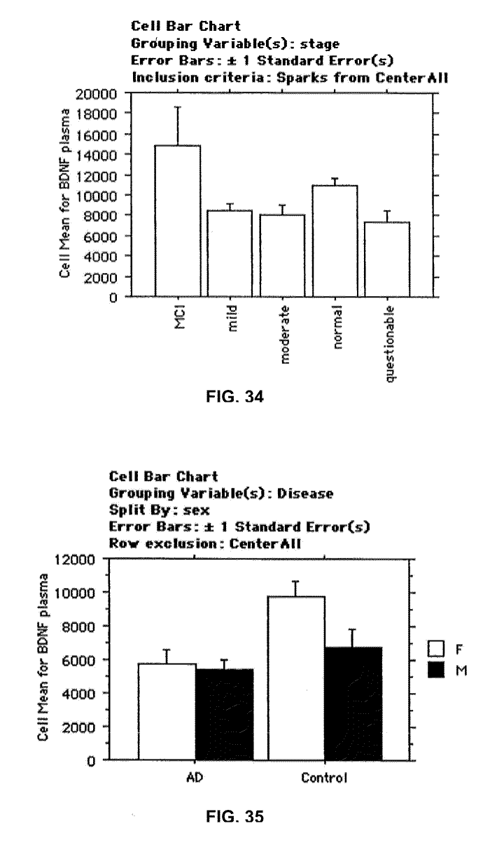

Protein concentrations for proteins, LEPTIN, BDNF and RANTES, in plasma samples of AD (n=95) to age matched Controls (n=88) are shown in FIGS. 33A-33C. One of the four proteins we measured was Brain Derived Neurotrophic Factor (BDNF). The mean concentration of BDNF in AD plasma was 8.1 ng/ml (SE+/−0.4) compared to the mean of control plasma 10.8 ng/ml (SE+/−0.68) and the difference was found to be extremely statistically significant (p-value=0.0006). We also found that the concentrations of BDNF were lower in other forms of dementia (5.74 ng/ml, n=20) than AD. The mean concentration of a second protein Leptin in AD plasma was found to be 10.9 ng/ml (SE+/−1.06) compared to the mean of control plasma 17.4 ng/ml (SE+/−1.8) and the difference was found to be statistically very significant (p-value=0.0018). The mean concentration of a third protein RANTES in AD plasma was found to be 66.3 ng/ml (SE+/−2.4) compared to control samples 74.5 ng/ml (SE+/−3.2) and the difference was found to be statistically significant (p-value=0.0403). No difference in the means of concentrations for RANTES, PDGF-BB, and BDNF were observed among AD subjects with MMSE scores=/>20 (n=54) and those <20 (n=41).

### Example 9

### Absolute Biomarker Concentrations in Plasma

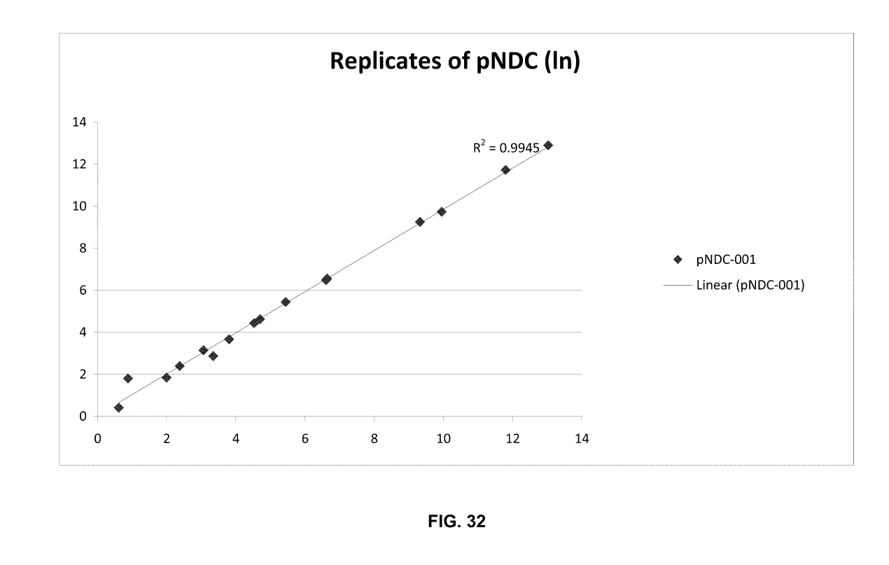

Additionally, absolute biomarker concentrations in plasma were measured for BDNF, and mean concentrations for Controls was compared to MCI (Mild Cognitive Impairment), MMSE 25-28, MMSE 20-25, and MMSE 10-20. For the purposes of this experiment, the index used in the following example is: questionable AD is =MMSE score in the range of 25-28; mild AD=MMSE score in the range of 20-25; and moderate AD=MMSE score in the range of 10-20 and severe AD=MMSE score in the range of 10-20. For the purpose of Example 9, all individuals assessed as having Questionable AD were diagnosed by a physician as having AD. The FIG. 32 shows that mean concentrations of BDNF in plasma for MMSE 25-28; MMSE 20-25; MMSE 10-20 are significantly lower than the mean concentration in Controls (Normal, mean age 74) and the mean concentration of BDNF in MCI is significantly higher than in Controls and all cases of AD. FIG. 32.
Unpaired t-test for BDHF plasma
Grouping Variable: stage
Hypothesized Difference=0
Inclusion criteria: Sparks from Center All

| | Mean Diff. | DF | t-Value | P-Value |
|---|---|---|---|---|
| MCI, mild | 6349.252 | 47 | 3.050 | .0038 |
| MCI, moderate | 6828.574 | 31 | 2.651 | .0125 |
| MCI, normal | 3961.358 | 86 | 1.442 | .1529 |
| MCI, questionable | 7547.218 | 17 | 2.550 | .0207 |
| mild, moderate | 479.322 | 68 | .460 | .6467 |
| mild, normal | −2387.894 | 123 | −2.270 | .0250 |
| mild, questionable | 1197.966 | 54 | .969 | .3369 |
| moderate, normal | −2867.216 | 107 | −2.175 | .0319 |
| moderate, questionable | 718.644 | 38 | .475 | .6372 |
| normal, questionable | 3585.860 | 93 | 1.993 | .0492 |

Group Info for BDNF plasma
Grouping Variable: stage
Inclusion criteria: Sparks from Center All

| | Count | Mean | Variance | Std. Dev. | Std. Err |
|---|---|---|---|---|---|
| MCI | 6 | 14879.833 | 85932530.967 | 9269.980 | 3784.454 |
| mild | 43 | 8530.581 | 15299257.963 | 3911.427 | 596.487 |
| moderate | 27 | 8051.259 | 22317487.815 | 4724.139 | 909.161 |
| normal | 82 | 10918.476 | 39478328.993 | 6283.178 | 693.861 |
| question- | 13 | 7332.615 | 15122872.923 | 3888.814 | 1078.563 |
| able | | | | | |

Additionally, absolute concentrations of BDNF, in plasma samples collected from four separate Alzheimer's Centers was compared for gender differences in mean concentrations between AD (Females) and Control (Females) and AD (Males) and Control (Males). FIG. 35 shows that there is 40% difference in the concentration of BDNF in AD Females compared to Control Females and the difference is highly statistically significant (p-value=0.004). The difference in the mean concentration of BDNF for all AD cases compared to all Control case was found to be extremely statistically significant (p-value=0.0006).
Unpaired t-test for BDHF plasma
Grouping Variable Disease
Split By: sex
Hypothesized Difference=0
Row exclusion: Center All

| | Mean Diff. | DF | t-Value | P-Value |
|---|---|---|---|---|
| AD, Control: Total | −2974.140 | 187 | −3.482 | .0006 |
| AD, Control: F | −3939.353 | 87 | −2.924 | .0044 |
| AD, Control: M | −1348.601 | 92 | −1.165 | .2469 |

Results for totals may not agree with results for individual cells because of missing values for split variables.
Group Info for BDHF plasma
Grouping Variable: Disease
Split By: sex
Row exclusion: Center All

| | Count | Mean | Variance | Std. Dev. | Std. Err |
|---|---|---|---|---|---|
| AD: Total | 106 | 5596.113 | 24323422.844 | 4931.878 | 479.026 |
| AD: F | 38 | 5775.921 | 25121499.318 | 5012.135 | 813.076 |
| AD: M | 62 | 5396.774 | 24336564.079 | 4933.210 | 626.518 |
| Control: | 83 | 8570.253 | 46322420.606 | 6806.058 | 747.062 |
| Total | | | | | |
| Control: F | 51 | 9715.275 | 50173107.603 | 7083.298 | 991.860 |
| Control: | 32 | 6745.375 | 36011373.274 | 6000.948 | 1060.828 |
| M | | | | | |

Results for totals may not agree with results for individual cells because of missing values for split variables.

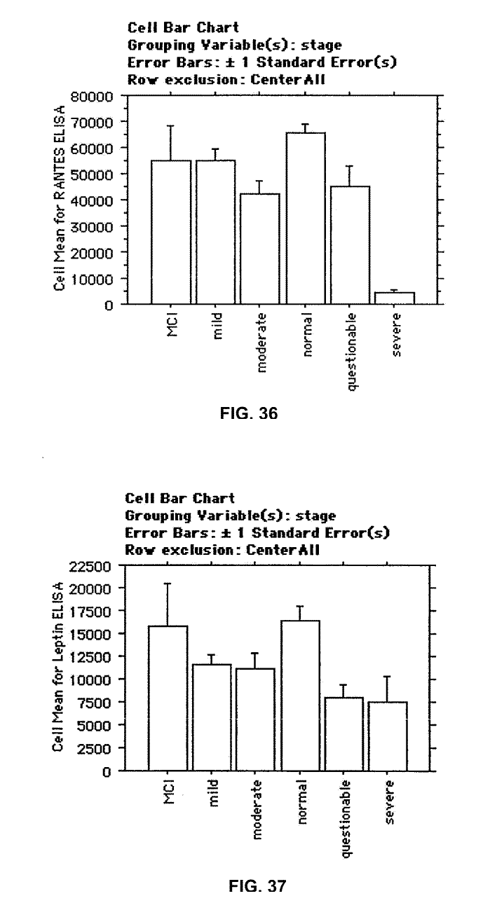

Additionally, absolute biomarker concentrations in plasma were measured for RANTES in plasma samples collected from four different Alzheimer's Centers, and mean concentrations for Controls were compared to MCI (Mild Cognitive Impairment), MMSE 25-28; (MMSE 20-25; MMSE 10-20; and MMSE 10-20. The index is described above. The mean differences between Mild AD compared to Moderate AD, Mild AD compared to Normal, Mild AD compared to Severe AD, Moderate AD compared to Normal, Questionable AD compared to Normal, Normal to Severe AD were all found to be statistically significant. FIG. 36.
Unpaired t-test for RANTES ELISA
Grouping Variable: stage
Hypothesized Difference=0
Row exclusion: Center All

| | Mean Diff. | DF | t-Value | P-Value |
|---|---|---|---|---|
| MCI, mild | 84.789 | 64 | .007 | .9945 |
| MCI, moderate | 12454.688 | 51 | 1.042 | .3022 |
| MCI, normal | −10422.892 | 106 | −.866 | .3884 |
| MCI, questionable | 9682.438 | 29 | .682 | .5007 |
| MCI, severe | 50349.200 | 10 | 1.647 | .1305 |
| mild, moderate | 12369.899 | 97 | 1.814 | .0728 |
| mild, normal | −10507.681 | 152 | −1.775 | .0780 |
| mild, questionable | 9597.649 | 75 | 1.081 | .2830 |
| mild, severe | 50264.411 | 56 | 2.031 | .0470 |
| moderate, normal | −22877.580 | 139 | −3.606 | .0004 |
| moderate, questionable | −2772.250 | 62 | −.315 | .7535 |
| moderate, severe | 37894.512 | 43 | 1.647 | .1069 |
| normal, questionable | 20105.330 | 117 | 2.353 | .0203 |
| normal, severe | 60772.092 | 98 | 2.395 | .0185 |
| questionable, severe | 40666.762 | 21 | 1.624 | .1192 |

Group Info for RANTES ELISA

Grouping Variable: stage

Row exclusion: Center All

| | Count | Mean | Variance | Std. Dev. | Std. Err |
|---|---|---|---|---|---|
| MCI | 10 | 54919.200 | 1729660285.733 | 41589.185 | 13151.655 |
| mild | 56 | 54834.411 | 1203622609.701 | 34693.265 | 4636.082 |
| moder- | 43 | 42464.512 | 1036226732.256 | 32190.476 | 4909.002 |
| ate | | | | | |
| normal | 98 | 65342.092 | 1275358885.672 | 35712.167 | 3607.474 |
| ques- | 21 | 45236.762 | 1201710117.890 | 34665.691 | 7564.674 |
| tion- | | | | | |
| able | | | | | |
| severe | 2 | 4570.000 | 2976800.000 | 1725.341 | 1220.000 |

Additionally, absolute biomarker concentrations in plasma were measured for Leptin in plasma samples collected from four different Alzheimer's Centers, and mean concentrations for Controls were compared to MCI (Mild Cognitive Impairment); MMSE 25-28; MMSE 20-25; MMSE 10-20; and MMSE 10-20. The mean differences between Questionable AD compared to MCI, Mild AD compared to Normal, Mild AD compared to Questionable AD, Questionable AD compared to Normal, and Moderate AD compared to Normal were all found to be statistically significant. FIG. 37.
Unpaired t-test for Leptin ELISA
Grouping Variable: stage
Hypothesized Difference=0
Row exclusion: Center All

| | Mean Diff. | DF | t-Value | P-Value |
|---|---|---|---|---|
| MCI, mild | 4164.889 | 64 | 1.338 | .1856 |
| MCI, moderate | 4707.044 | 51 | 1.061 | .2939 |
| MCI, normal | −650.092 | 105 | −.123 | .9022 |
| MCI, questionable | 7793.348 | 29 | 2.000 | .0550 |
| MCI, severe | 8187.800 | 10 | .739 | .4767 |
| mild, moderate | 542.155 | 97 | .272 | .7860 |
| mild, normal | −4814.981 | 151 | −2.117 | .0359 |
| mild, questionable | 3628.458 | 75 | 1.897 | .0617 |
| mild, severe | 4022.911 | 56 | .734 | .4661 |
| moderate, normal | −5357.136 | 138 | −1.963 | .0516 |
| moderate, questionable | 3086.303 | 62 | 1.085 | .2822 |
| moderate, severe | 3480.756 | 43 | .403 | .6892 |
| normal, questionable | 8443.439 | 116 | 2.368 | .0195 |
| normal, severe | 8837.892 | 97 | .778 | .4383 |
| questionable, severe | 394.452 | 21 | .078 | .9383 |

Group Info for Leptin ELISA
Grouping Variable: stage
Row exclusion: Center All

| | Count | Mean | Variance | Std. Dev. | Std. Err |
|---|---|---|---|---|---|
| MCI | 10 | 15727.300 | 225300738.678 | 15010.021 | 4746.585 |
| mild | 56 | 11562.411 | 58790550.756 | 7667.500 | 1024.613 |
| moderate | 43 | 11020.256 | 145797834.909 | 12074.677 | 1841.371 |
| normal | 97 | 16377.392 | 255125297.032 | 15972.642 | 1621.776 |
| question- | 21 | 7933.952 | 47833192.348 | 6916.154 | 1509.229 |
| able | | | | | |
| severe | 2 | 7539.500 | 16125520.500 | 4015.659 | 2839.500 |

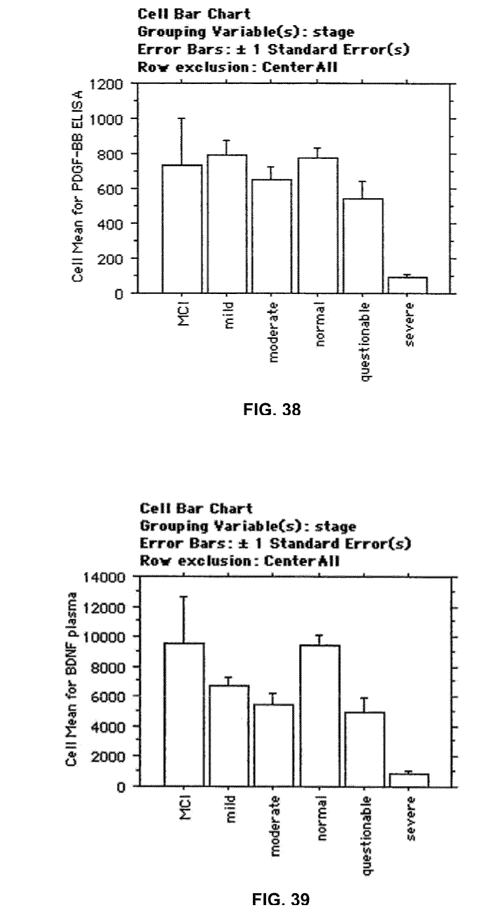

Additionally, absolute biomarker concentrations in plasma were measured for PDGF-BB in plasma samples collected from four different Alzheimer's Centers, and mean concentrations for Controls were compared to MCI (Mild Cognitive Impairment); MMSE 25-28; MMSE 20-25; MMSE 10-20; and MMSE 10-20. The mean differences between Questionable AD compared to Mild AD, Mild AD compared to Severe AD, Moderate AD compared to Severe AD, Normal compared to Questionable AD, and Normal to Severe AD were all found to be statistically significant. FIG. 38.
Unpaired t-test for PDGF-BB ELBA
Grouping Variable: stage
Hypothesized Difference=0
Row exclusion: Center All

| | Mean Diff. | DF | t-Value | P-Value |
|---|---|---|---|---|
| MCI, mild | −62.275 | 58 | −.286 | .7756 |
| MCI, moderate | 81.595 | 44 | .411 | .6831 |
| MCI, normal | −42.865 | 103 | −.210 | .8343 |
| MCI, questionable | 191.571 | 28 | .810 | .4246 |
| MCI, severe | 637.000 | 9 | 1.072 | .3117 |
| mild, moderate | 143.869 | 86 | 1.285 | .2023 |
| mild, normal | 19.410 | 145 | .199 | .8426 |
| mild, questionable | 253.846 | 70 | 1.812 | .0742 |
| mild, severe | 699.275 | 51 | 1.745 | .0871 |
| moderate, normal | −124.459 | 131 | −1.201 | .2320 |
| moderate, questionable | 109.977 | 56 | .869 | .3885 |
| moderate, severe | 555.405 | 37 | 1.716 | .0945 |
| normal, questionable | 234.436 | 115 | 1.767 | .0799 |
| normal, severe | 679.865 | 96 | 1.696 | .0931 |
| questionable, severe | 445.429 | 21 | 1.278 | .2153 |

Group Info for PDGF-BB ELISA
Grouping Variable: stage
Row exclusion: Center All

| | Count | Mean | Variance | Std. Dev. | Std. Err |
|---|---|---|---|---|---|
| MCI | 9 | 731.000 | 650139.000 | 806.312 | 268.771 |
| mild | 51 | 793.275 | 315391.883 | 561.598 | 78.639 |
| moderate | 37 | 649.405 | 204231.470 | 451.920 | 74.295 |
| normal | 96 | 773.865 | 318171.171 | 564.067 | 57.570 |
| questionable | 21 | 539.429 | 233024.657 | 482.726 | 105.340 |
| severe | 2 | 94.000 | 648.000 | 25.456 | 18.000 |

Additionally, absolute biomarker concentrations in plasma were measured for BDNF in plasma samples collected from four different Alzheimer's centers, and means concentrations for Controls were compared to MCI (Mild Cognitive Impairment), Questionable AD (MMSE 25-28), Mild differences between MCI compared to Moderate AD, MCI compared to Questionable AS, Mild AD to Normal, Mild AD to sever AD, Moderate to Normal, Normal to Questionable AD, and Normal to Severe were all found to be statistically significant. FIG. 39.
Unpaired t-test for BDNF plasma
Grouping Variable: stage
Hypothesized Difference=0
Row exclusion: Center All

| | Mean Diff. | DF | t-Value | P-Value |
|---|---|---|---|---|
| MCI, mild | 2819.186 | 64 | 1.433 | .1568 |
| MCI, moderate | 4071.016 | 51 | 1.877 | .0663 |
| MCI, normal | 124.278 | 106 | .053 | .9578 |
| MCI, questionable | 4535.757 | 29 | 1.806 | .0813 |
| MCI, severe | 8660.400 | 10 | 1.202 | .2570 |
| mild, moderate | 1251.831 | 97 | 1.262 | .2098 |
| mild, normal | −2694.908 | 152 | −2.638 | .0092 |
| mild, questionable | 1716.571 | 75 | 1.447 | .1520 |
| mild, severe | 5841.214 | 56 | 1.726 | .0898 |
| moderate, normal | −3946.739 | 139 | −3.431 | .0008 |
| moderate, questionable | 464.741 | 62 | .360 | .7199 |
| moderate, severe | 4589.384 | 43 | 1.265 | .2128 |
| normal, questionable | 4411.480 | 117 | 2.868 | .0049 |
| normal, severe | 8536.122 | 98 | 1.781 | .0781 |
| questionable, severe | 4124.643 | 21 | 1.321 | .2006 |

Group Info for BDNF plasma
Grouping Variable: stage
Row exclusion: Center All

| | Count | Mean | Variance | Std. Dev. | Std. Err |
|---|---|---|---|---|---|
| MCI | 10 | 9511.900 | 96113654.322 | 9803.757 | 3100.220 |
| mild | 56 | 6692.714 | 22509096.208 | 4744.375 | 633.994 |
| moderate | 43 | 5440.884 | 25765123.534 | 5075.936 | 774.073 |
| normal | 98 | 9387.622 | 45504479.969 | 6745.701 | 681.419 |
| question- | 21 | 4976.143 | 18681976.129 | 4322.265 | 943.196 |
| able | | | | | |
| severe | 2 | 851.500 | 63724.500 | 252.437 | 178.500 |

It has been found that for Questionable AD (MMSE score in the range of 25-28) the levels of Leptin and PDGF-BB increase significantly whereas BDNF and RANTES do not change significantly. It has been found that from Mild AD (MMSE score in the range of 20-25) to Moderate AD (MMSE score in the range of 10-20) the level of LEPTIN does not decline whereas the levels for RANTES, BDNF and PDGF-BB declines.

### Example 10

In an attempt to identify proteins that are altered in the peripheral immune system in AD, expression levels of 120 cytokines, chemokines, and growth factors in plasma from 32 AD patients and 19 nondemented age-matched controls were measured using spotted antibody microarrays on filters. Statistical analysis identified 20 proteins as significantly different between AD and controls. Six of them including brain derived neurotrophic factor (BDNF) and NT-3, and PDGF-BB, EGF, FGF-6, bFGF, TGF-b3 have known neurotrophic activity and were significantly reduced in AD plasma. BDNF levels correlated with better cognitive function in the mini mental state exam (MMSE). BDNF measurements in plasma from two hundred AD cases and controls using commercial sandwich ELISA showed a highly significant 25% reduction in AD cases. Consistent with the array data, reduced plasma BDNF levels were associated with impaired memory function. BDNF is critical for neuronal maintenance, survival, and function. Without being bound by theory decreased blood levels of neurotrophins and BDNF may be linked with neurodegeneration and cognitive dysfunction in AD.

### Example 11

### Additional Biomarkers

Additionally, qualitative biomarker levels for GDNF, SDF-1, IGFBP3, FGF-6, TGF-b3, BMP-4, NT-3, EGF, BDNF, IGFBP-2 were correlated with MMSE scores (range 12-30) for AD (MMSE range 12-28) and control samples (MMSE range 25-30). Table 12 shows the correlations and their statistical significance (p-value). The upper and lower correlations show whether the upper end of the range of MMSE Scores and biomarker concentrations or the lower end of the range of MMSE scores and biomarker concentrations are more correlated. A negative correlation means that MMSE scores increase with decreasing levels of biomarker and vice versa. A positive correlation mean that MMSE scores increase with increasing levels of biomarker.

TABLE 12

| | | | | | 95% | 95% |
|---|---|---|---|---|---|---|
| | Correlation | Count | Z-value | P-value | Lower | Upper |
| GDNF to MMSE | −0.258 | 42 | −1.646 | 0.0997 | −0.521 | 0.05 |
| SDF-1 to MMSE | −0.363 | 42 | −2.375 | 0.0175 | −0.601 | −0.066 |
| IGFBP-3 to MMSE | 0.293 | 42 | 1.886 | 0.0593 | −0.012 | 0.548 |
| FGF-6 to MMSE | 0.471 | 42 | 3.192 | 0.0014 | 0.195 | 0.687 |
| TGF-b3 to MMSE | 0.317 | 42 | 2.049 | 0.0405 | 0.014 | 0.566 |
| BMP-4 to MMSE | 0.294 | 42 | 1.845 | 0.0583 | −0.011 | 0.545 |
| NT-3 to MMSE | 0.327 | 42 | 2.118 | 0.0342 | 0.025 | 0.574 |
| EGF to MMSE | 0.409 | 42 | 2.711 | 0.0067 | 0.12 | 0.634 |
| BDNF to MMSE | 0.464 | 42 | 3.139 | 0.0017 | 0.187 | 0.673 |
| IGFBP-2 to MMSE (Females) | 0.498 | 24 | 2.5 | 0.0123 | 0.118 | 0.75 |

### Example 12

This example shows Table 13, a Summary of Quantitative Markers for Identification and Stratification of AD.

TABLE 13

| | | Plasma | % Difference | |
|---|---|---|---|---|
| References | Samples | BioMarker | in Samples | p-value |
| Normal | Questionable AD | BDNF | −46% | 0.0049 |
| Normal | Questionable AD | Leptin | −52% | 0.0195 |
| Normal | Questionable AD | RANTES | −31% | 0.0203 |
| Normal | Questionable AD | PDGF-BB | −30% | 0.0799 |
| Normal | Mild AD | BDNF | −29% | 0.0092 |
| Normal | Mild AD | Leptin | −29% | 0.0359 |
| Normal | Mild AD | RANTES | −16% | 0.0780 |
| Normal | Moderate AD | BDNF | −42% | 0.0008 |
| Normal | Moderate AD | Leptin | −33% | 0.0359 |
| Normal | Moderate AD | RANTES | −35% | 0.0004 |
| Normal | Severe AD | BDNF | −90% | 0.0781 |
| Normal | Severe AD | RANTES | −93% | 0.0185 |
| Normal | Severe AD | PDGF-BB | −89% | 0.0931 |
| Questionable AD | Mild AD | Leptin | 45% | 0.0617 |
| Questionable AD | Mild AD | PDGF-BB | 46% | 0.0742 |
| Mild AD | Moderate AD | RANTES | −23% | 0.0780 |
| Mild AD | Severe AD | BDNF | −87% | 0.0898 |
| Mild AD | Severe AD | RANTES | −92% | 0.0470 |
| Mild AD | Severe AD | PDGF-BB | −88% | 0.0871 |
| Questionable AD | MCI | BDNF | 91% | 0.0813 |
| Questionable AD | MCI | Leptin | 98% | 0.0550 |
| MCI | Mild AD | BDNF | −42% | 0.0038 |

Accordingly, the present invention provides methods of aiding diagnosis of Alzheimer's disease (“AD”), comprising comparing a measured level of at least 4 AD diagnosis biomarkers, wherein said biomarkers comprise BDNF, PDGF-BB, Leptin and RANTES, in a biological fluid sample from an individual to a reference level for each AD diagnosis biomarker. Accordingly, methods are provided in which BDNF decreased at least about 10%, about 15%, about 20%, about 25% or about 30% as compared to a reference level of BDNF, indicates cognitive impairment, such as for example, an indication of AD. Accordingly, methods are provided in which Leptin decreased at least about 10%, about 15%, about 20%, about 25% or about 30% as compared to a reference level of Leptin, indicates cognitive impairment, such as for example, an indication of AD. Accordingly, methods are provided in which RANTES decreased at least about 5%, about 10%, or about 15% as compared to a reference level of RANTES, indicates cognitive impairment, such as for example, an indication of AD. Accordingly, methods are provided in which PDGF-BB decreased at least about 80%, about 85% or about 90% as compared to a reference level of PDGF-BB, indicates cognitive impairment, such as for example, an indication of severe AD.

TABLE 14

| | | | Protein |
|---|---|---|---|
| Protein | Alternate names | Class | ID |
| alpha-1 acid glycoprotein | | acute phase | |
| alpha-1 antitrypsin | | acute phase | |
| Ceruloplasmin | | acute phase | |
| Haptoglobin | | acute phase | |
| Hemopexin | | acute phase | |
| Hemoxygenase | | acute phase | |
| plasminogen activator inhibitor-1 | PAI-1 | acute phase | |
| serum amyloid A | SAA | acute phase | |
| serum amyloid P | SAP | acute phase | |
| 4-11313 ligand | 4-1BBL/CD137L | apoptosis | P41273 |
| BAFF | TALL-1 | apoptosis | Q9Y275 |
| soluble TRAIL receptor 3 | TRAIL sR3/TNFR S10C | apoptosis | 014755 |
| soluble TRAIL receptor 4 | TRAIL sR4/TNFR S10D | apoptosis | Q9UBN6 |
| TNF-related death ligand 1a | TRDL-1a/APRIL | apoptosis | AF046888 |
| TNFSF-14 | LIGHT | apoptosis | 043557 |
| TRAIL | Apo2L | apoptosis | P50591 |
| BCA-1 | BLC | chemokine | 043927 |
| CCL-28 | CCK-1 | chemokine | |
| cutaneous T cell attracting chemokine | CTACK, CCL27 | chemokine | Qgz1X0 |
| ENA-78 | | chemokine | P42830 |
| Eotaxin-1 | | chemokine | P51671 |
| Eotaxin-2 | MPIF-2 | chemokine | 000175 |
| Eotaxin-3 | CCL26 | chemokine | Q9Y258 |
| Fractalkine | neurotactin | chemokine | P78423 |
| Granulocyte chemotactic protein 2 | GCP-2 | chemokine | P80162 |
| GRO alpha | MGSA | chemokine | P09341 |
| GRO beta | MIP-2alpha | chemokine | P19875 |
| GRO gamma | MIP-2beta | chemokine | P19876 |
| haemoinfiltrate CC chemokine 1 | HCC-1 | chemokine | Q16627 |
| haemoinfiltrate CC chemokine 4 | HCC-4/CCL16 | chemokine | 015476 |
| I-309 | TCA-3/CCL-1 | chemokine | P22362 |
| IFNgamma inducible protein-10 | IP-10 | chemokine | P02778 |
| IFN-inducible T cell alpha chemokine | I-TAC/CXCL11 | chemokine | AF030514 |
| interleukin-8 | IL-8/NAP-1 | chemokine | P10145 |
| leucocyte cell-derived chemotaxin-2 | LECT2 | chemokine | |
| Lungkine | CXCL-15/WECHE | chemokine | |
| Lymphotactin | Lptn/ATAC | chemokine | P47992 |
| | | | MIP- |
| | | | 1alpha/ |
| | | | pLD78/ |
| macrophage inflammatory protein lalpha | CCL3 | chemokine | P10147 |
| macrophage inflammatory protein lbeta | MIP-lbeta/ACT-2/CCL4 | chemokine | P13236 |
| macrophage inflammatory protein ld | MIP-1d/CCL15/LKN-1 | chemokine | |
| macrophage inflammatory protein 1gamma | MIP-1gamma/CCL9/MIP- | chemokine | |
| | 3alpha/CCL20/ | | |
| macrophage inflammatory protein 3alpha | LARC | chemokine | P78556 |
| macrophage inflammatory protein 3beta | MIP-3beta/ELC/CCL19 | chemokine | Q99731 |
| macrophage-derived chemokine | MDC/STCP-1 | chemokine | 000626 |
| monocyte chemoattractant protein-1 | MCP-1/CCL2 | chemokine | P13500 |
| monocyte chemoattractant protein-2 | MCP-2/CCL8 | chemokine | P78388 |
| monocyte chemoattractant protein-3 | MCP-3/CCL7 | chemokine | P80098 |
| monocyte chemoattractant protein-4 | MCP-4/CCL13 | chemokine | Q99616 |
| monocyte chemoattractant protein-5 | MCP-5/CCL12 | chemokine | |
| monokine induced by IFN gamma | MIG | chemokine | Q07325 |
| mucosa-associated chemokine | MEC | chemokine | AF266504 |
| Myeloid progenitor inhibitory factor | MPIF/CKbeta8/CCL23 | chemokine | |
| platelet basic protein | PBP/CTAP-III/NAP-2 | chemokine | P02775 |
| platelet factor 4 | PF-4/CXCL4 | chemokine | P02776 |
| pulmonary activation regulated chemokine | PARC/CCL18/MIP-4 | chemokine | |
| RANTES | CCL5 | chemokine | P13501 |
| secondary lymphoid tissue chemokine | SLC/6Ckine | chemokine | 000585 |
| stromal cell derived factor 1 | SDF-1/CXCL12 | chemokine | P48061 |
| thymus activation regulated chemokine | TARC/CCL17 | chemokine | Q92583 |
| thymus expressed chemokine | TECK/CCL25 | chemokine | 015444 |
| Clq | | collectin | |
| mannose binding lectin | MBL | collectin | |
| surfactant protein A | SP-A | collectin | |
| surfactant protein D | SP-D | collectin | |
| C1 inhibitor | | complement | |
| C3a | | complement | |
| Cob binding protein | C4BP | complement | |
| C5a | | complement | |
| complement C3 | C3 | complement | |
| complement C5 | C5 | complement | |
| complement C8 | C8 | complement | |
| complement C9 | C9 | complement | |
| decay accelerating factor | DAF | complement | |
| Factor H | | complement | |
| membrane inhibitor of reactive lysis | MIRL/CD59 | complement | |
| Properdin | | complement | |
| soluble complement receptor 1 | sCR1 | complement | |
| soluble complement receptor 2 | sCR2 | complement | |
| cardiotrophin-1 | CT-1 | cytokine | Q16619 |
| CD27 | | cytokine | P26842 |
| CD27L | CD70 | cytokine | P32970 |
| CD30 | Ki-1 | cytokine | P28908 |
| CD30L | TNFSF8 | cytokine | P32971 |
| CD40L | TRAP/CD154 | cytokine | P29965 |
| interferon alpha | IFNalpha | cytokine | P01562 |
| interferon beta | IFNbeta | cytokine | P01574 |
| interferon gamma | IFNgamma | cytokine | P01579 |
| interferon omega | IFNomega | cytokine | P05000 |
| interferon-sensitive gene 15 | ISG-15 | cytokine | P05161 |
| Leptin | OB | cytokine | P41159 |
| leukemia inhibitory factor | LIF/CNDF | cytokine | P15018 |
| Lymphotoxin | LT/TNF beta | cytokine | P01374 |
| macrophage colony stimulating factor | M-CSF/CSF-1 | cytokine | P09603 |
| macrophage stimulating protein-alpha | MSPalpha/HGF1 | cytokine | P26927 |
| macrophage stimulating protein-beta | MSPbeta/HGF1 | cytokine | P26927 |
| migration inhibition factor | MIF/GIF | cytokine | P14174 |
| oncostatin M | OSM | cytokine | P13725 |
| RANKL | TRANCE/TNFSF-11 | cytokine | 014788 |
| soluble IL6 R complex | sIL6RC (gp130 + sIL6R) | cytokine | |
| soluble Fas ligand | sCD95L | cytokine | P48023 |
| TNF type I receptor | TNF-RI p55 | cytokine | P19438 |
| TNF type II receptor | TNF-R p75 | cytokine | P20333 |
| TNFSF-18 | GITRL/AITRL | cytokine | 095852 |
| tumor necrosis factor alpha | TNF-alpha/Apo3L/DR3-L/ | cytokine | P01375 |
| | TNFSF-12 | | |
| TWEAK | | cytokine | 043508 |
| acidic fibroblast growth factor | aFGF | growth factor | P05230 |
| activin beta A | | growth factor | P08476 |
| agouti related protein | AGRP | growth factor | AAB52240 |
| Amphiregulin | AR/SDGF | growth factor | P15514 |
| angiopoietin-like factor | ALF | growth factor | |
| basic fibroblast growth factor | bFGF | growth factor | P09038 |
| Betacellulin | | growth factor | P35070 |
| bone morphogenic protein 2 | BMP2 | growth factor | P12643 |
| bone morphogenic protein 4 | BMP4 | growth factor | |
| bone morphogenic protein 5 | BMP5 | growth factor | |
| bone morphogenic protein 6 | BMP6 | growth factor | |
| bone morphogenic protein 7 | BMP7 | growth factor | |
| cripto-1 | CRGF | growth factor | |
| epidermal growth factor | EGF | growth factor | P01133 |
| Erythropoietin | Epo | growth factor | |
| fibroblast growth factor 17 | FGF-17 | growth factor | |
| fibroblast growth factor 18 | FGF-18 | growth factor | |
| fibroblast growth factor 19 | FGF-19 | growth factor | |
| fibroblast growth factor 2 | FGF-2 | growth factor | |
| fibroblast growth factor 4 | FGF-4 | growth factor | |
| fibroblast growth factor 6 | FGF-6 | growth factor | |
| fibroblast growth factor 7 | FGF-7/KGF | growth factor | |
| fibroblast growth factor 8 | FGF-8 | growth factor | |
| fibroblast growth factor 9 | FGF-9 | growth factor | |
| Flt3 ligand | Flt L | growth factor | P49771 |
| Follistatin | FSP | growth factor | |
| Granulocyte colony stimulating factor | G-CSF | growth factor | P09919 |
| granulocyte/macrophage CSF | GM-CSF | growth factor | P04141 |
| growth and differentiation factor 11 | GDF-11 | growth factor | |
| growth and differentiation factor 15 | GDF-15 | growth factor | |
| growth arrest specific gene 6 | Gas-6 | growth factor | |
| heparin-binding epidermal growth factor | HB-EGF | growth factor | Q99075 |
| hepatocyte growth factor | HGF/SF | growth factor | P14210 |
| hepatopoietin A | HPTA/HRG alpha/ | growth factor | |
| | neuregulin | | |
| heregulin alpha | NDF/HRG beta/neuregulin/ | growth factor | |
| heregulin beta | NDF | growth factor | |
| IGF binding protein-1 | IGFBP-1 | growth factor | |
| IGF binding protein-2 | IGFBP-2 | growth factor | |
| IGF binding protein-3 | IGFBP-3 | growth factor | |
| IGF binding protein-4 | IGFBP-4 | growth factor | |
| inhibin A | | growth factor | |
| inhibin B | | growth factor | |
| insulin-like growth factor IA | IGF-IA | growth factor | P01343 |
| insulin-like growth factor IB | IGF-IB | growth factor | P05019 |
| insulin-like growth factor II | IGF-II | growth factor | P01344 |
| macrophage galatose-specific lectin 1 | MAC-1 | growth factor | |
| Neuritin | | growth factor | |
| Neurturin | | growth factor | |
| orexin A | | growth factor | |
| Osteonectin | SPARC | growth factor | |
| Osteoprotegrin | TNFRSF11B | growth factor | |
| placenta growth factor | PGIF | growth factor | |
| platelet derived growth factor alpha | PDGF-A | growth factor | P04085 |
| platelet derived growth factor beta | PDGF-B | growth factor | P01127 |
| pregnancy zone protein | | growth factor | |
| Prolactin | PRL | growth factor | P01236 |
| sensory and motor neuron-derived factor | SMDF | growth factor | |
| soluble GM-CSF receptor | sGM-CSF R | growth factor | P15509 |
| stem cell factor | SLF/SCF/kit ligand/MGF | growth factor | P21583 |
| Thrombopoietin | TPO/c-MPL ligand | growth factor | P40225 |
| thymic stromal lymphoprotein | TSLP | growth factor | |
| Thymopoietin | Tpo | growth factor | |
| transforming growth factor alpha | TGF-alpha | growth factor | P01135 |
| transforming growth factor beta 1 | TGF-beta1 | growth factor | P01137 |
| transforming growth factor beta 2 | TGF-beta2 | growth factor | P08112 |
| transforming growth factor beta 3 | TGF-beta3 | growth factor | P10600 |
| vascular endothelial growth factor | VEGF | growth factor | P15692 |
| interleukin-1 receptor antagonist | ILiRa | interleukin | P18510 |
| interleukin-10 | IL-10 | interleukin | P22301 |
| interleukin-11 | IL-11 | interleukin | P20809 |
| interleukin-12p35 | IL-12p35 | interleukin | P29459 |
| interleukin-12p40 | IL-12p40 | interleukin | P29460 |
| interleukin-13 | IL-13 | interleukin | P35225 |
| interleukin-14 | IL-14 | interleukin | L15344 |
| interleukin-15 | IL-15 | interleukin | P40933 |
| interleukin-16 | IL-16 | interleukin | Q14005 |
| interleukin-17 | IL-17 | interleukin | Q16552 |
| interleukin-18 | IL-18 | interleukin | Q14116 |
| interleukin-lalpha | IL-lal.pha | interleukin | P01583 |
| interleukin-lbeta | IL-lbeta | interleukin | P01584 |
| interleukin-2 | IL-2 | interleukin | P01585 |
| interleukin-3 | IL-3 | interleukin | P08700 |
| interleukin-4 | IL-4 | interleukin | P05112 |
| interleukin-5 | IL-5 | interleukin | P05113 |
| interleukin-6 | IL-6 | interleukin | P05231 |
| interleukin-7 | IL-7 | interleukin | P13232 |
| interleukin-9 | IL-9 | interleukin | P15248 |
| soluble interleukin-1 receptor I | sILIR/CD121a | interleukin | P14778 |
| soluble interleukin-1 receptor II | sIL1R/CD121b | interleukin | P27930 |
| soluble interleukin-2 receptor | IL-2R/CD25 | interleukin | P01589 |
| soluble interleukin-5 receptor | sIL-5R/CD125 | interleukin | Q01344 |
| soluble interleukin-6 receptor | sIL-6R/CD126 | interleukin | P08887 |
| soluble interleukin-7 receptor | sIL-7R/CD127 | interleukin | P16871 |
| soluble interleukin-9 receptor | sIL-9R | interleukin | PQ01113 |
| AD7C | NTP | neuronal | AF010144 |
| alpha synuclein | | neuronal | AAH13293 |
| GAP-43 | | neuronal | |
| Neurofilament | | neuronal | |
| Synaptogamin | | neuronal | |
| Synaptophysin | | neuronal | |
| tau P199 | | neuronal | |
| brain derived neurotrophic factor | BDNF | neurotrophin | P23560 |
| ciliary neurotrophic factor | CNTF | neurotrophin | P26441 |
| glial derived neurotrophic factor | GDNF | neurotrophin | P39905 |
| nerve growth factor | NGF | neurotrophin | P01138 |
| neurotrophin 3 | NT-3 | neurotrophin | P20783 |
| neurotrophin 4 | NT-4 | neurotrophin | P34130 |
| soluble CNTF receptor | sCNTFR | neurotrophin | P26992 |
| alpha2-macroglobulin | alpha 2M | others | |
| Alzheimer associated protein | ALZAS | others | |
| amyloid beta protein | Abeta 1-x | others | |
| apolipoprotein A | apoA | others | |
| apolipoprotein B | apoB | others | |
| apolipoprotein D | apoD | others | |
| apolipoprotein E | apoE | others | |
| apolipoprotein J | apoD/clusterin | others | |
| C reactive protein | CRP | others | |
| clara cell protein | CC16 | others | |
| glial fibrillary acidic protein | GFAP | others | |
| Melanotransferrin | | others | |
| soluble transferring receptor | TfR | others | |
| Thrombomodulin | | others | |
| Thrombospondin | Tsp | others | |
| tissue transglutaminase | | others | |
| Transferrin | | others | |
| alpha 1-antichymotrypsin | ACT | protease | NP001076 |
| Clr | | protease | |
| Cls | | protease | |
| complement C2 | C2 | protease | |
| Factor B | | protease | |
| Factor D | adipsin | protease | |
| FactorI | | protease | |
| Kallikrein | | protease | |
| MBL-associated serine protease 1 | MASP-1 | protease | |
| MBL-associated serine protease 2 | MASP-2 | protease | |
| Neuroserpin | | protease | AAH18043 |
| secretory leukocyte protease inhibitor | SLPI | protease | |
| Angiogenin | | vascular | |
| Angiostatin | | vascular | P00747 |
| Endostatin | | vascular | |
| Endothelin | | vascular | |
| soluble E selectin | s E selectin | vascular | |
| vascular endothelial growth inhibitor | VEGI | vascular | |

### Example 13

This example describes methods useful for measuring the levels of AD biomarkers and/or analyzing data regarding measurements of the levels of AD biomarkers and/or correlating data based on the measurements of the levels of AD biomarkers and/or identifying AD biomarkers by analyzing and/or correlating data based on the measurements of the levels of AD biomarkers obtained from biological samples from subjects across different test centers. These methods are also applicable to biological samples obtained from an individual and/or single collection center. The methods are designed to minimize or reduce test center variability resulting from collection procedures and/or storage and handling conditions. This example, along with Example 14, provides methods for identifying additional biomarkers that are useful in the detection of AD, including markers which provide a high degree of sensitivity (calculated as the number of AD samples in the AD cluster divided by the total number of AD samples used in the experiment) and specificity (calculated as the number of controls in the control cluster divided by total number of controls used in the experiment for diagnosing AD), as well as identifying such biomarkers.

Collection procedures as well as storage and handling conditions can introduce variability in the concentration of biomarkers measured in biological samples, such as plasma, of AD and Control Subjects. This in turn could cause misclassification of subjects without appropriate normalization and/or standardization and/or controls. For example, protein concentrations may be affected, in part, by whether a particular plasma sample is platelet rich or platelet poor. In general, plasma samples that are platelet rich will have greater quantitative levels of many biomarkers, while samples that are platelet poor will have reduced quantitative levels of many biomarkers (as compared to appropriate controls, for example population controls). For example, the concentration of BDNF, which is tightly held within platelets, was measured as a surrogate for platelet degranulation and therefore the release of BDNF from platelets. It was observed that carefully prepared platelet poor plasma has a concentration of BDNF that is equivalent to 10 pg/ml whereas platelet rich preparations of plasma can have concentrations as high as 20 ng/ml. The correlation of BDNF measured by ELISA and BDNF measured by spotted filter antibody array has an r=0.679, with p<0.0001. The samples used in the experimental design were prepared in a manner such that they did not include platelet poor preparation of BDNF, as these are not representative of plasma collection in common practice.

In some examples, plasma is used as the biological sample for the methods disclosed herein rather than serum. Plasma was used in the methods of Example 3, and Examples 12-15. This is due, in part, to the variables involved in the blood clotting process used to make serum. These variables may lead to varying degrees of proteolysis of biomarkers contained in the serum. Also, if plasma is used, there is less chance of inadvertently removing a protein of interest. If large amounts of fibrinogen or albumin do present a problem, there are depletion kits publicly available to deplete the plasma of these proteins, although if this is done, associated proteins may be removed as well. If depletion kits are used, appropriate controls to monitor removal of the associated proteins may be used in the methods.

Sterile blood collection tubes that are pre-loaded with protease inhibitors, as well as a self-contained system for removing red blood cells and platelets are publicly available. See for example, the Beckton Dickenson Company product lists at:

bd.com/vacutainer/products/venous/ordering_info_tubes.asp.

The protocol below is one illustrative example of sample collection procedures.

Becton Dickenson BD P100 tubes are stored at 4° C., until use. A full 8.5 mL of blood is collected to produce about 2.5-3 mL of plasma. Immediately after collection, the tube is inverted 8-10 times to mix the protease inhibitors and anticoagulent with the blood sample. The tube is placed in wet ice before centrifuging. (Centrifugation should be done within 30 minutes of collection). The tubes are centrifuged at 2000-3000 RCF at 4° C. for 15 min. (See BD P100 package insert for converting rpm to RCF). Do not exceed 3000 g, or 10,000 RCF.

Within 30 minutes of centrifugation, the plasma is transferred in 1-mL aliquots to pre-labeled Fisherbrand 4-mL self-standing cryovials (Fisher Scientific # 0566966) and immediately placed on dry ice. Aliquots are frozen at ±80° C. until used. (Avoid freeze-thaw cycles). To remove microplatelets, the plasma is transferred to a different centrifuge tube, and is centrifuged at 12,000 g at 4° C. for 15 min.

The objective of this experiment, in part, was to determine methods, including identification of appropriate controls, for use in analyzing data that minimize individual variations in the immune response and variations produced by collection and storage conditions while identifying AD subjects with a high degree of specificity and sensitivity.

The methods used in the experiments were the same as described herein in Example 3 with filter based antibody arrays consisting of 120 antibodies specific for the proteins, that is biomarkers, listed in Table 15. In some previous experiments using filter based antibody arrays of 120 antibodies specific for the biomarkers listed in Table 15 (the designation of “_{—}1” after each biomarker name in Tables 15, 16A1-16A2 and 16B, 17A1-17A2 and 17B, 18A1-18A2 and 18B, and 19A-19B is a function of the program and is not part of the name of each biomarker) when a signal was not detectable, it was not clear if this was a false negative result (for example, due to problems with the use of certain of the reagents) or a true negative result. In the following experiments, due to improvements made by the manufacturer of the reagents (RayBiotech), it was determined that a signal could be detected for all of the 120 proteins screened using the antibody arrays. This improvement in reagents resulted in identification of additional biomarkers (as shown in Example 14) for use in the methods as disclosed herein, such as for example, in methods for aiding in the diagnosis of and/or diagnosing AD, which biomarkers may or may not have been detectable in previous experiments.

In this experiment, the levels of the 120 biomarkers listed in Table 15 were measured for biological samples collected at five different Alzheimer's centers (n=34, mean age=74, Mean MMSE=20) including 16 samples collected 1.5 yrs apart from 8 subjects with AD, who were later confirmed by autopsy to have AD, were compared to controls, for example, age matched controls collected from two centers (n=17) and other non-AD neurodegenerative age-matched controls (n=16) consisting of 4 subjects diagnosed with Parkinson's disease, and 12 subjects diagnosed with peripheral neuropathy. Power calculations show that 10 samples of autopsy confirmed AD samples are necessary to have an Alpha of 0.001 and power of 0.999.

Experimental data for all 120 biomarkers were extracted using Imagene software licensed from Biodiscovery. The extracted data was then normalized to the positive control for the experiment spotted on the blot. An example of a positive control is IgG. The data for each individual biomarker was then normalized to the median concentration of all 120 proteins measured by the antibody array. The Significance analysis of microarrays (SAM) was used to determine significance of each biomarker. This method for normalizing data extracted from a blot experiment minimizes or reduces variability due to the fact that individual samples can have slightly higher or lower levels of proteins based on the individual's immune response status. Following the determination of significance using SAM, the biomarkers with p-values less than or equal to 0.1% (53 markers) were used for cluster analysis to classify AD from controls. (See Tables 20A (biomarkers that are positively correlated) and 20B (biomarkers that are negatively correlated for the markers listed that have a p-value % of about 0.1). All biomarkers with p-values less than or equal to 5% (Tables 16A1-16A2 and 16B) were all used in cluster analysis to classify samples as AD based on the controls used. Results of analysis of extracted data that were normalized as described above are disclosed in Example 14 and Tables 20A-20B (unclustered, and in order of highest ranked biomarker to lowest ranked biomarker, significantly increased (20A) or decreased (20B) in AD compared to age-matched normal controls plus other non-AD forms of neurodegeneration, such as PD an PN (that is, as compared to all controls). The columns from left to right for Tables 20A-20B are biomarker Name, Score (d), fold change and p-value (%). Tables 16A1-16A2 and 16B as described in Example 14 show an additional analysis of data for biomarkers having a p-value of greater than 0.1% and less than 5%.

### Example 14

This example describes methods for identifying AD biomarkers that are either increased or decreased in individuals diagnosed with AD compared to healthy age matched controls and/or neurodegenerative age matched controls that are non-AD, that is, non-AD neurodegenerative controls, such as Parkinson's Disease (PD), and peripheral neuropathy (PN). This is important because AD is a neurodegenerative disease, and it is advantageous to identify biomarker patterns of neurodegeneration associated with AD, in terms of identification of biomarkers that are either decreased or increased with respect to an appropriate control(s), that are unique to AD and/or distinguishable from other non-AD forms of neurodegeneration, such as for example PD and PN, in the same age group, as well as with respect to healthy age-matched controls.

Previous experiments (see Example 3) determined that any one or more of the following biomarkers could be used for the detection of AD: GCSF; IFN-g; IGFBP-1; BMP-6; BMP-4; Eotaxin-2; IGFBP-2; TARC; RANTES; ANG; PARC; Acrp30; AgRP(ART); TIMP-1; TIMP-2; ICAM-1; TRAIL R3; uPAR; IGFBP-4; LEPTIN(OB); PDGF-BB; EGF; BDNF; NT-3; NAP-2; IL-1ra; MSP-a; SCF; TGF-b3; TNF-b; MIP-1d; IL-3; FGF-6; IL-6 R; sTNF R11; AXL; bFGF; FGF-4; CNTF; MCP-1; MIP-1b; TPO; VEGF-B; IL-8; FAS; EGF-R. Based upon the experimental conditions and analysis described in Example 13, additional biomarkers useful for detecting AD were identified. The measured values for the biomarkers from Table 15 were subjected to hierarchical clustering based on classification of samples with normalized concentration surveyed. Based upon the clustering analysis, the proteins were segregated into 9 classes of similarities based on correlation. Biomarkers with greater than a 5% p value (%) were eliminated from the analysis. Sensitivity of the classification is calculated as the number of AD samples in the AD cluster divided by the total number of AD samples used in the experiment (in this case 31/34=91%). Specificity is calculated as the number of controls in the control cluster divided by total number of controls used in the experiment (in this case 31/33=94%).

Tables 20A-20B provide a listing of biomarkers as described in Example 13. Tables 16A1-16A2 and 16B provide a listing of biomarkers (clustered by methods as described above) in order of highest ranked biomarker to lowest ranked biomarker within each cluster based on score value) that are significantly increased (16A1-16A2) or decreased (16B) in AD compared to age-matched normal controls plus other non-AD forms of neurodegeneration, such as for example PD and PN (that is, as compared to all controls). The columns from left to right for Table 16A1-16A2 and 16B are: biomarker name; Score(d); Fold change; q-value(%) and cluster number. Significance analysis of microarrays is discussed in for example Tusher et al., 2001, PNAS, vol. 98:5116. Any one or more of the biomarkers listed in Table 16A1-16A2 and 16B can be used in the methods disclosed herein, such as for examples, methods for aiding in the diagnosis of or diagnosing AD. As described herein, multiple AD diagnosis biomarkers may be selected from the AD diagnosis biomarkers disclosed in Tables 16A1-16A2 and 16B by selecting for cluster diversity. The highest ranked biomarkers from each of the 9 clusters shown in Tables 16A1-16A2 and 16B (both positively correlated and negatively correlated) are: BTC (cluster 0); SDF-1 (cluster 1); MCP-2 (cluster 2); IFN-gamma (cluster 3); IGFBP-4 (cluster 4); IGF-1SR (cluster 5); IL-8 (cluster 6); GM-CSF (cluster 7); and ANG-2 (cluster 8). In some examples, biomarkers for use in the methods disclosed herein, such as for example, methods for aiding in the diagnosis of AD or diagnosing AD, include at least one marker selected from the group consisting of BTC; SDF-1; MCP-2; IFN-gamma; IGFBP-4; IGF-1SR; IL-8; GM-CSF; and ANG-2 or at least one marker from Tables 20A-20B. In some examples, additional biomarkers for use in the methods disclosed herein, such as for example, methods for aiding in the diagnosis of AD or diagnosing AD, include biomarkers that correlate with one or more of BTC; SDF-1; MCP-2; IFN-gamma; IGFBP-4; IGF-1SR; IL-8; GM-CSF; and ANG-2, that is, such biomarkers that have a Correlation: greater than 90% (r=0.9 to r=0.99); and a P-value less than 0.001 up to 0.05.

In some examples, biomarkers for use in the methods disclosed herein, such as for example, methods for aiding in the diagnosis of AD or diagnosing AD include two or more markers selected from the group consisting of BTC; SDF-1; MCP-2; IFN-gamma; IGFBP-4; IGF-1SR; IL-8; GM-CSF; and ANG-2. In some examples, biomarkers for use in the methods disclosed herein, such as for example, methods for aiding in the diagnosis of AD or diagnosing AD include markers comprising BTC; SDF-1; MCP-2; IFN-gamma; IGFBP-4; IGF-1SR; IL-8; GM-CSF; and ANG-2. In other examples, the top ranked 2, 3, 4, or 5 biomarkers from one or more clusters represented in Tables 16A1-16A2 and 16B are selected for use in the methods as disclosed herein.

Tables 17A1-17A2 and 17B provide a listing of biomarkers (not clustered and in order of highest ranked biomarker to lowest ranked biomarker based on score value) that are significantly increased (17A1-17A2) or decreased (17B) in AD compared to healthy age-matched controls. The columns from left to right in Tables 17A1-17A2 and 17B, Tables 18A1-18A2 and 18B, and Tables 19A-19B are Biomarker name, Score(d); Fold change; and q-value(%). Based on Tables 17A1-17A2 and 17B, identified biomarkers that are significantly increased in AD as compared to healthy age-matched controls include, but are not limited to (in descending order based on score): BTC; ANG-2; MIF; IGFBP-6; spg130; CTACK; IGFBP3; MIP-1a; TRAIL R4; IL-12 p40; AR; NT-4; VEGF-D; OSM; OST; IL-11; sTNF R1; I-TAC; Eotaxin; TECK; PIGF; bNGF; Lymphotactin; MIP-3b; HCC-4; ICAM-3; DTK; IL-1 RI; IGF-1 SR; GRO; GITR-Light; HGF; IL-1R4/ST; IL-2 Ra; ENA-78; and FGF-9. Based on Tables 17A1-17A2 and 17B, identified biomarkers that are significantly decreased in AD as compared to healthy age-matched controls include, but are not limited to (in descending order based on score): MCP-2; M-CSF; MCP-3; MDC; MCP-4; IL-1b; IL-4; IL-1a; BLC; CK b8-1; IL-2; IL-15; MIP3a; MIG; SCF; IL-6; IL-16; Eotaxin-3; 1-309; TGF-beta; TGF-alpha; GDNF; LIGHT; SDF; IFG-1; Fractalkine; IL-5; Fit-3 ligand; GM-CSF; and GCP-2. Any one or more of the biomarkers listed in Tables 17A1-17A2 and 17B can be used in the methods disclosed herein, such as for example, for aiding in the diagnosis of or diagnosing AD.

Tables 18A1-18A2 and 18B provide a listing of biomarkers (not clustered and in order of highest ranked biomarker to lowest ranked biomarker based on score value) that are significantly increased (18A1-18A2) or decreased (18B) in AD compared to age-matched degenerative controls. Based on Tables 18A1-18A2 and 18B, identified biomarkers that are significantly increased in AD as compared to age-matched other non-AD neurodegenerative controls include, but are not limited to (in descending order based on score): TRAIL R4; Eotaxin; IL-12 p40; BTC-1; MIF; OST; MIP-1a; sTNF R1; IL-11; Lymphotactin; NT-4; VEFG-D; HGF; IGFBP3; IGFBP-1; OSM; IL-1R1; PIGF; IGF-1 SR; CCL-28; IL-2 Ra; IL-12 p′70; GRO; IGFBP-6; IL-17; CTACK; I-TAC; ICAM-3; ANG-2; MIP-3b; FGF-9; HCC-4; IL-1R4/ST; GITR; and DTK. Based on Tables 18A1-18A2 and 18B, identified biomarkers that are significantly decreased in AD as compared to age-matched other non-AD neurodegenerative controls include, but are not limited to (in descending order based on score): MCP-2; M-CSF; MCP-3; MDC; MCP-4; IL-1b; IL-4; IL-1a; BLC; CKb8-1; IL-2; IL-15; MIP3a; MIG; SCF; IL-6; IL-16; Eotaxin-3; 1-309; TGF-beta; TNF-alpha; GDNF; LIGHT; SDF-1; IFG-1; Fractalkine; IL-5; Fit-3 Ligand; GM-CSF; and GCP-2. Any one or more of the biomarkers listed in Tables 18A1-18A2 and 18B can be used in the methods disclosed herein, such as for example, methods for aiding in the diagnosis of or diagnosing AD.

Tables 19A-19B provide a listing of biomarkers (not clustered and in order of highest ranked biomarker to lowest ranked biomarker based on score value) that are significantly increased (19A) or decreased (19B) in AD plus other non-AD neurodegenerative controls with reference to age matched controls. Any one or more of the biomarkers listed in Tables 19A-19B can be used in the methods disclosed herein, such as for example, methods for aiding in the diagnosis of or diagnosing neurodegenerative diseases, including AD. In other examples, the top ranked 2, 3, 4, or 5 biomarkers listed in Tables 19A-19B are selected for use in the methods as disclosed herein. In some examples, additional biomarkers for use in the methods disclosed herein, such as for example, methods for aiding in the diagnosis of AD or diagnosing AD, include biomarkers that correlate with the top ranked 1, 2, 3, 4, or 5 biomarkers listed in Tables 19A-19B, that is, such biomarkers that have a Correlation: greater than 90% (r=0.9 to r=0.99); and a P-value less than 0.001 up to 0.05.

As will be understood by the skilled artisan, biomarkers disclosed herein in the Examples and Tables can be selected for use in the methods disclosed herein depending on the type of measurement desired. For example, any one or more of the markers selected from the group consisting of the markers listed in Table 14 and/or Table 15 can be used to aid in the diagnosis of AD or for diagnosing AD. In some examples, biomarkers from Table 14 and/or Table 15 are selected for use in the methods disclosed herein based on the following criteria: Correlation: greater than 90% (r=0.9 to r=0.99); P-value less than 0.001 up to 0.05; Fold change greater than 20%; and a Score greater than 1 (for markers that increase, that is, that are positively correlated) or less than 1 (for markers that decrease, that is, that are negatively correlated).

In other examples, one or more markers selected from the group consisting of GCSF; IFN-g; IGFBP-1; BMP-6; BMP-4; Eotaxin-2; IGFBP-2; TARC; RANTES; ANG; PARC; Acrp30; AgRP(ART); TIMP-1; TIMP-2; ICAM-1; TRAIL R3; uPAR; IGFBP-4; LEPTIN(OB); PDGF-BB; EGF; BDNF; NT-3; NAP-2; IL-1ra; MSP-a; SCF; TGF-b3; TNF-b; MIP-1d; IL-3; FGF-6; IL-6 R; sTNF R11; AXL; bFGF; FGF-4; CNTF; MCP-1; MIP-1b; TPO; VEGF-B; IL-8; FAS; and EGF-R can be used in the methods disclosed herein, such as, for example, to aid in the diagnosis of AD or for the diagnosis of AD. In other examples, one or more biomarkers selected from Tables 19A-19B can be used to aid in the detection of general neurodegenerative disorders (including AD) and/or to diagnose neurodegenerative disorders generally while one or more biomarkers selected from Tables 16A1-16A2 and 16B can be used to aid in the diagnosis of AD or to diagnose AD and/or distinguish AD from other non-AD neurodegenerative diseases. In other examples, one or more biomarkers from Tables 17A1-17A2 and 17B or Tables 18A1-18A2 and 18B can be used to aid in the diagnosis of AD or to diagnose AD.

In addition to the biomarkers identified above, additional biomarkers can be identified by the methods described herein and methods known in the art. The parameters for selection of additional biomarkers are as follows:

Correlation: greater than 90% (r=0.9 to r=0.99);

P-value less than 0.001 up to 0.05;

Fold change greater than 20%; and

a Score greater than 1 (for markers that increase) or less than 1 (for markers that decrease).

### Example 15

This example provides the biomarkers for aiding in the diagnosis of or diagnosing AD identified in two different experiments (single collection center and multi-collection center) as being significant.

Additional biomarkers, sTNF RII; MSP-alpha; uPAR; TPO; MIP-1beta; VEGF-beta; FAS; MCP-1; NAP-2; ICAM-1; TRAIL R3; PARC; ANG; IL-3; MIP-1delta; IFN-gamma; IL-8; and FGF-6 were identified as significant in both the experiment from a single collection center (see Example 3) and the multi-test center experiment (Examples 12-13) that was normalized as described in Examples 12-13. Of these 18 biomarkers, two, IFN-gamma and IL-8, also appear in Tables 16A1-16A2 and 16B as the highest ranked biomarker from cluster 3 and cluster 6, respectively. Accordingly, biomarkers for use in the methods of the present invention for aiding in the diagnosis of or diagnosing AD include IFN-gamma and/or IL-8. It was found that the following two biomarkers were useful as normalization controls in the methods of the present invention for aiding in the diagnosis of or diagnosing AD: TGF-beta and TGF-beta3. Accordingly, biomarkers for use in the methods of the present invention, such as for example, for aiding in the diagnosis of or diagnosing AD include TGF-beta and/or TGF-beta3 as normalization controls.

TABLE 15
List of Biomarkers

ANG_1
BDNF_1
BLC_1
BMP-4_1
BMP-6_1
CK b8-1_1
CNTF_1
EGF_1
Eotaxin_1
Eotaxin-2_1
Eotaxin-3_1
FGF-6_1
FGF-7_1
Fit-3 Ligand_1
Fractalkine_1
GCP-2_1
GDNF_1
GM-CSF_1
I-309_1
IFN-g_1
IGF-1_1
IGFBP-1_1
IGFBP-2_1
IGFBP-4_1
IL-10_1
IL-13_1
IL-15_1
IL-16_1
IL-1a_1
IL-1b_1
IL-1ra_1
IL-2_1
IL-3_1
IL-4_1
IL-5_1
IL-6_1
IL-7_1
LEPTIN(OB)_1
LIGHT_1
MCP-1_1
MCP-2_1
MCP-3_1
MCP-4_1
M-CSF_1
MDC_1
MIG_1
MIP-1d_1
MIP-3a_1
NAP-2_1
NT-3_1
PARC_1
PDGF-BB_1
RANTES_1
SCF_1
SDF-1_1
TARC_1
TGF-b_1
TGF-b3_1
TNF-a_1
TNF-b_1
Acrp30_1
AgRP(ART)_1
ANG-2_1
AR_1
AXL_1
bFGF
b-NGF_1
BTC_1
CCL-28_1
CTACK_1
DTK_1
EGF-R_1
ENA-78_1
FAS_1
FGF-4_1
FGF-9_1
GCSF_1
GITR_1
GITR-Light_1
GRO_1
GRO-a_1
HCC-4_1
HGF_1
ICAM-1_1
ICAM-3_1
IGF-1 SR
IGFBP3_1
IGFBP-6_1
IL-1 RI_1
IL-11_1
IL-12 p40_1
IL-12 p70_1
IL-17_1
IL-1R4/ST2_1
IL-2 Ra_1
IL-6 R_1
IL-8_1
I-TAC_1
Lymphotactin_1
MIF_1
MIP-1a_1
MIP-1b_1
MIP-3b_1
MSP-a_1
NT-4_1
OSM_1
OST_1
PIGF_1
spg130_1
sTNF RI_1
sTNF RII_1
TECK_1
TIMP-1_1
TIMP-2_1
TPO_1
TRAIL
R3_1
TRAIL
R4_1
uPAR_1
VEGF-B_1
VEGF-D_1

### Example 15

Example 16 discloses the identification of biomarkers found to significantly correlate with MMSE scores (from 8 to 28) of AD subjects as shown below in Table 21. Therefore, Lymphotactin and IL-11 are useful for detection of early to mild AD and for the staging and progression of the disease. Lymphotactin and/or IL-11 can be used alone or together with other AD biomarkers, including those described herein in the methods disclosed herein. Accordingly, provided herein are methods for stratifying AD as well as monitoring the progress of AD that comprise comparing a measured level of Lymphotactin and/or IL-11 in a biological fluid sample, such as plasma, from an individual to a reference level for the biomarker.

TABLE 21
Correlation Coefficient
Hypothesized Correlation = 0

| | Cor- | | | P- | 95% | 95% |
|---|---|---|---|---|---|---|
| | relation | Count | Z-Value | Value | Lower | Upper |
| MMSE, IL-11_1 | .529 | 35 | 3.329 | .0009 | .237 | .733 |
| MMSE, | .516 | 35 | 3.226 | .0013 | .220 | .724 |
| Lymphotactin_1 | | | | | | |
| IL-11_1, | .488 | 35 | 3.015 | .0026 | .184 | .706 |
| Lymphotactin_1 | | | | | | |

TABLE 16A1

| Name | Score(d) | Fold Change | q-value (%) | Cluste |
|---|---|---|---|---|
| BTC_1 | 5.280599 | 2.30404 | 0.102881 | 0 |
| TRAIL R4_{—} | 4.18957 | 4.38847 | 0.102881 | 0 |
| MIF_1 | 3.78626 | 2.46763 | 0.102881 | 0 |
| MIP-1a_1 | 3.671968 | 2.04509 | 0.102881 | 0 |
| sTNF RII_{—} | 3.57664 | 1.81136 | 0.102881 | 0 |
| MSP-a_1 | 3.532718 | 2.23649 | 0.102881 | 0 |
| OST_1 | 3.519536 | 2.85493 | 0.102881 | 0 |
| uPAR_1 | 3.42578 | 3.10753 | 0.102881 | 0 |
| TPO_1 | 3.260328 | 2.04533 | 0.102881 | 0 |
| NT-4_1 | 3.182778 | 2.48474 | 0.102881 | 0 |
| MIP-1b_1 | 3.119065 | 2.07252 | 0.102881 | 0 |
| NAP-2_1 | 2.970365 | 1.51262 | 0.102881 | 0 |
| ICAM-1_1 | 2.949073 | 1.6633 | 0.102881 | 0 |
| IGFBP3_1 | 2.868921 | 1.68668 | 0.102881 | 0 |
| TRAIL R3_{—} | 2.808197 | 1.85516 | 0.102881 | 0 |
| Eotaxin_1 | 2.747874 | 2.23776 | 0.102881 | 0 |
| VEGF-B_1 | 2.73066 | 1.94657 | 0.102881 | 0 |
| PARC_1 | 2.703205 | 1.59801 | 0.102881 | 0 |
| sTNF RI_1 | 2.628389 | 2.27051 | 0.102881 | 0 |
| PIGF_1 | 2.59266 | 2.46572 | 0.102881 | 0 |
| OSM_1 | 2.548107 | 1.79103 | 0.102881 | 0 |
| ANG_1 | 2.527071 | 1.38167 | 0.102881 | 0 |
| FAS_1 | 2.522175 | 1.42939 | 0.102881 | 0 |
| VEGF-D_1 | 2.453761 | 3.08586 | 0.102881 | 0 |
| Acrp30_1 | 2.277494 | 2.1151 | 0.102881 | 0 |
| TIMP-1_1 | 1.815742 | 1.3765 | 0.102881 | 0 |
| TIMP-2_1 | 1.768441 | 1.37666 | 0.102881 | 0 |
| MIP-3b_1 | 1.516186 | 1.55797 | 0.290698 | 0 |
| RANTES_{—} | 1.482515 | 1.29415 | 0.290698 | 0 |
| EGF-R_1 | 1.461975 | 1.24406 | 0.362319 | 0 |
| CCL-28_1 | 1.332609 | 2.09378 | 0.362319 | 0 |
| GCSF_1 | 1.248565 | 1.39107 | 0.531915 | 0 |
| bFGF | 1.135651 | 1.19806 | 0.687285 | 0 |
| b-NGF_1 | 1.018717 | 1.22647 | 0.948845 | 0 |
| TGF-b3_1 | 1.000846 | 1.16675 | 0.948845 | 3 |
| IGF-1 SR | 2.154497 | 2.01788 | 0.102881 | 5 |
| GRO_1 | 1.12464 | 1.34176 | 0.687285 | 5 |
| FGF-9_1 | 0.908764 | 1.34736 | 1.257862 | 5 |
| GITR-Light | 0.891591 | 1.23962 | 1.323988 | 5 |
| IL-8_1 | 4.611751 | 2.30142 | 0.102881 | 6 |
| IL-12 p40_{—} | 4.397923 | 2.30237 | 0.102881 | 6 |
| IL-11_1 | 3.428231 | 3.16541 | 0.102881 | 6 |
| Lymphotac | 2.655294 | 1.92588 | 0.102881 | 6 |
| IL-1 RI_1 | 2.299796 | 2.69797 | 0.102881 | 6 |
| CTACK_1 | 2.166969 | 1.4123 | 0.102881 | 6 |
| HGF_1 | 1.917834 | 2.11589 | 0.102881 | 6 |
| I-TAC_1 | 1.761741 | 1.75813 | 0.102881 | 6 |
| ICAM-3_1 | 1.647733 | 1.63994 | 0.102881 | 6 |
| IL-2 Ra_1 | 1.517361 | 1.75028 | 0.290698 | 6 |
| DTK_1 | 1.334052 | 1.36685 | 0.362319 | 6 |
| IL-12 p70_{—} | 1.136177 | 1.52347 | 0.687285 | 6 |

TABLE 16A2

| Name | Score(d) | Fold Change | q-value (%) | Cluster |
|---|---|---|---|---|
| IL-17_1 | 0.973182 | 1.5033 | 0.948845 | 6 |
| ANG-2_1 | 2.573094 | 1.48217 | 0.102881 | 8 |
| IGFBP-6_1 | 2.559164 | 1.49096 | 0.102881 | 8 |
| IL-6 R_1 | 2.308765 | 1.42281 | 0.102881 | 8 |
| IGFBP-1_1 | 1.641212 | 1.3909 | 0.102881 | 8 |
| AR_1 | 1.388841 | 1.31995 | 0.362319 | 8 |
| IGFBP-2_1 | 1.313148 | 1.18336 | 0.362319 | 8 |
| HCC-4_1 | 1.301826 | 1.48316 | 0.362319 | 8 |
| IL-1R4/ST | 0.973381 | 1.28961 | 0.948845 | 8 |

TABLE 16B

| Name | Score(d) | Fold Change | q-value (%) | Cluster |
|---|---|---|---|---|
| SDF-1_1 | −3.717529 | 0.51302 | 0.102881 | 1 |
| TNF-a_1 | −3.502517 | 0.52906 | 0.102881 | 1 |
| TARC_1 | −2.327413 | 0.47705 | 0.102881 | 1 |
| TNF-b_1 | −1.156171 | 0.86239 | 1.121795 | 1 |
| MCP-2_1 | −5.829911 | 0.25732 | 0.102881 | 2 |
| M-CSF_1 | −5.008296 | 0.42889 | 0.102881 | 2 |
| IL-1a_1 | −4.92065 | 0.29231 | 0.102881 | 2 |
| MDC_1 | −4.362592 | 0.48973 | 0.102881 | 2 |
| MCP-3_1 | −4.034665 | 0.36994 | 0.102881 | 2 |
| BLC_1 | −3.624823 | 0.54297 | 0.102881 | 2 |
| MCP-4_1 | −3.391387 | 0.33264 | 0.102881 | 2 |
| Eotaxin-3_{—} [symbol] | −3.378874 | 0.50745 | 0.102881 | 2 |
| IL-3_1 | −3.292671 | 0.45124 | 0.102881 | 2 |
| IL-1b_1 | −3.2351 | 0.33216 | 0.102881 | 2 |
| IL-16_1 | −3.112419 | 0.26418 | 0.102881 | 2 |
| IL-2_1 | −3.091275 | 0.39923 | 0.102881 | 2 |
| FGF-6_1 | −2.995265 | 0.60629 | 0.102881 | 2 |
| IL-15_1 | −2.990886 | 0.2798 | 0.102881 | 2 |
| IL-4_1 | −2.909983 | 0.56937 | 0.102881 | 2 |
| GDNF_1 | −2.898614 | 0.57687 | 0.102881 | 2 |
| I-309_1 | −2.813435 | 0.58059 | 0.102881 | 2 |
| MCP-1_1 | −2.807517 | 0.60158 | 0.102881 | 2 |
| IL-5_1 | −2.533339 | 0.11191 | 0.102881 | 2 |
| IGF-1_1 | −2.429866 | 0.60042 | 0.102881 | 2 |
| LIGHT_1 | −1.739557 | 0.68069 | 0.102881 | 2 |
| GCP-2_1 | −1.69179 | 0.3493 | 0.102881 | 2 |
| Fractalkine | −1.687498 | 0.59612 | 0.102881 | 2 |
| IL-1ra_1 | −1.589684 | 0.78477 | 0.200803 | 2 |
| Fit-3 Ligan [symbol] | −1.113565 | 0.67551 | 1.190476 | 2 |
| IFN-g_1 | −3.560171 | 0.58458 | 0.102881 | 3 |
| MIP-1d_1 | −3.163485 | 0.71538 | 0.102881 | 3 |
| IL-6_1 | −2.794102 | 0.48921 | 0.102881 | 3 |
| CK b8-1_1 | −2.589929 | 0.68946 | 0.102881 | 3 |
| BMP-6_1 | −2.434357 | 0.72473 | 0.102881 | 3 |
| Eotaxin-2_{—} | −2.356828 | 0.7222 | 0.102881 | 3 |
| CNTF_1 | −2.309291 | 0.75875 | 0.102881 | 3 |
| MIP-3a_1 | −2.029226 | 0.70276 | 0.102881 | 3 |
| MIG_1 | −1.894224 | 0.72898 | 0.102881 | 3 |
| TGF-b_1 | −1.782306 | 0.70401 | 0.102881 | 3 |
| BMP-4_1 | −0.922924 | 0.92324 | 1.697531 | 3 |
| IGFBP-4_1 | −2.630045 | 0.5017 | 0.102881 | 4 |
| IL-7_1 | −0.692426 | 0.40835 | 2.19697 | 4 |
| PDGF-BB_{—} | −1.153073 | 0.79665 | 1.121795 | 5 |
| GM-CSF_1 | −3.318119 | 0.16273 | 0.102881 | 7 |
| SCF_1 | −2.478851 | 0.6653 | 0.102881 | 7 |
| IL-10_1 | −1.864524 | 0.3965 | 0.102881 | 7 |
| IL-13_1 | −1.538539 | NA | 0.200803 | 7 |
| GRO-a_1 | −1.338516 | 0.47248 | 0.531915 | 7 |
| FGF-7_1 | −1.147464 | 0.55216 | 1.121795 | 7 |
| BDNF_1 | −0.877883 | 0.9095 | 1.75841 | 7 |

[symbol]

indicates data missing or illegible when filed

TABLE 17A

| Name | Score(d) | Fold Change | q-value (%) |
|---|---|---|---|
| NAP-2_1 | 3.015803 | 2.3311 | 0.416666667 |
| ANG_1 | 2.7793114 | 2.0092 | 0.416666667 |
| PARC_1 | 2.7552638 | 2.63872 | 0.416666667 |
| ICAM-1_1 | 2.5183244 | 2.54462 | 0.416666667 |
| IL-6 R_1 | 2.1634336 | 2.07358 | 0.416666667 |
| BTC_1 | 2.1006544 | 2.19149 | 0.416666667 |
| Acrp30_1 | 2.0335818 | 3.65294 | 0.416666667 |
| MSP-a_1 | 2.0025957 | 2.39185 | 0.416666667 |
| sTNF RII_1 | 1.9686306 | 2.15344 | 0.416666667 |
| TIMP-2_1 | 1.871601 | 1.99706 | 0.416666667 |
| TRAIL R3_1 | 1.833582 | 2.20251 | 0.416666667 |
| ANG-2_1 | 1.7806394 | 2.07536 | 0.416666667 |
| IL-8_1 | 1.7332094 | 2.02022 | 0.416666667 |
| AXL_1 | 1.6501027 | 1.883 | 0.416666667 |
| MIF_1 | 1.6434624 | 2.22659 | 0.416666667 |
| TIMP-1_1 | 1.5836883 | 1.7417 | 0.416666667 |
| MIP-1b_1 | 1.5753303 | 2.36633 | 0.416666667 |
| IGFBP-6_1 | 1.4684802 | 1.92629 | 0.416666667 |
| spg130_1 | 1.391691 | 2.1923 | 0.416666667 |
| CTACK_1 | 1.3483897 | 1.72505 | 0.416666667 |
| IGFBP3_1 | 1.3384955 | 1.84934 | 0.416666667 |
| uPAR_1 | 1.3349356 | 2.42069 | 0.416666667 |
| MIP-1a_1 | 1.3186579 | 1.931 | 0.416666667 |
| TRAIL R4_1 | 1.3116694 | 1.98605 | 0.416666667 |
| IL-12 p40_1 | 1.2911168 | 1.63912 | 0.416666667 |
| AR_1 | 1.2206417 | 2.15904 | 0.416666667 |
| TPO_1 | 1.2044047 | 1.86455 | 0.416666667 |
| NT-4_1 | 1.1793811 | 2.41703 | 0.416666667 |
| FAS_1 | 1.169934 | 1.59942 | 0.416666667 |
| bFGF | 1.1482616 | 1.58016 | 0.416666667 |
| VEGF-B_1 | 1.1358842 | 1.89024 | 0.416666667 |
| VEGF-D_1 | 1.0974084 | 3.07633 | 0.416666667 |
| OSM_1 | 1.0240581 | 1.8449 | 0.416666667 |
| OST_1 | 0.9845184 | 1.82276 | 0.416666667 |
| IL-11_1 | 0.9675503 | 2.26315 | 0.416666667 |
| sTNF RI_1 | 0.9627974 | 1.96913 | 0.416666667 |
| RANTES_1 | 0.9456799 | 1.34024 | 0.416666667 |
| I-TAC_1 | 0.9164841 | 2.27116 | 0.416666667 |
| Eotaxin_1 | 0.8908395 | 1.46174 | 1.215277778 |
| TECK_1 | 0.8828589 | 1.77056 | 1.215277778 |
| PIGF_1 | 0.8283546 | 2.16487 | 1.215277778 |
| b-NGF_1 | 0.8160618 | 1.60576 | 1.215277778 |
| EGF-R_1 | 0.7960517 | 1.41315 | 1.215277778 |
| Lymphotactin_1 | 0.7585063 | 1.55228 | 1.215277778 |
| MIP-3b_1 | 0.7025106 | 1.81688 | 2.5 |
| HCC-4_1 | 0.6557043 | 1.70769 | 2.5 |
| ICAM-3_1 | 0.6370118 | 1.72939 | 3.012048193 |
| IGFBP-2_1 | 0.6208166 | 1.2029 | 3.012048193 |
| DTK_1 | 0.5615526 | 1.50254 | 3.63372093 |
| IL-1 RI_1 | 0.5347156 | 1.73834 | 3.93258427 |
| IGF-1 SR | 0.5135245 | 1.5253 | 3.93258427 |
| AgRP(ART)_1 | 0.5124192 | 1.82258 | 3.93258427 |
| GRO_1 | 0.4666771 | 1.31521 | 5.163043478 |
| GITR-Light_1 | 0.4504103 | 1.38962 | 5.859375 |
| IGFBP-1_1 | 0.4352987 | 1.20224 | 5.859375 |
| HGF_1 | 0.4038156 | 1.33883 | 6.18556701 |
| IL-1R4/ST2_1 | 0.2875716 | 1.22954 | 9.926470588 |
| IL-2 Ra_1 | 0.25742 | 1.2669 | 10.71428571 |
| ENA-78_1 | 0.2468783 | 1.29573 | 10.71428571 |
| FGF-9_1 | 0.2420414 | 1.23628 | 10.71428571 |

TABLE 17B

| Gene Name | Score(d) | Fold Change | q-value (%) |
|---|---|---|---|
| MCP-2_1 | −2.304292 | 0.22807 | 0.416666667 |
| IL-1ra_1 | −2.207305 | 0.55921 | 0.416666667 |
| M-CSF_1 | −2.0793884 | 0.38905 | 0.416666667 |
| MCP-1_1 | −2.0252914 | 0.4534 | 0.416666667 |
| IL-3_1 | −1.9497211 | 0.33125 | 0.416666667 |
| MCP-3_1 | −1.8900971 | 0.29936 | 0.416666667 |
| MDC_1 | −1.7837426 | 0.44485 | 0.416666667 |
| MCP-4_1 | −1.7161914 | 0.24506 | 0.416666667 |
| IL-1b_1 | −1.7090727 | 0.25335 | 0.416666667 |
| BMP-6_1 | −1.601608 | 0.60317 | 0.416666667 |
| IL-4_1 | −1.5566673 | 0.46009 | 0.416666667 |
| IL-1a_1 | −1.5383795 | 0.31159 | 0.416666667 |
| BLC_1 | −1.5068668 | 0.48287 | 0.416666667 |
| CNTF_1 | −1.4946707 | 0.6341 | 0.416666667 |
| CK b8-1_1 | −1.4772423 | 0.56519 | 0.416666667 |
| IL-2_1 | −1.4647542 | 0.30616 | 0.416666667 |
| IFN-g_1 | −1.3743866 | 0.55449 | 0.416666667 |
| IL-15_1 | −1.2793787 | 0.22092 | 0.416666667 |
| Eotaxin-2_1 | −1.2356313 | 0.64369 | 0.416666667 |
| MIP-3a_1 | −1.2249652 | 0.56046 | 0.416666667 |
| MIG_1 | −1.169439 | 0.59839 | 0.416666667 |
| SCF_1 | −1.0907746 | 0.62327 | 0.416666667 |
| IL-6_1 | −1.0435505 | 0.43341 | 1.215277778 |
| PDGF-BB_1 | −1.0262008 | 0.68948 | 1.215277778 |
| IL-16_1 | −0.9969314 | 0.23613 | 1.215277778 |
| Eotaxin-3_1 | −0.9674019 | 0.52064 | 1.215277778 |
| I-309_1 | −0.941786 | 0.54744 | 1.215277778 |
| TGF-b_1 | −0.9411308 | 0.59424 | 1.215277778 |
| TNF-a_1 | −0.9018304 | 0.58157 | 1.623376623 |
| FGF-6_1 | −0.897254 | 0.63694 | 1.623376623 |
| GDNF_1 | −0.8697946 | 0.60042 | 1.623376623 |
| MIP-1d_1 | −0.8577233 | 0.77094 | 1.623376623 |
| LIGHT_1 | −0.8539608 | 0.606 | 1.623376623 |
| SDF-1_1 | −0.807095 | 0.60929 | 2.5 |
| IGF-1_1 | −0.7466459 | 0.61547 | 3.012048193 |
| Fractalkine_1 | −0.7310159 | 0.51894 | 3.63372093 |
| BDNF_1 | −0.7223848 | 0.82491 | 3.63372093 |
| IL-5_1 | −0.6300046 | 0.12006 | 4.532967033 |
| TGF-b3_1 | −0.6228815 | 0.8205 | 4.532967033 |
| BMP-4_1 | −0.5789929 | 0.87844 | 5.319148936 |
| Fit-3 Ligand_1 | −0.5692741 | 0.55604 | 5.319148936 |
| GM-CSF_1 | −0.5288316 | 0.25808 | 6.565656566 |
| IGFBP-4_1 | −0.5086457 | 0.69375 | 6.565656566 |
| GCP-2_1 | −0.4309765 | 0.37597 | 7.5 |
| TARC_1 | −0.4088338 | 0.59042 | 7.673267327 |

TABLE 18A

| Name | Score(d) | Fold Change | q-value (%) |
|---|---|---|---|
| TRAIL R4_1 | 2.264750916 | NA | 0.904761905 |
| Eotaxin_1 | 1.93445339 | 4.70062 | 0.904761905 |
| IL-12 p40_1 | 1.880163267 | 3.86536 | 0.904761905 |
| BTC_1 | 1.792904474 | 2.4468 | 0.904761905 |
| IL-8_1 | 1.623999996 | 2.67095 | 0.904761905 |
| MIF_1 | 1.578135137 | 2.79532 | 0.904761905 |
| MSP-a_1 | 1.541907487 | 2.11334 | 0.904761905 |
| uPAR_1 | 1.392662122 | 4.38083 | 0.904761905 |
| OST_1 | 1.357147945 | 6.61147 | 0.904761905 |
| MIP-1a_1 | 1.131822882 | 2.18476 | 0.904761905 |
| TPO_1 | 1.127049496 | 2.28982 | 0.904761905 |
| TRAIL R3_1 | 1.092119228 | 1.61261 | 0.904761905 |
| TGF-b3_1 | 1.043970414 | 1.99067 | 0.904761905 |
| sTNF RII_1 | 1.033890515 | 1.55451 | 0.904761905 |
| GCSF_1 | 1.024951701 | 3.10372 | 0.904761905 |
| sTNF RI_1 | 1.014653009 | 2.78772 | 0.904761905 |
| IL-11_1 | 1.00391809 | 5.07851 | 0.904761905 |
| MIP-1b_1 | 0.9966162 | 1.83838 | 0.904761905 |
| VEGF-B_1 | 0.94194004 | 2.00884 | 0.904761905 |
| Lymphotactin_1 | 0.935601365 | 2.41527 | 0.904761905 |
| NT-4_1 | 0.923994255 | 2.57292 | 0.904761905 |
| VEGF-D_1 | 0.898048249 | 3.15089 | 0.904761905 |
| Acrp30_1 | 0.885692332 | 1.51332 | 0.904761905 |
| HGF_1 | 0.84992308 | 4.96263 | 0.904761905 |
| IGFBP3_1 | 0.792485456 | 1.54086 | 0.904761905 |
| IGFBP-1_1 | 0.784580171 | 1.62237 | 0.904761905 |
| OSM_1 | 0.748360453 | 1.76423 | 0.904761905 |
| IL-1 RI_1 | 0.744755448 | 6.0184 | 0.904761905 |
| PIGF_1 | 0.723608877 | 2.81402 | 1.544715447 |
| IGF-1 SR | 0.708495305 | 3.05733 | 1.544715447 |
| RANTES | 0.701613901 | 1.26004 | 1.544715447 |
| ICAM-1_1 | 0.644564318 | 1.24206 | 2.753623188 |
| CCL-28_1 | 0.587722077 | 5.65125 | 3.298611111 |
| IL-1ra_1 | 0.555953031 | 1.3324 | 5.61827957 |
| IL-2 Ra_1 | 0.551415381 | 2.80849 | 5.61827957 |
| PARC_1 | 0.518735944 | 1.15104 | 5.61827957 |
| FAS_1 | 0.507008801 | 1.28116 | 5.61827957 |
| IL-12 p70_1 | 0.487911594 | 3.29805 | 5.61827957 |
| NAP-2_1 | 0.484247072 | 1.11825 | 5.61827957 |
| GRO_1 | 0.461543045 | 1.44588 | 5.61827957 |
| NT-3_1 | 0.410047836 | 1.32477 | 7.6 |
| IGFBP-6_1 | 0.408420436 | 1.21894 | 7.6 |
| TIMP-1_1 | 0.400113082 | 1.14706 | 7.6 |
| IL-17_1 | 0.392498707 | 2.73288 | 7.6 |
| IGFBP-2_1 | 0.38618776 | 1.16272 | 7.6 |
| CTACK_1 | 0.380915566 | 1.19299 | 7.6 |
| I-TAC_1 | 0.370637104 | 1.4308 | 7.6 |
| ICAM-3_1 | 0.338506181 | 1.47039 | 8.417721519 |
| ANG-2_1 | 0.335369663 | 1.14941 | 8.417721519 |
| FGF-4_1 | 0.311494132 | 1.91614 | 9.104166667 |
| MIP-3b_1 | 0.293878941 | 1.34124 | 9.728915663 |
| FGF-9_1 | 0.293742777 | 1.46816 | 9.728915663 |
| HCC-4_1 | 0.263286334 | 1.29481 | 11.61111111 |
| IL-1R4/ST2_1 | 0.252559948 | 1.32988 | 11.61111111 |
| ANG_1 | 0.248721281 | 1.05528 | 11.61111111 |
| GITR_1 | 0.247865761 | 1.33642 | 11.61111111 |
| DTK_1 | 0.241137412 | 1.25033 | 11.61111111 |
| IL-6 R_1 | 0.225218631 | 1.072 | 12.04710145 |
| EGF-R_1 | 0.193331739 | 1.1082 | 13.81205674 |

TABLE 18B

| Name | Score(d) | Fold Change | q-value (%) |
|---|---|---|---|
| IL-1a_1 | −1.425685763 | 0.28059 | 0.904761905 |
| MCP-2_1 | −1.212675578 | 0.30691 | 0.904761905 |
| IGFBP-4_1 | −1.20895142 | 0.39001 | 0.904761905 |
| spg130_1 | −1.199429488 | 0.61096 | 0.904761905 |
| SDF-1_1 | −1.153623548 | 0.44789 | 0.904761905 |
| M-CSF_1 | −1.111197881 | 0.48295 | 0.904761905 |
| MIP-1d_1 | −1.070072417 | 0.65762 | 0.904761905 |
| IL-10_1 | −1.009846401 | 0.25518 | 1.544715447 |
| GM-CSF_1 | −0.958718459 | 0.11603 | 1.544715447 |
| TNF-a_1 | −0.934948264 | 0.49119 | 1.544715447 |
| MDC_1 | −0.869780931 | 0.55252 | 2.753623188 |
| FGF-6_1 | −0.846319232 | 0.58971 | 2.753623188 |
| TNF-b_1 | −0.842647499 | 0.72752 | 2.753623188 |
| IFN-g_1 | −0.831081042 | 0.60989 | 2.753623188 |
| GDNF_1 | −0.790743331 | 0.55062 | 3.298611111 |
| Eotaxin-3_1 | −0.7492123 | 0.51758 | 5.61827957 |
| MCP-3_1 | −0.643949943 | 0.49699 | 5.61827957 |
| BLC_1 | −0.635584231 | 0.621 | 5.61827957 |
| IGF-1_1 | −0.626811933 | 0.59071 | 5.61827957 |
| TARC_1 | −0.621812924 | 0.407 | 5.61827957 |
| IL-13_1 | −0.606932031 | NA | 5.61827957 |
| AXL_1 | −0.602711809 | 0.80618 | 5.61827957 |
| GRO-a_1 | −0.535561363 | 0.42506 | 7.6 |
| IL-1b_1 | −0.527429339 | 0.48739 | 7.6 |
| SCF_1 | −0.523648284 | 0.72671 | 7.6 |
| IL-5_1 | −0.523276967 | 0.10826 | 7.6 |
| IL-16_1 | −0.519147838 | 0.30682 | 7.6 |
| I-309_1 | −0.512084847 | 0.61731 | 7.6 |
| TECK_1 | −0.483535641 | 0.76083 | 8.417721519 |
| AgRP(ART)_1 | −0.472803161 | 0.6455 | 8.417721519 |
| IL-6_1 | −0.44191818 | 0.57236 | 9.728915663 |
| IL-15_1 | −0.41494314 | 0.38371 | 11.61111111 |
| GCP-2_1 | −0.401329611 | 0.31787 | 11.61111111 |
| MCP-4_1 | −0.392420281 | 0.52574 | 12.04710145 |
| Eotaxin-2_1 | −0.354478448 | 0.82923 | 13.81205674 |
| IL-2_1 | −0.343716173 | 0.58707 | 13.85416667 |
| IL-4_1 | −0.334158663 | 0.74801 | 13.85416667 |
| FGF-7_1 | −0.31567674 | 0.48289 | 14.21768707 |
| LIGHT_1 | −0.307045767 | 0.77313 | 14.21768707 |
| IL-3_1 | −0.288230929 | 0.71595 | 14.39393939 |
| Fractalkine_1 | −0.255510085 | 0.69456 | 16.77392739 |
| IL-7_1 | −0.212551274 | 0.37996 | 16.77392739 |
| CK b8-1_1 | −0.171953761 | 0.89232 | 18.0952381 |
| BMP-6_1 | −0.165427865 | 0.918 | 18.0952381 |
| LEPTIN(OB)_1 | −0.162080603 | 0.88435 | 18.0952381 |
| MCP-1_1 | −0.157017681 | 0.8931 | 18.0952381 |

TABLE 19A

| Name | Score(d) | Fold Change | q-value (%) |
|---|---|---|---|
| NAP-2_1 | 4.267334 | 2.25145 | 0.694444 |
| ANG_1 | 4.061566 | 1.97693 | 0.694444 |
| AXL_1 | 3.946682 | 2.03097 | 0.694444 |
| PARC_1 | 3.740647 | 2.53113 | 0.694444 |
| ICAM-1_1 | 3.510347 | 2.38945 | 0.694444 |
| IL-6 R_1 | 3.397778 | 2.02276 | 0.694444 |
| spg130_1 | 3.297869 | 2.61126 | 0.694444 |
| ANG-2_1 | 3.253421 | 1.98738 | 0.694444 |
| AR_1 | 2.780729 | 2.195 | 0.694444 |
| IGFBP-6_1 | 2.766085 | 1.81674 | 0.694444 |
| TIMP-2_1 | 2.746738 | 1.96642 | 0.694444 |
| sTNF RII_ [symbol] | 2.70119 | 1.9052 | 0.694444 |
| BTC_1 | 2.354153 | 1.77895 | 0.694444 |
| Acrp30_1 | 2.292376 | 3.26933 | 0.694444 |
| CTACK_1 | 2.286645 | 1.63476 | 0.694444 |
| bFGF | 2.254793 | 1.59862 | 0.694444 |
| TIMP-1_1 | 2.203826 | 1.67415 | 0.694444 |
| TRAIL R3_ [symbol] | 2.143125 | 1.93754 | 0.694444 |
| MSP-a_1 | 2.110976 | 1.99091 | 0.694444 |
| MIP-1b_1 | 2.086051 | 2.01983 | 0.694444 |
| FAS_1 | 2.059914 | 1.48374 | 0.694444 |
| IGFBP3_1 | 1.955992 | 1.63927 | 1.092896 |
| TECK_1 | 1.799893 | 1.93772 | 1.092896 |
| IL-8_1 | 1.798862 | 1.61555 | 1.092896 |
| b-NGF_1 | 1.772438 | 1.60984 | 1.092896 |
| MIF_1 | 1.695812 | 1.77156 | 1.092896 |
| MIP-1a_1 | 1.679684 | 1.59738 | 1.092896 |
| NT-4_1 | 1.61208 | 1.94614 | 1.092896 |
| EGF-R_1 | 1.607028 | 1.36793 | 1.092896 |
| I-TAC_1 | 1.557412 | 2.05114 | 3.196347 |
| OSM_1 | 1.48 | 1.59379 | 3.196347 |
| TPO_1 | 1.401631 | 1.53133 | 3.196347 |
| VEGF-B_1 | 1.386749 | 1.58684 | 3.196347 |
| VEGF-D_1 | 1.343569 | 2.40993 | 3.196347 |
| uPAR_1 | 1.32707 | 1.82461 | 3.196347 |
| MIP-3b_1 | 1.264924 | 1.66183 | 3.196347 |
| AgRP(ART | 1.184203 | 2.12294 | 4.819277 |
| PIGF_1 | 1.121384 | 1.71402 | 4.819277 |
| HCC-4_1 | 1.115816 | 1.57811 | 4.819277 |
| IL-11_1 | 1.111969 | 1.67652 | 4.819277 |
| DTK_1 | 1.089757 | 1.40526 | 4.819277 |
| sTNF RI_1 | 1.083266 | 1.57406 | 4.819277 |
| TNF-b_1 | 1.064988 | 1.18835 | 4.819277 |
| ICAM-3_1 | 1.047944 | 1.5309 | 4.819277 |
| RANTES_ [symbol] | 1.039346 | 1.25415 | 4.819277 |

[symbol]

indicates data missing or illegible when filed

TABLE 19B

| Name | Score(d) | Fold Change | q-value (%) |
|---|---|---|---|
| IL-1ra_1 | −5.041602 | 0.51632 | 0.694444 |
| IL-3_1 | −4.65699 | 0.37506 | 0.694444 |
| MCP-1_1 | −4.613776 | 0.47067 | 0.694444 |
| MCP-4_1 | −4.073299 | 0.31815 | 0.694444 |
| MCP-3_1 | −3.883939 | 0.40145 | 0.694444 |
| MCP-2_1 | −3.794381 | 0.40233 | 0.694444 |
| CK b8-1_1 | −3.694038 | 0.58898 | 0.694444 |
| CNTF_1 | −3.611565 | 0.64605 | 0.694444 |
| IL-1b_1 | −3.539065 | 0.34043 | 0.694444 |
| BMP-6_1 | −3.532174 | 0.62281 | 0.694444 |
| IL-2_1 | −3.525665 | 0.37899 | 0.694444 |
| IL-4_1 | −3.512443 | 0.51003 | 0.694444 |
| M-CSF_1 | −3.435204 | 0.5251 | 0.694444 |
| MIP-3a_1 | −3.223006 | 0.57152 | 0.694444 |
| MDC_1 | −3.136714 | 0.56385 | 0.694444 |
| BLC_1 | −2.977992 | 0.57752 | 0.694444 |
| MIG_1 | −2.84823 | 0.61226 | 0.694444 |
| IL-15_1 | −2.83153 | 0.33554 | 0.694444 |
| Eotaxin-2_ [symbol] | −2.466855 | 0.68813 | 0.694444 |
| IFN-g_1 | −2.339649 | 0.66411 | 0.694444 |
| TGF-b3_1 | −2.302077 | 0.68801 | 0.694444 |
| TGF-b_1 | −2.237739 | 0.6243 | 0.694444 |
| IL-6_1 | −2.232468 | 0.54172 | 0.694444 |
| IL-16_1 | −2.116464 | 0.41262 | 0.694444 |
| IL-1a_1 | −1.926189 | 0.57411 | 0.694444 |
| I-309_1 | −1.895322 | 0.65572 | 0.694444 |
| SCF_1 | −1.888043 | 0.70339 | 0.694444 |
| LIGHT_1 | −1.703026 | 0.66186 | 1.092896 |
| PDGF-BB_ [symbol] | −1.661166 | 0.70275 | 1.092896 |
| BDNF_1 | −1.610622 | 0.82141 | 1.092896 |
| Fractalkine | −1.601759 | 0.59002 | 1.092896 |
| Eotaxin-3_ [symbol] | −1.528746 | 0.69067 | 1.092896 |
| Fit-3 Ligan [symbol] | −1.421242 | 0.58491 | 3.196347 |
| GCSF_1 | −1.236217 | 0.70092 | 3.196347 |
| GDNF_1 | −1.233345 | 0.75441 | 3.196347 |
| BMP-4_1 | −1.194332 | 0.88628 | 3.196347 |
| FGF-6_1 | −1.183592 | 0.78548 | 3.196347 |
| IGF-1_1 | −1.132697 | 0.75456 | 4.819277 |
| IL-5_1 | −1.102411 | 0.44825 | 5.098039 |
| TNF-a_1 | −1.087972 | 0.779 | 5.098039 |

[symbol]

indicates data missing or illegible when filed

TABLE 20A

| Protein Name | Score(d) | Fold Change | p-value (%) |
|---|---|---|---|
| BTC_1 | 5.280599 | 2.30404 | 0.106838 |
| IL-8_1 | 4.611751 | 2.30142 | 0.106838 |
| IL-12 p40_1 | 4.397923 | 2.30237 | 0.106838 |
| TRAIL R4_1 | 4.18957 | 4.38847 | 0.106838 |
| MIF_1 | 3.78626 | 2.46763 | 0.106838 |
| MIP-1a_1 | 3.671968 | 2.04509 | 0.106838 |
| sTNF RII_1 | 3.57664 | 1.81136 | 0.106838 |
| MSP-a_1 | 3.532718 | 2.23649 | 0.106838 |
| OST_1 | 3.519536 | 2.85493 | 0.106838 |
| IL-11_1 | 3.428231 | 3.16541 | 0.106838 |
| uPAR_1 | 3.42578 | 3.10753 | 0.106838 |
| TPO_1 | 3.260328 | 2.04533 | 0.106838 |
| NT-4_1 | 3.182778 | 2.48474 | 0.106838 |
| MIP-1b_1 | 3.119065 | 2.07252 | 0.106838 |
| NAP-2_1 | 2.970365 | 1.51262 | 0.106838 |
| ICAM-1_1 | 2.949073 | 1.6633 | 0.106838 |
| IGFBP3_1 | 2.868921 | 1.68668 | 0.106838 |
| TRAIL R3_1 | 2.808197 | 1.85516 | 0.106838 |
| Eotaxin_1 | 2.747874 | 2.23776 | 0.106838 |
| VEGF-B_1 | 2.73066 | 1.94657 | 0.106838 |
| PARC_1 | 2.703205 | 1.59801 | 0.106838 |
| Lymphotactin_1 | 2.655294 | 1.92588 | 0.106838 |
| sTNF RI_1 | 2.628389 | 2.27051 | 0.106838 |
| PIGF_1 | 2.59266 | 2.46572 | 0.106838 |
| ANG-2_1 | 2.573094 | 1.48217 | 0.106838 |
| IGFBP-6_1 | 2.559164 | 1.49096 | 0.106838 |
| OSM_1 | 2.548107 | 1.79103 | 0.106838 |
| ANG_1 | 2.527071 | 1.38167 | 0.106838 |
| FAS_1 | 2.522175 | 1.42939 | 0.106838 |

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

TABLE 20B

| Name | Score(d) | Fold Change | p-value (%) |
|---|---|---|---|
| MCP-2_1 | −5.82991 | 0.25732 | 0.106838 |
| M-CSF_1 | −5.0083 | 0.42889 | 0.106838 |
| IL-1a_1 | −4.92065 | 0.29231 | 0.106838 |
| MDC_1 | −4.36259 | 0.48973 | 0.106838 |
| MCP-3_1 | −4.03467 | 0.36994 | 0.106838 |
| SDF-1_1 | −3.71753 | 0.51302 | 0.106838 |
| BLC_1 | −3.62482 | 0.54297 | 0.106838 |
| IFN-g_1 | −3.56017 | 0.58458 | 0.106838 |
| TNF-a_1 | −3.50252 | 0.52906 | 0.106838 |
| MCP-4_1 | −3.39139 | 0.33264 | 0.106838 |
| Eotaxin-3_1 | −3.37887 | 0.50745 | 0.106838 |
| GM-CSF_1 | −3.31812 | 0.16273 | 0.106838 |
| IL-3_1 | −3.29267 | 0.45124 | 0.106838 |
| IL-1b_1 | −3.2351 | 0.33216 | 0.106838 |
| MIP-1d_1 | −3.16349 | 0.71538 | 0.106838 |
| IL-16_1 | −3.11242 | 0.26418 | 0.106838 |
| IL-2_1 | −3.09127 | 0.39923 | 0.106838 |
| FGF-6_1 | −2.99526 | 0.60629 | 0.106838 |
| IL-15_1 | −2.99089 | 0.2798 | 0.106838 |
| IL-4_1 | −2.90998 | 0.56937 | 0.106838 |
| GDNF_1 | −2.89861 | 0.57687 | 0.106838 |
| I-309_1 | −2.81343 | 0.58059 | 0.106838 |
| MCP-1_1 | −2.80752 | 0.60158 | 0.106838 |
| IL-6_1 | −2.7941 | 0.48921 | 0.106838 |

We claim:

1. A method of aiding diagnosis of Alzheimer's disease (“AD”), comprising comparing a measured level of at least sixteen AD diagnosis biomarkers in a biological fluid sample from an individual seeking a diagnosis for AD to a reference level for each biomarker, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha).

2. The method of claim 1, wherein said biological fluid sample is a peripheral biological fluid sample.

3. The method of claim 2, wherein said peripheral biological fluid sample is serum or plasma.

4. The method of claim 1, further comprising obtaining a measured level of said AD biomarker in said biological fluid sample.

5. The method of claim 1, wherein the individual is a human.

6. The method of claim 1, wherein the measured levels are normalized.

7. The method of claim 1, wherein the reference levels are obtained from measured values of the at least sixteen biomarkers from samples in the blood of human individuals without AD.

8. The method of claim 1, wherein the reference levels are obtained from measured values of the at least sixteen biomarkers from samples in the blood of human individuals with AD.

9. The method of claim 7, wherein the reference levels are normalized.

10. The method of claim 1, wherein the method comprises comparing the measured level of the at least sixteen AD diagnosis biomarkers to two reference levels for each biomarker.

11. The method of claim 10, wherein the two reference levels for each biomarker comprise:
(a) a reference level obtained from measured values of the at least sixteen biomarkers from samples in the blood of human individuals without AD; and
(b) a reference level obtained from measured values of the at least sixteen biomarkers from samples in the blood of human individuals with AD.

12. The method of claim 7, wherein the group of individuals without AD is a control population selected from an age-matched population, a degenerative control population, a non-AD neurodegenerative control population, a healthy age-matched control population, or a mixed population thereof.

13. The method of claim 1, wherein comparing comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic, Logistic, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, and Ordinal Classifier.

14. The method of claim 1, wherein comparing comprises a method comprising predication analysis of microarray (PAM).

15. The method of claim 1, further comprising:
formulating a decision tree; and
using the decision tree for classification of the sample from the individual seeking AD diagnosis, wherein the classification aids the diagnosis of AD.

16. The method of claim 15, wherein using the decision tree for classification of the sample is implemented by a computer.

17. The method of claim 1, whereby the diagnosis of AD is aided by determining a difference between the measured levels of the at least sixteen AD diagnosis biomarkers to the reference levels of the at least sixteen biomarkers wherein the difference meets or exceeds a statistically significant difference between normalized measured values of the at least sixteen AD diagnosis biomarkers in the blood samples from individuals without AD and individuals with AD, wherein the statistically significant difference indicates a diagnosis of AD,
wherein the measured levels are normalized, and
wherein the references levels are normalized.

18. The method of claim 17, further comprising:
formulating a decision tree comprising statistically significant differences in normalized measured values of AD diagnosis biomarkers wherein the statistically significant differences are determined from normalized measured values of the plurality of AD diagnosis biomarkers in the blood samples in the group of individuals with AD and the group of individuals without AD; and
using the decision tree for classification of the blood sample from the individual seeking AD diagnosis, wherein the classification aids the diagnosis of AD.

19. The method of claim 1, wherein the method is useful for early detection of conversion of MCI to AD.

20. The method of claim 1, wherein the at least sixteen AD diagnosis biomarkers further comprise: TRAIL R4, and IGFBP-6.

21. The method of claim 1, further comprising the step of obtaining a value for each comparison of the measured level to the reference level.

22. A computer readable format comprising the values obtained by the method of claim 21.

23. A method for monitoring progression of Alzheimer's disease (AD) in an AD patient, comprising: comparing a measured level of at least sixteen AD diagnosis biomarkers in a biological fluid sample from an individual seeking a diagnosis for AD to a reference level for each biomarker, wherein the at least sixteen AD diagnosis biomarkers comprise: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha).

24. A kit comprising:
at least one reagent specific for each of at least sixteen AD diagnosis biomarkers, said at least sixteen AD diagnosis biomarkers comprising: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha; and
instructions for carrying out the method of claim 1.

25. A surface comprising attached thereto, at least one reagent specific for each of at least sixteen AD diagnosis biomarkers, said at least sixteen AD diagnosis biomarkers comprising: MCSF (Macrophage Colony Stimulating Factor), RANTES, GCSF (granulocyte-colony stimulating factor), PARC (pulmonary and activation-regulated chemokine), ANG-2 (angiotensin-2), IL-11 (interleukin-11), EGF (epidermal growth factor), MCP-3 (monocyte chemoattractant protein-3), IL-3 (interleukin-3), MIP-1delta (macrophage inflammatory protein-1 delta), ICAM-1 (intercellular adhesion molecule 1), PDGF-BB (platelet-derived growth factor BB), IL-8 (interleukin 8), GDNF (glial derived neurotrophic factor), IL-1a (interleukin-1alpha), and TNF-a (tumor necrosis factor alpha.
